SEQUENCE LISTING (81 sequences)

SEQ ID NO: 1 - protein, 5 aa, Artificial Sequence
  Synthetic construct
GYTMN

SEQ ID NO: 2 - protein, 17 aa, Artificial Sequence
  Synthetic construct
LINPYKGVSTYNQKFKD

SEQ ID NO: 3 - protein, 13 aa, Artificial Sequence
  Synthetic construct
SGYYGDSDWYFDV

SEQ ID NO: 4 - protein, 11 aa, Artificial Sequence
  Synthetic construct
RASQDIRNYLN

SEQ ID NO: 5 - protein, 7 aa, Artificial Sequence
  Synthetic construct
YTSRLES

SEQ ID NO: 6 - protein, 9 aa, Artificial Sequence
  Synthetic construct
QQGNTLPWT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 5 aa, Artificial Sequence
  Synthetic construct
SYAIS

SEQ ID NO: 10 - protein, 17 aa, Artificial Sequence
  Synthetic construct
GIIPIFGTANYAQKFQG

SEQ ID NO: 11 - protein, 11 aa, Artificial Sequence
  Synthetic construct
SIELWWGGFDY

SEQ ID NO: 12 - protein, 11 aa, Artificial Sequence
  Synthetic construct
RASQSISSWLA

SEQ ID NO: 13 - protein, 7 aa, Artificial Sequence
  Synthetic construct
DASSLES

SEQ ID NO: 14 - protein, 8 aa, Artificial Sequence
  Synthetic construct
QQYEDYTT

SEQ ID NO: 15 - protein, 120 aa, Artificial Sequence
  Synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSS

SEQ ID NO: 16 - protein, 106 aa, Artificial Sequence
  Synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIK

SEQ ID NO: 17 - protein, 213 aa, Artificial Sequence
  Synthetic construct
DIQMTQSPSTLSASVGDRVTITCRASQSISSWLAWYQQKPGKAPKLLIYDASSLESGVPSRFSGSGSGTEFTLTIGSLQPDDFATYYCQQYEDYTTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 448 aa, Artificial Sequence
  Synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 229 aa, Artificial Sequence
  Synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 671 aa, Artificial Sequence
  Synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSIELWWGGFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 22 - protein, 9 aa, Homo sapiens
VLDFAPPGA

SEQ ID NO: 23 - protein, 9 aa, Homo sapiens
RMFPNAPYL

SEQ ID NO: 24 - protein, 275 aa, Homo sapiens [sequence omitted]

SEQ ID NO: 25 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 26 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 27 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 28 - protein, 5 aa, Artificial Sequence
  Synthetic construct
TYAMN

SEQ ID NO: 29 - protein, 19 aa, Artificial Sequence
  Synthetic construct
RIRSKYNNYATYYADSVKG

SEQ ID NO: 30 - protein, 14 aa, Artificial Sequence
  Synthetic construct
HGNFGNSYVSWFAY

SEQ ID NO: 31 - protein, 14 aa, Artificial Sequence
  Synthetic construct
GSSTGAVTTSNYAN

SEQ ID NO: 32 - protein, 7 aa, Artificial Sequence
  Synthetic construct
GTNKRAP

SEQ ID NO: 33 - protein, 9 aa, Artificial Sequence
  Synthetic construct
ALWYSNLWV

SEQ ID NO: 34 - protein, 125 aa, Artificial Sequence
  Synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 35 - protein, 109 aa, Artificial Sequence
  Synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 36 - protein, 5 aa, Artificial Sequence
  Synthetic construct
EFGMN

SEQ ID NO: 37 - protein, 17 aa, Artificial Sequence
  Synthetic construct
WINTKTGEATYVEEFKG

SEQ ID NO: 38 - protein, 12 aa, Artificial Sequence
  Synthetic construct
WDFAYYVEAMDY

SEQ ID NO: 39 - protein, 11 aa, Artificial Sequence
  Synthetic construct
KASAAVGTYVA

SEQ ID NO: 40 - protein, 7 aa, Artificial Sequence
  Synthetic construct
SASYRKR

SEQ ID NO: 41 - protein, 10 aa, Artificial Sequence
  Synthetic construct
HQYYTYPLFT

SEQ ID NO: 42 - protein, 121 aa, Artificial Sequence
  Synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 43 - protein, 108 aa, Artificial Sequence
  Synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 44 - protein, 215 aa, Artificial Sequence
  Synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 451 aa, Artificial Sequence
  Synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 214 aa, Artificial Sequence
  Synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 47 - protein, 694 aa, Artificial Sequence
  Synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 5 aa, Artificial Sequence
  Synthetic construct
YSWIN

SEQ ID NO: 49 - protein, 17 aa, Artificial Sequence
  Synthetic construct
RIFPGDGDTDYNGKFKG

SEQ ID NO: 50 - protein, 10 aa, Artificial Sequence
  Synthetic construct
NVFDGYWLVY

SEQ ID NO: 51 - protein, 16 aa, Artificial Sequence
  Synthetic construct
RSSKSLLHSNGITYLY

SEQ ID NO: 52 - protein, 7 aa, Artificial Sequence
  Synthetic construct
QMSNLVS

SEQ ID NO: 53 - protein, 9 aa, Artificial Sequence
  Synthetic construct
AQNLELPYT

SEQ ID NO: 54 - protein, 119 aa, Artificial Sequence
  Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 112 aa, Artificial Sequence
  Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 56 - protein, 219 aa, Artificial Sequence
  Synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 447 aa, Artificial Sequence
  Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 58 - protein, 232 aa, Artificial Sequence
  Synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 672 aa, Artificial Sequence
  Synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 60 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 61 - protein, 5 aa, Artificial Sequence
  Synthetic construct
SYAMN

SEQ ID NO: 62 - protein, 14 aa, Artificial Sequence
  Synthetic construct
HTTFPSSYVSYYGY

SEQ ID NO: 63 - protein, 125 aa, Artificial Sequence
  Synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSS

SEQ ID NO: 64 - protein, 5 aa, Artificial Sequence
  Synthetic construct
SYAMN

SEQ ID NO: 65 - protein, 14 aa, Artificial Sequence
  Synthetic construct
ASNFPASYVSYFAY

SEQ ID NO: 66 - protein, 125 aa, Artificial Sequence
  Synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 67 - protein, 5 aa, Artificial Sequence
  Synthetic construct
DYIMH

SEQ ID NO: 68 - protein, 17 aa, Artificial Sequence
  Synthetic construct
YINPYNDGSKYTEKFQG

SEQ ID NO: 69 - protein, 12 aa, Artificial Sequence
  Synthetic construct
GTYYYGPQLFDY

SEQ ID NO: 70 - protein, 16 aa, Artificial Sequence
  Synthetic construct
KSSQSLETSTGTTYLN

SEQ ID NO: 71 - protein, 7 aa, Artificial Sequence
  Synthetic construct
RVSKRFS

SEQ ID NO: 72 - protein, 9 aa, Artificial Sequence
  Synthetic construct
LQLLEDPYT

SEQ ID NO: 73 - protein, 121 aa, Artificial Sequence
  Synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSS

SEQ ID NO: 74 - protein, 112 aa, Artificial Sequence
  Synthetic construct
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 75 - protein, 674 aa, Artificial Sequence
  Synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 76 - protein, 674 aa, Artificial Sequence
  Synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGGQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 77 - protein, 449 aa, Artificial Sequence
  Synthetic construct
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 78 - protein, 219 aa, Artificial Sequence
  Synthetic construct
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 232 aa, Artificial Sequence
  Synthetic construct
EVQLLESGGGLVQPGGSLRLSCAASGFQFSSYAMNWVRQAPGKGLEWVSRIRSKYNNYATYYADSVKGRFTISRDDSKNTLYLQMNSLRAEDTAVYYCVRHTTFPSSYVSYYGYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 232 aa, Artificial Sequence
  Synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 556 aa, Homo sapiens [sequence omitted]

(19) United States
(12) Patent Application Publication
(10) Pub. No.: US 2022/0088195 A1
(43) Pub. Date: Mar. 24, 2022

(54) Prevention or mitigation of T-cell bispecific antibody-related adverse effects

(71) Applicant: Hoffmann-La Roche Inc., Little Falls, NJ (US)

(72) Inventors: Christian Klein, Bonstetten (CH); Hélène Cécile Haegel, Illkirch-Graffenstaden (CH); Alberto Toso, Basel (CH); Gabrielle Leclercq, Zuerich (CH); Tina Zimmermann, Zurich (CH)

(73) Assignee: Hoffmann-La Roche Inc., Little Falls, NJ (US)

(21) Appl. No.: 17/448,729

(22) Filed: Sep. 24, 2021

(30) Foreign Application Priority Data
Sep. 24, 2020 (EP) 20198050.5
Oct. 13, 2020 (EP) 20201583.0
May 7, 2021 (EP) 21172627.8

(51) Int. Cl. A61K 39/395; A61K 31/506

(52) U.S. Cl. CPC A61K 39/39541; A61K 2039/545; A61K 31/506

(57) ABSTRACT

The present invention relates to the prevention or mitigation of adverse effects related to T cell bispecific antibodies, such as cytokine release syndrome. Specifically, the invention relates to the prevention or mitigation of such side effects using a tyrosine kinase inhibitor such as dasatinib.

### CROSS REFERENCE TO RELATED APPLICATIONS

This application is a continuation of European Patent Application No. 20198050.5, filed Sep. 24, 2020, European Patent Application No. 20201583.0, filed Sep. 24, 2020, and European Patent Application No. 21172627.8, filed May 7, 2021 which are incorporated herein by reference in their entirety.

### SEQUENCE LISTING

This application contains a Sequence Listing which has been submitted electronically in ASCII format and is hereby incorporated by reference in its entirety. Said ASCII copy, created on Sep. 15, 2021, is named P36412-US_Sequence_Listing.txt and is 106,954 bytes in size.

### FIELD OF THE INVENTION

The present invention relates to the prevention or mitigation of adverse effects related to T cell bispecific antibodies, such as cytokine release syndrome. Specifically, the invention relates to the prevention or mitigation of such side effects using a tyrosine kinase inhibitor such as dasatinib.

### BACKGROUND

T cell engagers or T cell bispecific antibodies (TCBs) are bispecific antibodies that with one binding moiety recognize a target cell antigen, e.g. a tumor antigen expressed on tumor cells, and with the other binding moiety the T cell receptor. TCBs hold great promise as cancer immunotherapeutics. Crosslinking of CD3 with target cell antigens triggers T cell activation, proliferation and cytokine release, leading to target cell killing (Bacac et al., Clin Cancer Res (2016) 22, 3286-97; Bacac et al., Oncoimmunology (2016) 5, e1203498). However, TCB treatment is sometimes associated with safety liabilities due to on-target on tumor, on target off tumor cytotoxic activity and cytokine release. One of the most common adverse effects reported for TCBs is Cytokine Release Syndrome (CRS). This complex clinical syndrome is characterized by fever, hypotension and respiratory deficiency and associated with the release of pro-inflammatory cytokines such as IL-6, TNF-α, IFN-γ, and IL-1β (see e.g. Shimabukuro-Vomhagen et al., J Immunother Cancer (2018) 6, 56). Off-tumor toxicity may occur if target antigens are expressed in healthy cells, which may potentially result in tissue damages and compromise the patient's safety. Approaches to mitigate these life-threatening toxicities, for example pharmacological blockade of T cell activation and proliferation induced by TCBs, are greatly needed. The tyrosine kinase inhibitor dasatinib was identified as a potent candidate that switches off functionality of CAR-T cells (Weber et al., Blood Advances (2019) 3, 711-7; Mestermann et al., Sci Transl Med (2019) 11, eaau5907). On the other hand, simultaneous administration of dasatinib with the T cell engager blinatumomab seemed not impair activity of the latter (Chiaretti et al., Blood (2019) 134 (Supplement 1), 740; Foe), et al., N Engl J Med (2020) 383, 1613-1623).

### DESCRIPTION OF THE INVENTION

The present inventors have found that a tyrosine kinase inhibitor, in particular dasatinib, may be used as a pharmacological on/off switch to mitigate off-tumor toxicities or CRS by T cell engaging therapies.

Using an in vitro model of target cell killing by human peripheral blood mononuclear cells, the inventors assessed the reversible effects of dasatinib combined with four exemplary TCBs (CEA-TCB, CD20-TCB and CD19-TCB, as examples of tumor surface targeting TCBs, and HLA-A2 WT-1-TCB, as an example of TCR-like-TCB) on T cell activation and proliferation, target cell killing and cytokine release. Killing assays using a dose response of dasatinib were conducted to define the threshold at which TCB-induced T cell activation was fully inhibited. Furthermore, the inventors propose that a dasatinib concentration below this threshold may be used to control TCB-induced cytokine release. These counteracting effects can be obtained at dasatinib concentrations which are clinically relevant doses and could be used either to induce a blockade of TCB-induced T cell activation in case CRS symptoms are not manageable with standard interventions or to reduce cytokine release as alternatives to TNF or IL-6R blockade. The data in the present Examples show that dasatinib can act as a reversible on/off switch for TCB-mediated T cell activation, which could be used to mitigate TCB-induced on- and off-tumor toxicities including CRS.

Accordingly, in a first aspect, the present invention provides a T cell bispecific antibody for use in the treatment of a disease in an individual, wherein said treatment comprises

(a) the administration of the T cell bispecific antibody to the individual, and
(b) the administration of a tyrosine kinase inhibitor (TKI) to the individual for the prevention or mitigation of an adverse effect related to the administration of the T cell bispecific antibody.

The invention further provides the use of a T cell bispecific antibody in the manufacture of a medicament for the treatment of a disease in an individual, wherein said treatment comprises

(a) the administration of the T cell bispecific antibody to the individual, and
(b) the administration of a tyrosine kinase inhibitor (TKI) to the individual for the prevention or mitigation of an adverse effect related to the administration of the T cell bispecific antibody.

The invention also provides a method for treatment of a disease in an individual, wherein said method comprises

(a) the administration of a T cell bispecific antibody to the individual, and
(b) the administration of a tyrosine kinase inhibitor (TKI) to the individual for the prevention or mitigation of an adverse effect related to the administration of the T cell bispecific antibody.

In another aspect, the invention provides a tyrosine kinase inhibitor (TKI) for use in the prevention or mitigation of an adverse effect related to the administration of a T cell bispecific antibody to an individual.

The invention further provides the use of a tyrosine kinase inhibitor (TKI) in the manufacture of a medicament for the prevention or mitigation of an adverse effect related to the administration of a T cell bispecific antibody to an individual.

The invention also provides a method for preventing or mitigating an adverse effect related to the administration of a T cell bispecific antibody to an individual, comprising the administration of a tyrosine kinase inhibitor (TKI) to the individual.

The T cell bispecific antibody for use, TKI for use, uses or methods described above and herein, may incorporate, singly or in combination, any of the features described in the following (unless the context dictates otherwise).

Terms are used herein as generally used in the art, unless otherwise defined herein.

In some aspects, the TKI is a Lck and/or Src kinase inhibitor. In more specific aspects, the TKI is dasatinib.

“Dasatinib” is a tyrosine kinase inhibitor (TKI). It is sold under the brand name Sprycel® (among others), for the treatment of certain cases of chronic myelogenous leukemia (CML) and acute lymphoblastic leukemia (ALL). Its CAS number, IUPAC name and chemical structure are shown below.

CAS number: 302962-49-8

IUPAC name: N-(2-chloro-6-methylphenyl)-2-[[6-[4-(2-hydroxyethyl)-1-piperazinyl]-2-methyl-4-pyrimidinyl]amino]-5-thiazole carboxamide monohydrate

Chemical Structure:

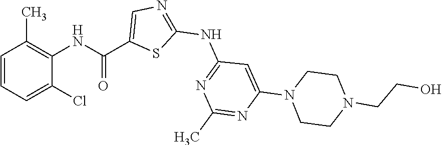 [SMILES: Cc1nc(Nc2ncc(C(=O)Nc3c(C)cccc3Cl)s2)cc(N2CCN(CCO)CC2)n1]

In some aspects, (administration of) the TKI causes inhibition of the activity of the T cell bispecific antibody.

“Activity” of a T cell bispecific antibody refers to responses in an individual's body caused by the T cell bispecific antibody. Such activity may include cellular response(s) of T cells, particularly CD4+ and/or CD8+ T cells, such as proliferation, differentiation, cytokine secretion, cytotoxic effector molecule release, cytotoxic activity, and expression of activation markers, and/or effects on target cells, particularly target cells (e.g. tumor cells) expressing the target cell antigen of the T cell bispecific antibody, such as lysis of target cells.

In some aspects, (administration of) the TKI causes inhibition of the activation of T cells (induced by the T cell bispecific antibody).

“Activation of T cells” or “T cell activation” as used herein refers to one or more cellular response of a T lymphocyte, particularly a CD4+ or CD8+ T cell, selected from: proliferation, differentiation, cytokine secretion, cytotoxic effector molecule release, cytotoxic activity, and expression of activation markers. Suitable assays to measure T cell activation are known in the art and described herein. In particular aspects, T cell activation is determined by measuring expression of CD25 and/or CD69 on the T cell, e.g. by flow cytometry.

In some aspects, (administration of) the TKI causes inhibition of the proliferation of T cells (induced by the T cell bispecific antibody). In some aspects, (administration of) the TKI causes inhibition of the cytotoxic activity of T cells (induced by the T cell bispecific antibody).

“Cytotoxic activity” of a T cell refers to the induction of lysis (i.e. killing) of target cells by a T lymphocyte, particularly a CD4+ or CD8+ T cell. Cytotoxic activity typically involves degranulation of the T lymphocyte, associated with the release of cytotoxic effector molecules such as granzyme B and/or perforin from the T lymphocyte.

In some aspects, (administration of) the TKI causes inhibition of T cell receptor signaling in T cells (induced by the T cell bispecific antibody).

By “T cell receptor signaling” is meant activity of the signaling pathway downstream of the T cell receptor (TCR) in a T lymphocyte following engagement of the TCR (such as engagement of the CD3ε subunit of the TCR by a T cell bispecific antibody), involving signaling molecules including tyrosine kinases such as Lck kinase.

In some aspects, (administration of) the TKI causes inhibition of cytokine secretion by T cells (induced by the T cell bispecific antibody). In some aspects, said cytokine is one or more cytokine selected from the group consisting of IL-2, TNF-α, IFN-γ, IL-6 and IL-1β. In some aspects, said T cells are CD8+ T cells or CD4+ cells.

In some aspects, said inhibition is reversible (i.e. said inhibition can be undone, such that the level of the inhibited parameter returns to about the level it had before the inhibition). In some aspects, said inhibition is reversed after the TKI has not been administered (to the individual) for a given period of time (i.e. after the administration of the TKI is stopped). In some aspects, said period of time is about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours.

Said inhibition may be partial or complete. In some aspects, said inhibition is clinically meaningful and/or statistically significant.

In some aspects, (administration of) the TKI causes reduction of the serum level of one of more cytokine in the individual. In some aspects, said one or more cytokine is selected from the group consisting of IL-2, TNF-α, IFN-γ, IL-6 and IL-1β. In some aspects, said reduction is sustained after the TKI has not been administered (to the individual) for a given amount of time. In some aspects, said amount of time is about 1 hour, 2 hours, 3 hours, 4 hours, 5 hours, 6 hours, 7 hours, 8 hours, 12 hours, 16 hours, 20 hours, 24 hours, 36 hours, 48 hours, 72 hours, or 96 hours. In some aspects, said reduction is sustained after a subsequent administration of the T cell bispecific antibody. Particularly, said reduction is sustained even after administration of the TKI is stopped/no further administration of the TKI is made. Said reduction of the serum level is in particular as compared to the serum level in an individual (including the same individual) without administration of the TKI (i.e. in such case the serum level is reduced as compared to the serum level without/before administration of the TKI). Said reduction of the serum level is in particular as compared to the serum level in an individual (including the same individual) with administration (in particular first administration) of the T cell bispecific antibody but without administration of the TKI (i.e. in such case the serum level is reduced as compared to the serum level with/after administration of the T cell bispecific antibody but without/before administration of the TKI). Without said reduction, the serum level and/or cytokine secretion particularly may be elevated/increased in relation to the (administration of) the T cell bispecific antibody. In some aspects, said reduction is clinically meaningful and/or statistically significant.

In some aspects, said adverse effect is cytokine release syndrome (CRS).

An “adverse effect”, which is sometimes also denoted as “side effect” or “adverse event” (especially in clinical studies is a harmful and undesired effect resulting from medication in the treatment of an individual, herein particularly with a T cell bispecific antibody.

“Cytokine release syndrome” (abbreviated as “CRS”) refers to an increase in the levels of cytokines, such tumor necrosis factor alpha (TNF-α), interferon gamma (IFN-γ), interleukin-6 (IL-6), interleukin-2 (IL-2) and others, in the blood of a subject during or shortly after (e.g. within 1 day of) administration of a therapeutic agent (e.g. a T cell bispecific antibody), resulting in adverse symptoms. CRS is an adverse reaction to therapeutic agent and timely related to administration of the therapeutic agent. It typically occurs during or shortly after an administration of the therapeutic agent, i.e. typically within 24 hours after administration (typically infusion), predominantly at the first administration. In some instances, e.g. after the administration of CAR-T cells, CRS can also occur only later, e.g. several days after administration upon expansion of the CAR-T cells. The incidence and severity typically decrease with subsequent administrations. Symptoms may range from symptomatic discomfort to fatal events, and may include fever, chills, dizziness, hypertension, hypotension, hypoxia, dyspnea, restlessness, sweating, flushing, skin rash, tachycardia, tachypnoea, headache, tumour pain, nausea, vomiting and/or organ failure.

In some aspects, said adverse effect is fever, hypotension and/or hypoxia. In some aspect, said adverse effect is an elevated serum level of one of more cytokine. Said elevated serum level is in particular as compared to the serum level in a healthy individual, and/or the serum level in an individual (including the same individual) without administration of the T cell bispecific antibody (i.e. in such case the serum level is elevated as compared to the serum level without administration of the T cell bispecific antibody). In some aspects, said one or more cytokine is selected from the group consisting of IL-2, TNF-α, IFN-γ, IL-6 and IL-1β.

In some aspects, said adverse effect is an adverse effect related to binding of the T cell bispecific antibody to non-cancer cells expressing the target cell antigen of the T cell bispecific antibody (i.e. an on-target/off-tumor effect). Non-cancer cells may be normal (i.e. not cancerous) cells and/or cells in healthy tissue (i.e. outside of a tumor). In some aspects, said adverse effect is an adverse effect unrelated to binding of the T cell bispecific antibody to its target cell antigen (i.e. an off-target effect).

In some aspects, administration of the TKI is upon (clinical) manifestation of the adverse effect (in the individual). Said administration may be, for example, within about 1 hour, 2 hours, 3 hours, 4 hours, 5 hours, 6 hours, 7 hours, 8 hours, 12 hours, 16 hours, 20 hours or 24 hours after manifestation of the adverse effect (i.e. the occurrence clinical symptoms of the side effect, such as fever). In some aspects, administration of the TKI is in response to the (clinical) manifestation of the adverse effect (in the individual).

In some aspects, administration of the TKI is before the administration of the T cell bispecific antibody. In some aspects, administration of the TKI is concurrent to the administration of the T cell bispecific antibody. In some aspects, administration of the TKI is after the administration of the T cell bispecific antibody. Where administration of the TKI is before or after the administration of the T cell bispecific antibody, such administration of the TKI may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the T cell bispecific antibody. Administration of the TKI may be intermittently or continuously. In some aspects, administration of the TKI is oral.

In some aspects, administration of the TKI is at a dose sufficient to cause inhibition of the activity of the T cell bispecific antibody. In some aspects, administration of the TKI is at a dose sufficient to cause inhibition of the activation of T cells (induced by the T cell bispecific antibody). In some aspects, administration of the TKI is at a dose sufficient to cause inhibition of the proliferation of T cells (induced by the T cell bispecific antibody). In some aspects, administration of the TKI is at a dose sufficient to cause inhibition of the cytotoxic activity of T cells (induced by the T cell bispecific antibody). In some aspects, administration of the TKI is at a dose sufficient to cause inhibition of T cell receptor signaling in T cells (induced by the T cell bispecific antibody). In some aspects, administration of the TKI is at a dose sufficient to cause inhibition of cytokine secretion by T cells (induced by the T cell bispecific antibody). In some aspects, said cytokine is one or more cytokine selected from the group consisting of IL-2, TNF-α, IFN-γ, IL-6 and IL-1β. In some aspects, said T cells are CD8+ T cells or CD4+ cells. Said inhibition may be partial or complete. In some aspects, said inhibition is clinically meaningful and/or statistically significant.

In some aspects, administration of the TKI is at a dose sufficient to cause reduction of the serum level of one of more cytokine in the individual. In some aspects, administration of the TKI is at a dose sufficient to cause reduction of the serum level of one of more cytokine in the individual but insufficient to cause inhibition of the activity of the T cell bispecific antibody. In some aspects, administration of the TKI is at a dose sufficient to cause reduction of the secretion of one of more cytokine by immune cells in the individual but insufficient to cause inhibition of the activation of T cells and/or the cytotoxic activity of T cells induced by the T cell bispecific antibody. In some aspects, said one or more cytokine is selected from the group consisting of IL-2, TNF-α, IFN-γ, IL-6 and IL-1β. In some aspects, said T cells are are CD8+ T cells or CD4+ cells. Immune cells may include various immune cell types, such as T cells, macrophages, NK cells etc.

Said reduction of the serum level or cytokine secretion is in particular as compared to the serum level or cytokine secretion in an individual (including the same individual) without administration of the TKI (i.e. in such case the serum level is reduced as compared to the serum level without/before administration of the TKI). Said reduction of the serum level or cytokine secretion is in particular as compared to the serum level or cytokine secretion in an individual (including the same individual) with administration (in particular first administration) of the T cell bispecific antibody but without administration of the TKI (i.e. in such case the serum level is reduced as compared to the serum level with/after administration of the T cell bispecific antibody but without/before administration of the TKI). Without said reduction, the serum level and/or cytokine secretion particularly may be elevated/increased in relation to the (administration of) the T cell bispecific antibody. In some aspects, said reduction is clinically meaningful and/or statistically significant. Said inhibition may be partial or complete. In some aspects, said inhibition is clinically meaningful and/or statistically significant.

In some aspects, administration of the TKI is at an effective dose.

An “effective amount” or “effective dose” of an agent, e.g. a TKI or a T cell bispecific antibody, refers to an amount effective, at dosages and for periods of time necessary, to achieve the desired therapeutic or prophylactic result.

In some aspects, administration of the TKI is at a dose of about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some aspects, administration of the TKI is at a dose of about 20 mg. In some aspects, administration of the TKI is at a dose of about 70 mg. In some aspects, administration of the TKI is at a dose of about 80 mg. In some aspects, administration of the TKI is at a dose of about 100 mg. In some aspects, administration of the TKI is at a dose of about 140 mg.

In some aspects, administration of the TKI is at a dose of about 100 mg or lower. In some aspects, administration of the TKI is at a dose of about 20 mg. In some aspects, administration of the TKI is at a dose of about 70 mg. In some aspects, administration of the TKI is at a dose of about 80 mg.

In some aspects, administration of the TKI is at a dose of about 100 mg. In such aspects, the dose of the TKI may be sufficient to cause reduction of the serum level of one of more cytokine in the individual but insufficient to cause inhibition of the activity of the T cell bispecific antibody, or sufficient to cause reduction of the secretion of one of more cytokine by immune cells in the individual but insufficient to cause inhibition of the activation of T cells and/or the cytotoxic activity of T cells induced by the T cell bispecific antibody.

In some aspects, administration of the TKI is daily. In some aspects, administration of the TKI is once daily. In some aspects, administration of the TKI is once daily at a dose of about 100 mg. In some aspects, administration of the TKI is for the period of time during which the adverse effect persists (i.e. administration of the TKI is from manifestation of the adverse effect until reduction or disappearance of the adverse effect). In some aspects, administration of the TKI is stopped after the adverse effect is prevented or mitigated. In some aspects, administration of the TKI is stopped after reduction or disappearance of the adverse effect. Said reduction particularly is clinically meaningful and/or statistically significant. In some aspects, administration of the TKI is once, twice, three times, four times, five times, six times, seven times, eight times, nine times or ten times, particularly once, twice, three times, four times, five times, six times, seven times, eight times, nine times or ten times in the course of the treatment of the individual with the T cell bispecific antibody. In some aspects, administration of the TKI is for 1 day, 2 days, 3 days, 4 days, 5 days, 6 days, 7 days, 8 days, 9 days or 10 days. In some aspects, administration of the TKI is once daily for 1 day, 2 days, 3 days, 4 days, 5 days, 6 days, 7 days, 8 days, 9 days or 10 days. In some aspects, administration of the TKI is associated with the first administration of the T cell bispecific antibody. Said first administration is particularly the first administration of the T cell bispecific antibody in the course of the treatment of the individual with the T cell bispecific antibody. In some aspects, administration of the TKI is concurrent with the first administration of the T cell bispecific antibody. In some aspects, administration of the TKI is prior to the first administration of the T cell bispecific antibody. In some aspects, administration of the TKI is subsequent to the first administration of the T cell bispecific antibody. In some aspects, administration of the TKI is subsequent to the first administration of the T cell bispecific antibody and prior to a second administration of the T cell bispecific antibody. Where administration of the TKI is prior or subsequent to the (first) administration of the T cell bispecific antibody, such administration of the TKI may be, for example, within about 1 hour, 2 hours, 3 hours, 4 hours, 5 hours, 6 hours, 7 hours, 8 hours, 12 hours, 16 hours, 20 hours or 24 hours before or after, respectively, the administration of the T cell bispecific antibody.

In some aspects, the administration of the T cell bispecific antibody is for a longer period of time than the administration of the TKI. In some aspects, the administration of the T cell bispecific antibody continues after the administration of the TKI is stopped. In some aspects, the administration of the T cell bispecific antibody is a single administration or a repeated administration. In the course of the treatment of the individual with the T cell bispecific antibody, the T cell bispecific antibody may be administered once or several times. For example, treatment of the individual with the T cell bispecific antibody may comprise multiple treatment cycles which each comprise one or more administrations of the T cell bispecific antibody. In some aspects, the administration of the T cell bispecific antibody comprises a first and a second administration.

For use in the present invention, the T cell bispecific antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some aspects, the administration of the T cell bispecific antibody is at an effective dose. For systemic administration, an effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. Dosage amount and interval may be adjusted individually to provide plasma levels of the T cell bispecific antibody which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

An effective amount of the T cell bispecific antibody may be administered for prevention or treatment of disease. The appropriate route of administration and dosage of the T cell bispecific antibody may be determined based on the type of disease to be treated, the type of the T cell bispecific antibody, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The T cell bispecific antibody and the TKI can be administered by any suitable route, and may be administered by the same route of administration or by different routes of administration. In some aspects, the administration of the T cell bispecific antibody is parenteral, particularly intravenous.

In some aspects, the administration of the T cell bispecific antibody is the first administration of the T cell bispecific antibody to the individual, particularly the first administration of the T cell bispecific antibody in the course of the treatment of the individual with the T cell bispecific antibody.

In some aspects, (administration of) the T cell bispecific antibody induces (i.e. causes or increases) the activation of T cells. In some aspects, (administration of) the T cell bispecific antibody induces the proliferation of T cells. In some aspects, (administration of) the T cell bispecific antibody induces cytotoxic activity of T cells. In some aspects, (administration of) the T cell bispecific antibody induces T cell receptor signaling in T cells. In some aspects, (administration of) the T cell bispecific antibody induces cytokine secretion by T cells. In some aspects, cytokine is one or more cytokine selected from the group consisting of IL-2, TNF-α, IFN-γ, IL-6 and IL-1β. In some aspects, said T cells are CD8+ T cells or CD4+ cells.

In some aspects, administration of the T cell bispecific antibody results in activation of T cells, particularly cytotoxic T cells, particularly at the site of the cancer (e.g. within a solid tumor cancer). Said activation may comprise proliferation of T cells, differentiation of T cells, cytokine secretion by T cells, cytotoxic effector molecule release from T cells, cytotoxic activity of T cells, and expression of activation markers by T cells. In some aspects, the administration of the T cell bispecific antibody results in an increase of T cell, particularly cytotoxic T cell, numbers at the site of the cancer (e.g. within a solid tumor cancer).

In the following, the T cell bispecific antibody that may be used in the present invention is described.

By “T cell bispecific antibody” is meant an antibody that is able to bind, including simultaneously bind, to a T cell (typically via an antigenic determinant expressed on the T cell, such as CD3) and to a target cell (typically via an antigenic determinant expressed on the target cell, such as CEA, CD19, CD20 or HLA-A2/WT1).

In preferred aspects according to the invention, the T cell bispecific antibody is capable of simultaneous binding to the antigenic determinant on the T cell (i.e. a first antigen such as CD3) and the antigenic determinant on the target cell (i.e. a second antigen such as CEA, CD19, CD20 or HLA-A2/WT1). In some aspects, the T cell bispecific antibody is capable of crosslinking the T cell and the target cell by simultaneous binding to CD3 and a target cell antigen. In even more preferred aspects, such simultaneous binding results in lysis of the target cell, particularly a target cell antigen (e.g. CEA, CD19, CD20 or HLA-A2/WT1)-expressing tumor cell. In some aspects, such simultaneous binding results in activation of the T cell. In some aspects, such simultaneous binding results in a cellular response of the T cell, selected from the group of: proliferation, differentiation, cytokine secretion, cytotoxic effector molecule release, cytotoxic activity, and expression of activation markers. In some aspects, binding of the T cell bispecific antibody to CD3 without simultaneous binding to the target cell antigen does not result in T cell activation. In some aspects, the T cell bispecific antibody is capable of re-directing cytotoxic activity of a T cell to a target cell. In preferred aspects, said re-direction is independent of MHC-mediated peptide antigen presentation by the target cell and and/or specificity of the T cell.

The term “bispecific” means that the antibody is able to bind to at least two distinct antigenic determinants. Typically, a bispecific antibody comprises two antigen binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that binds, including specifically binds, to an antigenic determinant. In some aspects, an antigen binding moiety is able to direct the entity to which it is attached (e.g. a second antigen binding moiety) to a target site, for example to a specific type of tumor cell bearing the antigenic determinant. In further aspects, an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The term “bind” or “binding” herein generally refers to “specific binding”. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In some aspects, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In some aspects, CD3 is human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 25. The amino acid sequence of cynomolgus [Macaca fascicularis] CD3ε is shown in NCBI GenBank no. BAB71849.1. See also SEQ ID NO: 26.

A “target cell antigen” as used herein refers to an antigenic determinant presented on the surface of a target cell, for example a cell in a tumor such as a cancer cell or a cell of the tumor stroma (in that case a “tumor cell antigen”). Preferably, the target cell antigen is not CD3, and/or is expressed on a different cell than CD3. In some aspects, the target cell antigen is CEA, particularly human CEA. In other aspects, the target cell antigen is HLA-A2/WT1, particularly human HLA-A2/WT1. In some aspects, the target cell antigen is CD20, particularly human CD20. In some aspects, the target cell antigen is CD19, particularly human CD19.

As used herein, the terms “first”, “second” or “third” with respect to antigen binding moieties etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, Md. (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and

FR4. Accordingly, the HVR and FR sequences generally appear in the following order in VH (or VL): FR1-H1(L1)-FR2-H2(L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some aspects the modification promoting association comprises an amino acid mutation in the Fc domain, specifically an amino acid substitution. In particular aspects, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

In particular aspects, the T cell bispecific antibody binds to CD3 and a target cell antigen. Accordingly, in some aspects, the T cell bispecific antibody comprises an antigen binding moiety that binds to CD3 and an antigen binding moiety that binds to a target cell antigen.

In some aspects, the first and/or the second antigen binding moiety is a Fab molecule. In some aspects, the first antigen binding moiety is a crossover Fab molecule wherein either the variable or the constant regions of the Fab light chain and the Fab heavy chain are exchanged. In such aspects, the second antigen binding moiety preferably is a conventional Fab molecule.

In some aspects wherein the first and the second antigen binding moiety of the T cell bispecific antibody are both Fab molecules, and in one of the antigen binding moieties (particularly the first antigen binding moiety) the variable domains VL and VH of the Fab light chain and the Fab heavy chain are replaced by each other,

i) in the constant domain CL of the first antigen binding moiety the amino acid at position 124 is substituted by a positively charged amino acid (numbering according to Kabat), and wherein in the constant domain CH1 of the first antigen binding moiety the amino acid at position 147 or the amino acid at position 213 is substituted by a negatively charged amino acid (numbering according to Kabat EU index); or
ii) in the constant domain CL of the second antigen binding moiety the amino acid at position 124 is substituted by a positively charged amino acid (numbering according to Kabat), and wherein in the constant domain CH1 of the second antigen binding moiety the amino acid at position 147 or the amino acid at position 213 is substituted by a negatively charged amino acid (numbering according to Kabat EU index).

The T cell bispecific antibody does not comprise both modifications mentioned under i) and ii). The constant domains CL and CH1 of the antigen binding moiety having the VH/VL exchange are not replaced by each other (i.e. remain unexchanged).

In more specific aspects,

i) in the constant domain CL of the first antigen binding moiety the amino acid at position 124 is substituted independently by lysine (K), arginine (R) or histidine (H) (numbering according to Kabat), and in the constant domain CH1 of the first antigen binding moiety the amino acid at position 147 or the amino acid at position 213 is substituted independently by glutamic acid (E), or aspartic acid (D) (numbering according to Kabat EU index); or
ii) in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In some aspects, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In further aspects, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index).

In preferred aspects, in the constant domain CL of the second antigen binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In some aspects, in the constant domain CL of the second antigen binding moiety the amino acid at position 124 is substituted by lysine (K) (numbering according to Kabat) and the amino acid at position 123 is substituted by lysine (K) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In some aspects, in the constant domain CL of the second antigen binding moiety the amino acid at position 124 is substituted by lysine (K) (numbering according to Kabat) and the amino acid at position 123 is substituted by arginine (R) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second antigen binding moiety, the constant domain CL of the second antigen binding moiety is of kappa isotype.

In some aspects, the first and the second antigen binding moiety are fused to each other, optionally via a peptide linker.

In some aspects, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some aspects, the T cell bispecific antibody provides monovalent binding to CD3.

In particular aspects, the T cell bispecific antibody comprises a single antigen binding moiety that binds to CD3, and two antigen binding moieties that bind to the target cell antigen. Thus, in some aspects, the T cell bispecific antibody comprises a third antigen binding moiety, particularly a Fab molecule, more particularly a conventional Fab molecule, that binds to the target antigen. The third antigen binding moiety may incorporate, singly or in combination, all of the features described herein in relation to the second antigen binding moiety (e.g. the CDR sequences, variable region sequences, and/or amino acid substitutions in the constant regions). In some aspects, the third antigen moiety is identical to the first antigen binding moiety (e.g. is also a conventional Fab molecule and comprises the same amino acid sequences).

In particular aspects, the T cell bispecific antibody further comprises an Fc domain composed of a first and a second subunit. In some aspects, the Fc domain is an IgG Fc domain. In particular aspects, the Fc domain is an IgG₁ Fc domain. In other aspects, the Fc domain is an IgG₄ Fc domain. In more specific aspects, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In further particular aspects, the Fc domain is a human Fc domain. In particularly preferred aspects, the Fc domain is a human IgG₁ Fc domain. An exemplary sequence of a human IgG₁ Fc region is given in SEQ ID NO: 27.

In some aspects wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In some aspects, the T cell bispecific antibody essentially consists of the first, the second and the third antigen binding moiety (particularly Fab molecule), the Fc domain composed of a first and a second subunit, and optionally one or more peptide linkers.

The components of the T cell bispecific antibody may be fused to each other directly or, preferably, via one or more suitable peptide linkers. Where fusion of a Fab molecule is to the N-terminus of a subunit of the Fc domain, it is typically via an immunoglobulin hinge region.

The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ, G₄(SG₄)ₙ or (G₄S)ₙG₅ peptide linkers. “n” is generally an integer from 1 to 10, typically from 2 to 4. In some aspects, said peptide linker has a length of at least 5 amino acids, in some aspects a length of 5 to 100, in further aspects of 10 to 50 amino acids. In some aspects said peptide linker is (GxS)ₙ or (GxS)ₙGₘ with G=glycine, S=serine, and (x=3, n=3, 4, 5 or 6, and m=0, 1, 2 or 3) or (x=4, n=1, 2, 3, 4 or 5 and m=0, 1, 2, 3, 4 or 5), in some aspects x=4 and n=2 or 3, in further aspects x=4 and n=2, in yet further aspects x=4, n=1 and m=5. In some aspects, said peptide linker is (G₄S)₂. In other aspects, said peptide linker is G₄SG₅. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in some aspects, said modification is in the CH3 domain of the Fc domain.

In specific aspects, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In specific such aspects, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In further aspects, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In preferred aspects, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In particular aspects, the Fc receptor is an Fcγ receptor. In some aspects, the Fc receptor is a human Fc receptor. In some aspects, the Fc receptor is an activating Fc receptor. In specific aspects, the Fc receptor is an activating human Fcγ receptor, more specifically human FcγRIIIa, FcγRI or FcγRIIa, most specifically human FcγRIIIa. In some aspects, the effector function is one or more selected from the group of complement dependent cytotoxicity (CDC), antibody-dependent cell-mediated cytotoxicity (ADCC), antibody-dependent cellular phagocytosis (ADCP), and cytokine secretion. In particular aspects, the effector function is ADCC.

Typically, the same one or more amino acid substitution is present in each of the two subunits of the Fc domain. In some aspects, the one or more amino acid substitution reduces the binding affinity of the Fc domain to an Fc receptor. In some aspects, the one or more amino acid substitution reduces the binding affinity of the Fc domain to an Fc receptor by at least 2-fold, at least 5-fold, or at least 10-fold.

In some aspects, the Fc domain comprises an amino acid substitution at a position selected from the group of E233, L234, L235, N297, P331 and P329 (numberings according to Kabat EU index). In more specific aspects, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In some such aspects, the Fc domain is an IgG₁ Fc domain, particularly a human IgG₁ Fc domain. In some aspects, the Fc domain comprises an amino acid substitution at position P329. In more specific aspects, the amino acid substitution is P329A or P329G, particularly P329G (numberings according to Kabat EU index). In some aspects, the Fc domain comprises an amino acid substitution at position P329 and a further amino acid substitution at a position selected from E233, L234, L235, N297 and P331 (numberings according to Kabat EU index). In more specific aspects, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In some such aspects, the Fc domain is an IgG₁ Fc domain, particularly a human IgG₁ Fc domain.

In some aspects, the target cell antigen of the T cell bispecific antibody is carcinoembryonic antigen (CEA).

“Carcinoembryonic antigen” or “CEA” (also known as Carcinoembryonic antigen-related cell adhesion molecule 5 (CEACAM5)) refers to any native CEA from any vertebrate source, including mammals such as primates (e.g. humans), non-human primates (e.g. cynomolgus monkeys) and rodents (e.g. mice and rats), unless otherwise indicated. The term encompasses “full-length,” unprocessed CEA as well as any form of CEA that results from processing in the cell. The term also encompasses naturally occurring variants of CEA, e.g., splice variants or allelic variants. In some aspects, CEA is human CEA. The amino acid sequence of human CEA is shown in UniProt (www.uniprot.org) accession no. P06731, or NCBI (www.ncbi.nlm.nih.gov/) RefSeq NP_004354.2. In some aspects, CEA is cell membrane-bound CEA. In some aspects, CEA is CEA expressed on the surface of a cell, e.g. a cancer cell.

Useful T cell bispecific antibodies for the present invention that bind to CEA are described e.g. in PCT publication no. WO 2014/131712 (incorporated herein by reference in its entirety).

Is some aspects, the T cell bispecific antibody comprises a first antigen binding moiety that binds to CD3, and a second antigen binding moiety that binds to CEA.

In some aspects, the first antigen binding moiety comprises a heavy chain variable region comprising the heavy chain CDR (HCDR) 1 of SEQ ID NO: 28, the HCDR2 of SEQ ID NO: 29, and the HCDR3 of SEQ ID NO: 30; and a light chain variable region comprising the light chain CDR (LCDR) 1 of SEQ ID NO: 31, the LCDR2 of SEQ ID NO: 32 and the LCDR3 of SEQ ID NO: 33.

In some aspects, the second antigen binding moiety comprises a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

In some aspects, the CEA CD3 bispecific antibody comprises

(i) a first antigen binding moiety that binds to CD3 and comprises a heavy chain variable region comprising the heavy chain CDR (HCDR) 1 of SEQ ID NO: 28, the HCDR2 of SEQ ID NO: 29, and the HCDR3 of SEQ ID NO: 30; and a light chain variable region comprising the light chain CDR (LCDR) 1 of SEQ ID NO: 31, the LCDR2 of SEQ ID NO: 32 and the LCDR3 of SEQ ID NO: 33; and
(ii) a second antigen binding moiety that binds to CEA and comprises a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41.

In some aspects, the first antigen binding moiety comprises a heavy chain variable region sequence that is at least about 95%, 96%, 97%, 98%, 99% or 100% identical to the amino acid sequence of SEQ ID NO: 34 and a light chain variable region sequence that is at least about 95%, 96%, 97%, 98%, 99% or 100% identical to the amino acid sequence of SEQ ID NO: 35. In some aspects, the first antigen binding moiety comprises the heavy chain variable region sequence of SEQ ID NO: 34 and the light chain variable region sequence of SEQ ID NO: 35.

In some aspects, the second antigen binding moiety comprises a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43. In some aspects, the second antigen binding moiety comprises the heavy chain variable region sequence of SEQ ID NO: 42 and the light chain variable region sequence of SEQ ID NO: 43.

In some aspects, the T cell bispecific antibody comprises a third antigen binding moiety that binds to CEA and/or an Fc domain composed of a first and a second subunit, as described herein.

In preferred aspects, the T cell bispecific antibody comprises

(i) a first antigen binding moiety that binds to CD3, comprising a heavy chain variable region comprising the heavy chain CDR (HCDR) 1 of SEQ ID NO: 28, the HCDR2 of SEQ ID NO: 29, and the HCDR3 of SEQ ID NO: 30; and a light chain variable region comprising the light chain CDR (LCDR) 1 of SEQ ID NO: 31, the LCDR2 of SEQ ID NO: 32 and the LCDR3 of SEQ ID NO: 33, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety that bind to CEA, comprising a heavy chain variable region comprising the heavy chain CDR (HCDR) 1 of SEQ ID NO: 36, the HCDR2 of SEQ ID NO: 37, and the HCDR3 of SEQ ID NO: 38; and a light chain variable region comprising the light chain CDR (LCDR) 1 of SEQ ID NO: 39, the LCDR2 of SEQ ID NO: 40 and the LCDR3 of SEQ ID NO: 41, wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to CEA and CD3) comprises a heavy chain variable region sequence that is at least about 95%, 96%, 97%, 98%, 99% or 100% identical to the amino acid sequence of SEQ ID NO: 34 and a light chain variable region sequence that is at least about 95%, 96%, 97%, 98%, 99% or 100% identical to the amino acid sequence of SEQ ID NO: 35. In some aspects, the first antigen binding moiety comprises the heavy chain variable region sequence of SEQ ID NO: 34 and the light chain variable region sequence of SEQ ID NO: 35.

In some aspects, the second and (where present) third antigen binding moiety of the T cell bispecific antibody (that binds to CEA and CD3) comprise a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43. In some aspects, the second and (where present) third antigen binding moiety comprise the heavy chain variable region of SEQ ID NO: 42 and the light chain variable region of SEQ ID NO: 43.

The Fc domain according to the above aspects may incorporate, singly or in combination, all of the features described hereinabove in relation to Fc domains.

In some aspects, the Fc domain of the T cell bispecific antibody (that binds to CEA and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, particularly by peptide linkers as in SEQ ID NO: 45 and SEQ ID NO: 47.

In some aspects, the T cell bispecific antibody (that binds to CEA and CD3) comprises a polypeptide (particularly two polypeptides) comprising a sequence that is at least 80%, 85%, 90%, 95%, 96%, 97%, 98%, or 99% identical to the sequence of SEQ ID NO: 44, a polypeptide comprising a sequence that is at least 80%, 85%, 90%, 95%, 96%, 97%, 98%, or 99% identical to the sequence of SEQ ID NO: 45, a polypeptide comprising a sequence that is at least 80%, 85%, 90%, 95%, 96%, 97%, 98%, or 99% identical to the sequence of SEQ ID NO: 46, and a polypeptide comprising a sequence that is at least 80%, 85%, 90%, 95%, 96%, 97%, 98%, or 99% identical to the sequence of SEQ ID NO: 47. In some aspects, the T cell bispecific antibody (that binds to CEA and CD3) comprises a polypeptide (particularly two polypeptides) comprising the sequence of SEQ ID NO: 44, a polypeptide comprising the sequence of SEQ ID NO: 45, a polypeptide comprising the sequence of SEQ ID NO: 46, and a polypeptide comprising the sequence of SEQ ID NO: 47.

In preferred aspects, the T cell bispecific antibody is cibisatamab (WHO Drug Information (International Nonproprietary Names for Pharmaceutical Substances), Recommended INN: List 80, 2018, vol. 32, no. 3, p. 438).

In some aspects, the target cell antigen of the T cell bispecific antibody is HLA-2/WT1.

“WT1”, also known as “Wilms tumor 1” or “Wilms tumor protein”, refers to any native WT1 from any vertebrate source, including mammals such as primates (e.g. humans), non-human primates (e.g. cynomolgus monkeys) and rodents (e.g. mice and rats), unless otherwise indicated. The term encompasses “full-length,” unprocessed WT1 as well as any form of WT1 that results from processing in the cell. The term also encompasses naturally occurring variants of WT1, e.g., splice variants or allelic variants. In some aspects, WT1 is human WT1, particularly the protein of SEQ ID NO: 21. Human WT1 is described in UniProt (www.uniprot.org) accession no. P19544 (entry version 215), and an amino acid sequence of human WT1 is also shown in SEQ ID NO: 21.

By “VLD”, “VLD peptide” or “WT1_{VLD}” is meant the WT1 derived peptide having the amino acid sequence VLDFAPPGA (SEQ ID NO: 22; position 37-45 of the WT1 protein of SEQ ID NO: 21).

By “RMF”, “RMF peptide” or “WT1_{RMF}” is meant the WT1 derived peptide having the amino acid sequence RMFRNAPYL (SEQ ID NO: 23; position 126-134 of the WT1 protein of SEQ ID NO: 21).

“HLA-A2”, “HLA-A*02”, “HLA-A02”, or “HLA-A*2” (used interchangeably) refers to a human leukocyte antigen serotype in the HLA-A serotype group. The HLA-A2 protein (encoded by the respective HLA gene) constitutes the α chain of the respective class I MHC (major histocompatibility complex) protein, which further comprises a (32 microglobulin subunit. A specific HLA-A2 protein is HLA-A201 (also referred to as HLA-A0201, HLA-A02.01, or HLA-A*02:01). In specific aspects, the HLA-A2 protein described herein is HLA-A201. An exemplary sequence of human HLA-A2 is given in SEQ ID NO: 24.

“HLA-A2/WT1” refers to a complex of a HLA-A2 molecule and a WT1 derived peptide (also referred to herein as a “WT1 peptide”), specifically the RMF or VLD peptide (“HLA-A2/WT1_{RMF}” and “HLA-A2/WT1_{VLD}”, respectively). The bispecific antibody used in the present invention specifically may bind to either the HLA-A2/WT1_{RMF} or the HLA-A2/WT1_{VLD} complex.

Useful T cell bispecific antibodies for the present invention that bind to HLA-A2/WT1 are described e.g. in PCT publication no. WO 2019/122052 (incorporated herein by reference in its entirety).

In some aspects, the T cell bispecific antibody comprises a first antigen binding moiety that binds to CD3, and a second antigen binding moiety that binds to HLA-A2/WT1, particularly HLA-A2/WT1_{RMF}.

In some aspects, the first antigen binding moiety comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6.

In some aspects, the second antigen binding moiety comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In some aspects, the T cell bispecific antibody comprises

(i) a first antigen binding moiety that binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and
(ii) a second antigen binding moiety that binds to HLA-A2/WT1 and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In some aspects, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8. In some aspects, the first antigen binding moiety comprises the heavy chain variable region sequence of SEQ ID NO: 7 and the light chain variable region sequence of SEQ ID NO: 8.

In some aspects, the second antigen binding moiety comprises a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16. In some aspects, the second antigen binding moiety comprises the heavy chain variable region sequence of SEQ ID NO: 15 and the light chain variable region sequence of SEQ ID NO: 16.

In some aspects, the T cell bispecific antibody comprises a third antigen binding moiety that binds to HLA-A2/WT1 and/or an Fc domain composed of a first and a second subunit, as described herein.

In preferred aspects, the T cell bispecific antibody comprises

(i) a first antigen binding moiety that binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that bind to HLA-A2/WT1, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to HLA-A2/WT1 and CD3) is a crossover Fab molecule wherein the variable regions of the Fab light chain and the Fab heavy chain are exchanged, and wherein the second and (where present) third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above aspects, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

In some aspects, the first antigen binding moiety of the T cell bispecific antibody (that binds to HLA-A2/WT1 and CD3) comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8. In some aspects, the first antigen binding moiety comprises the heavy chain variable region sequence of SEQ ID NO: 7 and the light chain variable region sequence of SEQ ID NO: 8.

In some aspects, the second and (where present) third antigen binding moiety of the T cell bispecific antibody (that binds to HLA-A2/WT1 and CD3) comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16. In some aspects, the second and (where present) third antigen binding moiety comprise the heavy chain variable region of SEQ ID NO: 15 and the light chain variable region of SEQ ID NO: 16.

The Fc domain according to the above aspects may incorporate, singly or in combination, all of the features described hereinabove in relation to Fc domains.

In some aspects, the Fc domain of the T cell bispecific antibody (that binds to HLA-A2/WT1 and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, particularly by peptide linkers as in SEQ ID NO: 18 and SEQ ID NO: 20.

In some aspects, the T cell bispecific antibody (that binds to HLA-A2/WT1 and CD3) comprises a polypeptide (particularly two polypeptides) comprising a sequence that is at least 80%, 85%, 90%, 95%, 96%, 97%, 98%, or 99% identical to the sequence of SEQ ID NO: 17, a polypeptide comprising a sequence that is at least 80%, 85%, 90%, 95%, 96%, 97%, 98%, or 99% identical to the sequence of SEQ ID NO: 18, a polypeptide comprising a sequence that is at least 80%, 85%, 90%, 95%, 96%, 97%, 98%, or 99% identical to the sequence of SEQ ID NO: 19, and a polypeptide comprising a sequence that is at least 80%, 85%, 90%, 95%, 96%, 97%, 98%, or 99% identical to the sequence of SEQ ID NO: 20. In some aspects, the T cell bispecific antibody (that binds to HLA-A2/WT1 and CD3) comprises a polypeptide (particularly two polypeptides) comprising the sequence of SEQ ID NO: 17, a polypeptide comprising the sequence of SEQ ID NO: 18, a polypeptide comprising the sequence of SEQ ID NO: 19, and a polypeptide comprising the sequence of SEQ ID NO: 20.

In some aspects, the target cell antigen of the T cell bispecific antibody is CD20.

“CD20”, also known as “B-lymphocyte antigen B1”, refers to any native CD20 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD20 as well as any form of CD20 that results from processing in the cell. The term also encompasses naturally occurring variants of CD20, e.g., splice variants or allelic variants. In some aspects, CD20 is human CD20. Human CD20 is described in UniProt (www.uniprot.org) accession no. P11836 (entry version 200), and an amino acid sequence of human CD20 is also shown in SEQ ID NO: 60.

Useful T cell bispecific antibodies for the present invention that bind to CD20 are described e.g. in PCT publication no. WO 2016/020309 (incorporated herein by reference in its entirety).

In some aspects, the T cell bispecific antibody comprises a first antigen binding moiety that binds to CD3, and a second antigen binding moiety that binds to CD20.

In some aspects, the first antigen binding moiety comprises a heavy chain variable region comprising the heavy chain CDR (HCDR) 1 of SEQ ID NO: 28, the HCDR2 of SEQ ID NO: 29, and the HCDR3 of SEQ ID NO: 30; and a light chain variable region comprising the light chain CDR (LCDR) 1 of SEQ ID NO: 31, the LCDR2 of SEQ ID NO: 32 and the LCDR3 of SEQ ID NO: 33.

In some aspects, the second antigen binding moiety comprises a heavy chain variable region comprising the heavy chain CDR (HCDR) 1 of SEQ ID NO: 48, the HCDR2 of SEQ ID NO: 49, and the HCDR3 of SEQ ID NO: 50; and a light chain variable region comprising the light chain CDR (LCDR) 1 of SEQ ID NO: 51, the LCDR2 of SEQ ID NO: 52 and the LCDR3 of SEQ ID NO: 53.

In some aspects, the T cell bispecific antibody comprises

(i) a first antigen binding moiety that binds to CD3 and comprises a heavy chain variable region comprising the heavy chain CDR (HCDR) 1 of SEQ ID NO: 28, the HCDR2 of SEQ ID NO: 29, and the HCDR3 of SEQ ID NO: 30; and a light chain variable region comprising the light chain CDR (LCDR) 1 of SEQ ID NO: 31, the LCDR2 of SEQ ID NO: 32 and the LCDR3 of SEQ ID NO: 33; and
(ii) a second antigen binding moiety that binds to CD20 and comprises a heavy chain variable region comprising the heavy chain CDR (HCDR) 1 of SEQ ID NO: 48, the HCDR2 of SEQ ID NO: 49, and the HCDR3 of SEQ ID NO: 50; and a light chain variable region comprising the light chain CDR (LCDR) 1 of SEQ ID NO: 51, the LCDR2 of SEQ ID NO: 52 and the LCDR3 of SEQ ID NO: 53.

In some aspects, the first antigen binding moiety comprises a heavy chain variable region sequence that is at least about 95%, 96%, 97%, 98%, 99% or 100% identical to the amino acid sequence of SEQ ID NO: 34 and a light chain variable region sequence that is at least about 95%, 96%, 97%, 98%, 99% or 100% identical to the amino acid sequence of SEQ ID NO: 35. In some aspects, the first antigen binding moiety comprises the heavy chain variable region sequence of SEQ ID NO: 34 and the light chain variable region sequence of SEQ ID NO: 35.

In some aspects, the second antigen binding moiety comprises a heavy chain variable region sequence that is at least about 95%, 96%, 97%, 98%, 99% or 100% identical to the amino acid sequence of SEQ ID NO: 54 and a light chain variable region sequence that is at least about 95%, 96%, 97%, 98%, 99% or 100% identical to the amino acid sequence of SEQ ID NO: 55. In some aspects, the second antigen binding moiety comprises the heavy chain variable region sequence of SEQ ID NO: 54 and the light chain variable region sequence of SEQ ID NO: 55.

In some aspects, the T cell bispecific antibody comprises a third antigen binding moiety that binds to CD20 and/or an Fc domain composed of a first and a second subunit, as described herein.

In preferred aspects, the T cell bispecific antibody comprises

(i) a first antigen binding moiety that binds to CD3, comprising a heavy chain variable region comprising the heavy chain CDR (HCDR) 1 of SEQ ID NO: 28, the HCDR2 of SEQ ID NO: 29, and the HCDR3 of SEQ ID NO: 30; and a light chain variable region comprising the light chain CDR (LCDR) 1 of SEQ ID NO: 31, the LCDR2 of SEQ ID NO: 32 and the LCDR3 of SEQ ID NO: 33, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that bind to CD20, comprising a heavy chain variable region comprising the heavy chain CDR (HCDR) 1 of SEQ ID NO: 48, the HCDR2 of SEQ ID NO: 49, and the HCDR3 of SEQ ID NO: 50; and a light chain variable region comprising the light chain CDR (LCDR) 1 of SEQ ID NO: 51, the LCDR2 of SEQ ID NO: 52 and the LCDR3 of SEQ ID NO: 53, wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to CD20 and CD3) is a crossover Fab molecule wherein the variable regions of the Fab light chain and the Fab heavy chain are exchanged, and wherein the second and (where present) third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above aspects, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

In some aspects, the first antigen binding moiety of the T cell bispecific antibody (that binds to CD20 and CD3) comprises a heavy chain variable region sequence that is at least about 95%, 96%, 97%, 98%, 99% or 100% identical to the amino acid sequence of SEQ ID NO: 34 and a light chain variable region sequence that is at least about 95%, 96%, 97%, 98%, 99% or 100% identical to the amino acid sequence of SEQ ID NO: 35. In some aspects, the first antigen binding moiety comprises the heavy chain variable region sequence of SEQ ID NO: 34 and the light chain variable region sequence of SEQ ID NO: 35.

In some aspects, the second and (where present) third antigen binding moiety of the T cell bispecific antibody (that binds to CD20 and CD3) comprise a heavy chain variable region sequence that is at least about 95%, 96%, 97%, 98%, 99% or 100% identical to the amino acid sequence of SEQ ID NO: 54 and a light chain variable region sequence that is at least about 95%, 96%, 97%, 98%, 99% or 100% identical to the amino acid sequence of SEQ ID NO: 55. In some aspects, the second and (where present) third antigen binding moiety comprise the heavy chain variable region of SEQ ID NO: 54 and the light chain variable region of SEQ ID NO: 55.

The Fc domain according to the above aspects may incorporate, singly or in combination, all of the features described hereinabove in relation to Fc domains.

In some aspects, the Fc domain of the T cell bispecific antibody (that binds to CD20 and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, particularly by peptide linkers as in SEQ ID NO: 57 and SEQ ID NO: 59.

In some aspects, the T cell bispecific antibody (that binds to CD20 and CD3) comprises a polypeptide (particularly two polypeptides) comprising a sequence that is at least 80%, 85%, 90%, 95%, 96%, 97%, 98%, or 99% identical to the sequence of SEQ ID NO: 56, a polypeptide comprising a sequence that is at least 80%, 85%, 90%, 95%, 96%, 97%, 98%, or 99% identical to the sequence of SEQ ID NO: 57, a polypeptide comprising a sequence that is at least 80%, 85%, 90%, 95%, 96%, 97%, 98%, or 99% identical to the sequence of SEQ ID NO: 58, and a polypeptide comprising a sequence that is at least 80%, 85%, 90%, 95%, 96%, 97%, 98%, or 99% identical to the sequence of SEQ ID NO: 59. In some aspects, the T cell bispecific antibody (that binds to CD20 and CD3) comprises a polypeptide (particularly two polypeptides) comprising the sequence of SEQ ID NO: 56, a polypeptide comprising the sequence of SEQ ID NO: 57, a polypeptide comprising the sequence of SEQ ID NO: 58, and a polypeptide comprising the sequence of SEQ ID NO: 59.

In preferred aspects, the T cell bispecific antibody is glofitamab (WHO Drug Information (International Nonproprietary Names for Pharmaceutical Substances), Recommended INN: List 83, 2020, vol. 34, no. 1, p. 39).

In some aspects, the target cell antigen of the T cell bispecific antibody is CD19.

“CD19” stands for cluster of differentiation 19 (also known as B-lymphocyte antigen CD19 or B-lymphocyte surface antigen B4) and refers to any native CD19 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD19 as well as any form of CD19 that results from processing in the cell. The term also encompasses naturally occurring variants of CD19, e.g., splice variants or allelic variants. In some aspects, CD19 is human CD19. See for the human protein UniProt (www.uniprot.org) accession no. P15391 (version 211), or NCBI (www.ncbi.nlm.nih.gov/) RefSeq NP_001761.3. An exemplary sequence of human CD19 is given in SEQ ID NO: 81.

Useful T cell bispecific antibodies for the present invention that bind to CD19 are described e.g. in EP application nos. 20181056.1 and 20180968.8 (incorporated herein by reference in their entirety).

In some aspects, the T cell bispecific antibody comprises a first antigen binding moiety that binds to CD3, and a second antigen binding moiety that binds to CD19.

In some aspects, the first antigen binding moiety comprises a heavy chain variable region comprising the heavy chain CDR (HCDR) 1 of SEQ ID NO: 61, the HCDR2 of SEQ ID NO: 29, and the HCDR3 of SEQ ID NO: 62; and a light chain variable region comprising the light chain CDR (LCDR) 1 of SEQ ID NO: 31, the LCDR2 of SEQ ID NO: 32 and the LCDR3 of SEQ ID NO: 33.

In other aspects, the first antigen binding moiety comprises a heavy chain variable region comprising the heavy chain CDR (HCDR) 1 of SEQ ID NO: 64, the HCDR2 of SEQ ID NO: 29, and the HCDR3 of SEQ ID NO: 65; and a light chain variable region comprising the light chain CDR (LCDR) 1 of SEQ ID NO: 31, the LCDR2 of SEQ ID NO: 32 and the LCDR3 of SEQ ID NO: 33.

In some aspects, the second antigen binding moiety comprises a heavy chain variable region comprising the heavy chain CDR (HCDR) 1 of SEQ ID NO: 67, the HCDR2 of SEQ ID NO: 68, and the HCDR3 of SEQ ID NO: 69; and a light chain variable region comprising the light chain CDR (LCDR) 1 of SEQ ID NO: 70, the LCDR2 of SEQ ID NO: 71 and the LCDR3 of SEQ ID NO: 72.

In some aspects, the T cell bispecific antibody comprises

(i) a first antigen binding moiety that binds to CD3 and comprises a heavy chain variable region comprising the heavy chain CDR (HCDR) 1 of SEQ ID NO: 61, the HCDR2 of SEQ ID NO: 29, and the HCDR3 of SEQ ID NO: 62, or a heavy chain variable region comprising the HCDR1 of SEQ ID NO: 64, the HCDR2 of SEQ ID NO: 29, and the HCDR3 of SEQ ID NO: 65; and a light chain variable region comprising the light chain CDR (LCDR) 1 of SEQ ID NO: 31, the LCDR2 of SEQ ID NO: 32 and the LCDR3 of SEQ ID NO: 33; and
(ii) a second antigen binding moiety that binds to CD19 and comprises a heavy chain variable region comprising the heavy chain CDR (HCDR) 1 of SEQ ID NO: 67, the HCDR2 of SEQ ID NO: 68, and the HCDR3 of SEQ ID NO: 69; and a light chain variable region comprising the light chain CDR (LCDR) 1 of SEQ ID NO: 70, the LCDR2 of SEQ ID NO: 71 and the LCDR3 of SEQ ID NO: 72.

In some aspects, the first antigen binding moiety comprises a heavy chain variable region sequence that is at least about 95%, 96%, 97%, 98%, 99% or 100% identical to the amino acid sequence of SEQ ID NO: 63 or a heavy chain variable region sequence that is at least about 95%, 96%, 97%, 98%, 99% or 100% identical to the amino acid sequence of SEQ ID NO: 66, and a light chain variable region sequence that is at least about 95%, 96%, 97%, 98%, 99% or 100% identical to the amino acid sequence of SEQ ID NO: 35. In some aspects, the first antigen binding moiety comprises the heavy chain variable region sequence of SEQ ID NO: 63 or the heavy chain variable region sequence of SEQ ID NO: 66, and the light chain variable region sequence of SEQ ID NO: 35.

In some aspects, the second antigen binding moiety comprises a heavy chain variable region sequence that is at least about 95%, 96%, 97%, 98%, 99% or 100% identical to the amino acid sequence of SEQ ID NO: 73 and a light chain variable region sequence that is at least about 95%, 96%, 97%, 98%, 99% or 100% identical to the amino acid sequence of SEQ ID NO: 74. In some aspects, the second antigen binding moiety comprises the heavy chain variable region sequence of SEQ ID NO: 73 and the light chain variable region sequence of SEQ ID NO: 74.

In some aspects, the T cell bispecific antibody comprises a third antigen binding moiety that binds to CD19 and/or an Fc domain composed of a first and a second subunit, as described herein.

In preferred aspects, the T cell bispecific antibody comprises

(i) a first antigen binding moiety that binds to CD3, comprising a heavy chain variable region comprising the heavy chain CDR (HCDR) 1 of SEQ ID NO: 61, the HCDR2 of SEQ ID NO: 29, and the HCDR3 of SEQ ID NO: 62, or a heavy chain variable region comprising the HCDR1 of SEQ ID NO: 64, the HCDR2 of SEQ ID NO: 29, and the HCDR3 of SEQ ID NO: 65; and a light chain variable region comprising the light chain CDR (LCDR) 1 of SEQ ID NO: 31, the LCDR2 of SEQ ID NO: 32 and the LCDR3 of SEQ ID NO: 33, wherein the first antigen binding moiety is a crossover Fab molecule wherein either the variable or the constant regions, particularly the variable regions, of the Fab light chain and the Fab heavy chain are exchanged;
(ii) a second and a third antigen binding moiety that bind to CD19, comprising a heavy chain variable region comprising the heavy chain CDR (HCDR) 1 of SEQ ID NO: 67, the HCDR2 of SEQ ID NO: 68, and the HCDR3 of SEQ ID NO: 69; and a light chain variable region comprising the light chain CDR (LCDR) 1 of SEQ ID NO: 70, the LCDR2 of SEQ ID NO: 71 and the LCDR3 of SEQ ID NO: 72, wherein the second and third antigen binding moiety are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In some aspects, the first antigen binding moiety of the T cell bispecific antibody (that binds to CD19 and CD3) is a crossover Fab molecule wherein the variable regions of the Fab light chain and the Fab heavy chain are exchanged, and wherein the second and (where present) third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

Particularly, in the above aspects, in the constant domain CL of the second and the third Fab molecule under (ii) the amino acid at position 124 may be substituted by lysine (K) (numbering according to Kabat) and the amino acid at position 123 may be substituted by lysine (K) or arginine (R), particularly by arginine (R) (numbering according to Kabat), and in the constant domain CH1 of the second and the third Fab molecule under (ii) the amino acid at position 147 may be substituted by glutamic acid (E) (numbering according to Kabat EU index) and the amino acid at position 213 may be substituted by glutamic acid (E) (numbering according to Kabat EU index).

In some aspects, the first antigen binding moiety of the T cell bispecific antibody (that binds to CD19 and CD3) comprises a heavy chain variable region sequence that is at least about 95%, 96%, 97%, 98%, 99% or 100% identical to the amino acid sequence of SEQ ID NO: 63 or a heavy chain variable region sequence that is at least about 95%, 96%, 97%, 98%, 99% or 100% identical to the amino acid sequence of SEQ ID NO: 66, and a light chain variable region sequence that is at least about 95%, 96%, 97%, 98%, 99% or 100% identical to the amino acid sequence of SEQ ID NO: 35. In some aspects, the first antigen binding moiety comprises the heavy chain variable region sequence of SEQ ID NO: 63 or the heavy chain variable region sequence of SEQ ID NO: 66, and the light chain variable region sequence of SEQ ID NO: 35.

In some aspects, the second and (where present) third antigen binding moiety of the T cell bispecific antibody (that binds to CD19 and CD3) comprise a heavy chain variable region sequence that is at least about 95%, 96%, 97%, 98%, 99% or 100% identical to the amino acid sequence of SEQ ID NO: 73 and a light chain variable region sequence that is at least about 95%, 96%, 97%, 98%, 99% or 100% identical to the amino acid sequence of SEQ ID NO: 74. In some aspects, the second and (where present) third antigen binding moiety comprise the heavy chain variable region of SEQ ID NO: 73 and the light chain variable region of SEQ ID NO: 74.

The Fc domain according to the above aspects may incorporate, singly or in combination, all of the features described hereinabove in relation to Fc domains.

In some aspects, the Fc domain of the T cell bispecific antibody (that binds to CD19 and CD3) comprises a modification promoting the association of the first and the second subunit of the Fc domain, and/or the Fc domain comprises one or more amino acid substitution that reduces binding to an Fc receptor and/or effector function.

In some aspects, the antigen binding moieties and the Fc region are fused to each other by peptide linkers, particularly by peptide linkers as in SEQ ID NO: 75, SEQ ID NO: 76 and SEQ ID NO: 77.

In some aspects, the T cell bispecific antibody (that binds to CD19 and CD3) comprises a polypeptide (particularly two polypeptides) comprising a sequence that is at least 80%, 85%, 90%, 95%, 96%, 97%, 98%, or 99% identical to the sequence of SEQ ID NO: 78, a polypeptide comprising a sequence that is at least 80%, 85%, 90%, 95%, 96%, 97%, 98%, or 99% identical to the sequence of SEQ ID NO: 75, a polypeptide comprising a sequence that is at least 80%, 85%, 90%, 95%, 96%, 97%, 98%, or 99% identical to the sequence of SEQ ID NO: 77, and a polypeptide comprising a sequence that is at least 80%, 85%, 90%, 95%, 96%, 97%, 98%, or 99% identical to the sequence of SEQ ID NO: 79. In some aspects, the T cell bispecific antibody (that binds to CD19 and CD3) comprises a polypeptide (particularly two polypeptides) comprising the sequence of SEQ ID NO: 78, a polypeptide comprising the sequence of SEQ ID NO: 75, a polypeptide comprising the sequence of SEQ ID NO: 77, and a polypeptide comprising the sequence of SEQ ID NO: 79.

In other aspects, the T cell bispecific antibody (that binds to CD19 and CD3) comprises a polypeptide (particularly two polypeptides) comprising a sequence that is at least 80%, 85%, 90%, 95%, 96%, 97%, 98%, or 99% identical to the sequence of SEQ ID NO: 78, a polypeptide comprising a sequence that is at least 80%, 85%, 90%, 95%, 96%, 97%, 98%, or 99% identical to the sequence of SEQ ID NO: 76, a polypeptide comprising a sequence that is at least 80%, 85%, 90%, 95%, 96%, 97%, 98%, or 99% identical to the sequence of SEQ ID NO: 77, and a polypeptide comprising a sequence that is at least 80%, 85%, 90%, 95%, 96%, 97%, 98%, or 99% identical to the sequence of SEQ ID NO: 80. In some aspects, the T cell bispecific antibody (that binds to CD19 and CD3) comprises a polypeptide (particularly two polypeptides) comprising the sequence of SEQ ID NO: 78, a polypeptide comprising the sequence of SEQ ID NO: 76, a polypeptide comprising the sequence of SEQ ID NO: 77, and a polypeptide comprising the sequence of SEQ ID NO: 80.

In some aspects, the disease (to be treated by the T cell bispecific antibody) is cancer.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “cancer” refers to the physiological condition in mammals that is typically characterized by unregulated cell proliferation. Examples of cancer include but are not limited to, carcinoma, lymphoma, blastoma, sarcoma and leukemia. More non-limiting examples of cancers include haematological cancer such as leukemia, bladder cancer, brain cancer, head and neck cancer, pancreatic cancer, biliary cancer, thyroid cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, sarcoma, bone cancer, and kidney cancer. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases.

In some aspects, the cancer is a cancer expressing the target cell antigen of the T cell bispecific antibody.

In some aspects, the cancer is a carcinoembryonic antigen (CEA)-expressing cancer (in particular in aspects, wherein the target cell antigen of the T cell bispecific antibody is CEA). By “CEA-positive cancer” or “CEA-expressing cancer” is meant a cancer characterized by expression or overexpression of CEA on cancer cells. The expression of CEA may be determined for example by an immunohistochemistry (IHC) or flow cytometric assay. In some aspects, the cancer expresses CEA. In some aspects, the cancer expresses CEA in at least 20%, preferably at least 50% or at least 80% of tumor cells as determined by immunohistochemistry (IHC) using an antibody specific for CEA.

In some aspects, the cancer is colon cancer, lung cancer, ovarian cancer, gastric cancer, bladder cancer, pancreatic cancer, endometrial cancer, breast cancer, kidney cancer, esophageal cancer, prostate cancer, or other cancers described herein.

In particular aspects, the cancer is a cancer selected from the group consisting of colorectal cancer, lung cancer, pancreatic cancer, breast cancer, and gastric cancer. In preferred aspects, the cancer is colorectal cancer (CRC). In some aspects, the colorectal cancer is metastatic colorectal cancer (mCRC). In some aspects, the colorectal cancer is microsatellite-stable (MSS) colorectal cancer. In some aspects, the colorectal cancer is microsatellite-stable metastatic colorectal cancer (MSS mCRC).

In some aspects, the cancer is a Wilms tumor protein (WT1)-expressing cancer (in particular in aspects, wherein the target cell antigen of the T cell bispecific antibody is HLA-A2/WT1). By “WT1-positive cancer” or “WT1-expressing cancer” is meant a cancer characterized by expression or overexpression of WT1 in cancer cells. The expression of WT1 may be determined for example by quantitative real-time PCR (measuring WT1 mRNA levels), flow cytometry, immunohistochemistry (IHC) or western blot assays. In some aspects, the cancer expresses WT1. In some aspects, the cancer expresses WT1 in at least 20%, preferably at least 50% or at least 80% of tumor cells as determined by immunohistochemistry (IHC) using an antibody specific for WT1.

In some aspects, the cancer is a haematological cancer. Non-limiting examples of haematological cancers include leukemia (e.g. acute lymphocytic leukemia (ALL), acute myeloid leukemia (AML), chronic lymphcytic leukemia (CLL) chronic myeloid leukemia (CML), hairy cell leukemia (HCL)), lymphoma (e.g. Non-Hodgkin lymphoma (NHL), Hodgkin lymphoma), myeloma (e.g. multiple myeloma (MM)), myelodysplastic syndrome (MDS) and myeloproliferative diseases.

In certain aspects, the cancer is chosen from the group consisting of haematological cancer (such as leukemia), kidney cancer, bladder cancer, skin cancer, lung cancer, colorectal cancer, breast cancer, brain cancer, head and neck cancer and prostate cancer.

In particular aspects, the cancer is a haematological cancer, particularly leukemia, most particularly acute lymphocytic leukemia (ALL) or acute myeloid leukemia (AML). In preferred aspects the cancer is acute myeloid leukemia (AML). In further particular aspects, the cancer is myelodysplastic syndrome (MDS).

In some aspects, the cancer is a CD20-expressing cancer (in particular in aspects, wherein the target cell antigen of the T cell bispecific antibody is CD20). By “CD20-positive cancer” or “CD20-expressing cancer” is meant a cancer characterized by expression or overexpression of CD20 in cancer cells. The expression of CD20 may be determined for example by quantitative real-time PCR (measuring CD20 mRNA levels), flow cytometry, immunohistochemistry (IHC) or western blot assays. In some aspects, the cancer expresses CD20. In some aspects, the cancer expresses CD20 in at least 20%, preferably at least 50% or at least 80% of tumor cells as determined by immunohistochemistry (IHC) using an antibody specific for CD20.

In some aspects, the cancer is a B-cell cancer, particularly a CD20-positive B-cell cancer. In some aspects, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), marginal zone lymphoma (MZL), Multiple myeloma (MM) or Hodgkin lymphoma (HL). In particular aspects, the cancer is selected from the group consisting of Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL) and marginal zone lymphoma (MZL). In more particular aspects, the cancer is NHL, particularly relapsed/refractory (r/r) NHL. In some aspects, the cancer is DLBCL. In some aspects, the cancer is FL. In some aspects, the cancer is MCL. In some aspects, the cancer is MZL.

In some aspects, the cancer is a CD19-expressing cancer (in particular in aspects, wherein the target cell antigen of the T cell bispecific antibody is CD19). By “CD19-positive cancer” or “CD19-expressing cancer” is meant a cancer characterized by expression or overexpression of CD19 in cancer cells. The expression of CD19 may be determined for example by quantitative real-time PCR (measuring CD19 mRNA levels), flow cytometry, immunohistochemistry (IHC) or western blot assays. In some aspects, the cancer expresses CD19. In some aspects, the cancer expresses CD19 in at least 20%, preferably at least 50% or at least 80% of tumor cells as determined by immunohistochemistry (IHC) using an antibody specific for CD19.

In some aspects, the cancer is a B-cell cancer, particularly a CD19-positive B-cell cancer. In some aspects, the cancer is a B-cell lymphoma or a B-cell leukemia. In some aspects, the cancer is non-Hodgkin lymphoma (NHL), acute lymphoblastic leukemia (ALL) or chronic lymphocytic leukemia (CLL).

In some aspects, the cancer is treatable by the T cell bispecific antibody. In some aspects, the T cell bispecific antibody is indicated for the treatment of the cancer.

In some aspects, the cancer is a solid tumor cancer. By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer. Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

In some aspects wherein the target cell antigen of the T cell bispecific antibody is CD19, the disease (to be treated by the T cell bispecific antibody) is an autoimmune disease. In specific aspects, the autoimmune disease is lupus, in particular systemic lupus erythematosus (SLE) or lupus nephritis (LN).

An “individual” or “subject” herein is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). In certain aspects, the individual or subject is a human. In some aspects, the individual has a disease, particularly a disease treatable or to be treated by the T cell bispecific antibody. In some aspects, the individual has cancer, particularly a cancer treatable or to be treated by the T cell bispecific antibody. In particular, an individual herein is any single human subject eligible for treatment who is experiencing or has experienced one or more signs, symptoms, or other indicators of cancer. In some aspects, the individual has cancer or has been diagnosed with cancer, in particular any of the cancers described hereinabove. In some aspects, the individual has locally advanced or metastatic cancer or has been diagnosed with locally advanced or metastatic cancer. The individual may have been previously treated with a T cell bispecific antibody or another drug, or not so treated. In particular aspects, the patient has not been previously treated with T cell bispecific antibody. The patient may have been treated with a therapy comprising one or more drugs other than a T cell bispecific antibody before the T cell bispecific antibody therapy is commenced.

In some aspects, the individual has an elevated serum level of one of more cytokine. In some aspects, said elevated serum level is related to the administration of the T cell bispecific antibody to the individual. Said elevated serum level is in particular as compared to the serum level in a healthy individual, and/or the serum level in an individual (including the same individual) without administration of the T cell bispecific antibody (i.e. in such case the serum level is elevated as compared to the serum level without administration of the T cell bispecific antibody). In some aspects, said one or more cytokine is selected from the group consisting of IL-2, TNF-α, IFN-γ, IL-6 and IL-1β.

A cytokine according to any of the aspects of the invention is preferably a proinflammatory cytokine, in particular one or more cytokine selected from the group consisting of IL-2, TNF-α, IFN-γ, IL-6 and IL-1β. In some aspects, the cytokine is IL-2. In some aspects, the cytokine is TNF-α. In some aspects, the cytokine is IFN-γ. In some aspects, the cytokine is IL-6. In some aspects, the cytokine is IL-1β.

Preferably, a T cell according to any of the aspects of the invention is a cytotoxic T cell. In some aspects the T cell is a CD4⁺ or a CD8⁺ T cell. In some aspects the T cell is a CD4⁺ T cell.

In some aspects, the treatment with or administration of the T cell bispecific antibody may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the treatment with or administration of the T cell bispecific antibody and the TKI may improve the response as compared to treatment with or administration of the T cell bispecific antibody alone (i.e. without the TKI). In some aspects, the treatment or administration of the T cell bispecific antibody and the TKI may increase response rates in a patient population, as compared to a corresponding patient population treated with the T cell bispecific antibody alone (i.e. without the TKI).

The T cell bispecific antibody can be used either alone or together with other agents in a therapy. For instance, a T cell bispecific antibody may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis.

Amino Acid Sequences

| | | SEQ |
|---|---|---|
| | Sequence | ID NO |
| CD3 HCDR1 | GYTMN | 1 |
| CD3 HCDR2 | LINPYKGVSTYNQKFKD | 2 |
| CD3 HCDR3 | SGYYGDSDWYFDV | 3 |
| CD3 LCDR1 | RASQDIRNYLN | 4 |
| CD3 LCDR2 | YTSRLES | 5 |
| CD3 LCDR3 | QQGNTLPWT | 6 |
| CD3 VH | EVQLVESGGGLVQPGGSLRLSCAASGYSFTGYTMNWVRQ | 7 |
| | APGKGLEWVALINPYKGVSTYNQKFKDRFTISVDKSKNTA | |
| | YLQMNSLRAEDTAVYYCARSGYYGDSDWYFDVWGQGTL | |
| | VTVSS | |
| CD3 VL | DIQMTQSPSSLSASVGDRVTITCRASQDIRNYLNWYQQKPG | 8 |
| | KAPKLLIYYTSRLESGVPSRFSGSGSGTDYTLTISSLQPEDFA | |
| | TYYCQQGNTLPWTFGQGTKVEIK | |
| WT1 HCDR1 | SYAIS | 9 |
| WT1 HCDR2 | GIIPIFGTANYAQKFQG | 10 |
| WT1 HCDR3 | SIELWWGGFDY | 11 |
| WT1 LCDR1 | RASQSISSWLA | 12 |
| WT1 LCDR2 | DASSLES | 13 |
| WT1 LCDR3 | QQYEDYTT | 14 |
| WT1 VH | QVQLVQSGAEVKKPGSSVKVSCKASGGTFSSYAISWVRQA | 15 |
| | PGQGLEWMGGIIPIFGTANYAQKFQGRVTITADKSTSTAYM | |
| | ELSSLRSEDTAVYYCARSIELWWGGFDYWGQGTTVTVSS | |
| WT1 VL | DIQMTQSPSTLSASVGDRVTITCRASQSISSWLAWYQQKPG | 16 |
| | KAPKLLIYDASSLESGVPSRFSGSGSGTEFTLTIGSLQPDDFA | |
| | TYYCQQYEDYTTFGQGTKVEIK | |
| WT1 VL- | DIQMTQSPSTLSASVGDRVTITCRASQSISSWLAWYQQKPG | 17 |
| CL(RK) | KAPKLLIYDASSLESGVPSRFSGSGSGTEFTLTIGSLQPDDFA | |
| | TYYCQQYEDYTTFGQGTKVEIKRTVAAPSVFIFPPSDRKLK | |
| | SGTASVVCLLNNFYPREAKVQWKVDNALQSGNSQESVTE | |
| | QDSKDSTYSLSSTLTLSKADYEKHKVYACEVTHQGLSSPVT | |
| | KSFNRGEC | |
| WT1 VH- | QVQLVQSGAEVKKPGSSVKVSCKASGGTFSSYAISWVRQA | 18 |
| CH1(EE)- | PGQGLEWMGGIIPIFGTANYAQKFQGRVTITADKSTSTAYM | |
| Fc(hole, | ELSSLRSEDTAVYYCARSIELWWGGFDYWGQGTTVTVSSA | |
| PGLALA) | STKGPSVFPLAPSSKSTSGGTAALGCLVEDYFPEPVTVSWN | |
| | SGALTSGVHTFPAVLQSSGLYSLSSVVTVPSSSLGTQTYICN | |
| | VNHKPSNTKVDEKVEPKSCDKTHTCPPCPAPEAAGGPSVFL | |
| | FPPKPKDTLMISRTPEVTCVVVDVSHEDPEVKFNWYVDGV | |
| | EVHNAKTKPREEQYNSTYRVVSVLTVLHQDWLNGKEYKC | |
| | KVSNKALGAPIEKTISKAKGQPREPQVCTLPPSRDELTKNQ | |
| | VSLSCAVKGFYPSDIAVEWESNGQPENNYKTTPPVLDSDGS | |
| | FFLVSKLTVDKSRWQQGNVFSCSVMHEALHNHYTQKSLSL | |
| | SP | |
| CD3 VH-CL | EVQLVESGGGLVQPGGSLRLSCAASGYSFTGYTMNWVRQ | 19 |
| | APGKGLEWVALINPYKGVSTYNQKFKDRFTISVDKSKNTA | |
| | YLQMNSLRAEDTAVYYCARSGYYGDSDWYFDVWGQGTL | |
| | VTVSSASVAAPSVFIFPPSDEQLKSGTASVVCLLNNFYPREA | |
| | KVQWKVDNALQSGNSQESVTEQDSKDSTYSLSSTLTLSKA | |
| | DYEKHKVYACEVTHQGLSSPVTKSFNRGEC | |
| WT1 VH- | QVQLVQSGAEVKKPGSSVKVSCKASGGTFSSYAISWVRQA | 20 |
| CH1(EE)-CD3 | PGQGLEWMGGIIPIFGTANYAQKFQGRVTITADKSTSTAYM | |
| VL-CH1- | ELSSLRSEDTAVYYCARSIELWWGGFDYWGQGTTVTVSSA | |
| Fc(knob, | STKGPSVFPLAPSSKSTSGGTAALGCLVEDYFPEPVTVSWN | |
| PGLALA) | SGALTSGVHTFPAVLQSSGLYSLSSVVTVPSSSLGTQTYICN | |
| | VNHKPSNTKVDEKVEPKSCDGGGGSGGGGSDIQMTQSPSS | |
| | LSASVGDRVTITCRASQDIRNYLNWYQQKPGKAPKLLIYYT | |
| | SRLESGVPSRFSGSGSGTDYTLTISSLQPEDFATYYCQQGNT | |
| | LPWTFGQGTKVEIKSSASTKGPSVFPLAPSSKSTSGGTAALG | |
| | CLVKDYFPEPVTVSWNSGALTSGVHTFPAVLQSSGLYSLSS | |
| | VVTVPSSSLGTQTYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSV | |
| | LTVLHQDWLNGKEYKCKVSNKALGAPIEKTISKAKGQPRE | |
| | PQVYTLPPCRDELTKNQVSLWCLVKGFYPSDIAVEWESNG | |
| | QPENNYKTTPPVLDSDGSFFLYSKLTVDKSRWQQGNVFSC | |
| | SVMHEALHNHYTQKSLSLSP | |
| Human WT1 | MGSDVRDLNALLPAVPSLGGGGGCALPVSGAAQWAPVLD | 21 |
| | FAPPGASAYGSLGGPAPPPAPPPPPPPPPHSFIKQEPSWGGA | |
| | EPHEEQCLSAFTVHFSGQFTGTAGACRYGPFGPPPPSQASS | |
| | GQARMFPNAPYLPSCLESQPAIRNQGYSTVTFDGTPSYGHT | |
| | PSHHAAQFPNHSFKHEDPMGQQGSLGEQQYSVPPPVYGCH | |
| | TPTDSCTGSQALLLRTPYSSDNLYQMTSQLECMTWNQMNL | |
| | GATLKGVAAGSSSSVKWTEGQSNHSTGYESDNHTTPILCG | |
| | AQYRIHTHGVFRGIQDVRRVPGVAPTLVRSASETSEKRPFM | |
| | CAYPGCNKRYFKLSHLQMHSRKHTGEKPYQCDFKDCERR | |
| | FSRSDQLKRHQRRHTGVKPFQCKTCQRKFSRSDHLKTHTR | |
| | THTGKTSEKPFSCRWPSCQKKFARSDELVRHHNMHQRNM | |
| | TKLQLAL | |
| VLD peptide | VLDFAPPGA | 22 |
| RMF peptide | RMFPNAPYL | 23 |
| HLA-A2 | GSHSMRYFFTSVSRPGRGEPRFIAVGYVDDTQFVRFDSDAA | 24 |
| | SQRMEPRAPWIEQEGPEYWDGETRKVKAHSQTHRVDLGT | |
| | LRGYYNQSEAGSHTVQRMYGCDVGSDWRFLRGYHQYAY | |
| | DGKDYIALKEDLRSWTAADMAAQTTKHKWEAAHVAEQL | |
| | RAYLEGTCVEWLRRYLENGKETLQRTDAPKTHMTHHAVS | |
| | DHEATLRCWALSFYPAEITLTWQRDGEDQTQDTELVETRP | |
| | AGDGTFQKWAAVVVPSGQEQRYTCHVQHEGLPKPLTLRW | |
| | E | |
| Human CD3 | MQSGTHWRVLGLCLLSVGVWGQDGNEEMGGITQTPYKVS | 25 |
| | ISGTTVILTCPQYPGSEILWQHNDKNIGGDEDDKNIGSDEDH | |
| | LSLKEFSELEQSGYYVCYPRGSKPEDANFYLYLRARVCENC | |
| | MEMDVMSVATIVIVDICITGGLLLLVYYWSKNRKAKAKPV | |
| | TRGAGAGGRQRGQNKERPPPVPNPDYEPIRKGQRDLYSGL | |
| | NQRRI | |
| Cynomolgus | MQSGTRWRVLGLCLLSIGVWGQDGNEEMGSITQTPYQVSI | 26 |
| CD3 | SGTTVILTCSQHLGSEAQWQHNGKNKEDSGDRLFLPEFSE | |
| | MEQSGYYVCYPRGSNPEDASHHLYLKARVCENCMEMDV | |
| | MAVATIVIVDICITLGLLLLVYYWSKNRKAKAKPVTRGAG | |
| | AGGRQRGQNKERPPPVPNPDYEPIRKGQQDLYSGLNQRRI | |
| hIgG1 Fc | DKTHTCPPCPAPELLGGPSVFLFPPKPKDTLMISRTPEVTCV | 27 |
| region | VVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSRDELTKNQVSLTCLVKGFYPSDIAVEWE | |
| | SNGQPENNYKTTPPVLDSDGSFFLYSKLTVDKSRWQQGNV | |
| | FSCSVMHEALHNHYTQKSLSLSP | |
| CD3 HCDR1 | TYAMN | 28 |
| CD3 HCDR2 | RIRSKYNNYATYYADSVKG | 29 |
| CD3 HCDR3 | HGNFGNSYVSWFAY | 30 |
| CD3 LCDR1 | GSSTGAVTTSNYAN | 31 |
| CD3 LCDR2 | GTNKRAP | 32 |
| CD3 LCDR3 | ALWYSNLWV | 33 |
| CD3 VH | EVQLLESGGGLVQPGGSLRLSCAASGFTFSTYAMNWVRQA | 34 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSS | |
| CD3 VL | QAVVTQEPSLTVSPGGTVTLTCGSSTGAVTTSNYANWVQE | 35 |
| | KPGQAFRGLIGGTNKRAPGTPARFSGSLLGGKAALTLSGAQ | |
| | PEDEAEYYCALWYSNLWVFGGGTKLTVL | |
| CEA HCDR1 | EFGMN | 36 |
| CEA HCDR2 | WINTKTGEATYVEEFKG | 37 |
| CEA HCDR3 | WDFAYYVEAMDY | 38 |
| CEA LCDR1 | KASAAVGTYVA | 39 |
| CEA LCDR2 | SASYRKR | 40 |
| CEA LCDR3 | HQYYTYPLFT | 41 |
| CEA VH | QVQLVQSGAEVKKPGASVKVSCKASGYTFTEFGMNWVRQ | 42 |
| | APGQGLEWMGWINTKTGEATYVEEFKGRVTFTTDTSTSTA | |
| | YMELRSLRSDDTAVYYCARWDFAYYVEAMDYVVGQGTTV | |
| | TVSS | |
| CEA VL | DIQMTQSPSSLSASVGDRVTITCKASAAVGTYVAWYQQKP | 43 |
| | GKAPKLLIYSASYRKRGVPSRFSGSGSGTDFTLTISSLQPED | |
| | FATYYCHQYYTYPLFTFGQGTKLEIK | |
| CEA VL-CL | DIQMTQSPSSLSASVGDRVTITCKASAAVGTYVAWYQQKP | 44 |
| | GKAPKLLIYSASYRKRGVPSRFSGSGSGTDFTLTISSLQPED | |
| | FATYYCHQYYTYPLFTFGQGTKLEIKRTVAAPSVFIFPPSDE | |
| | QLKSGTASVVCLLNNFYPREAKVQWKVDNALQSGNSQES | |
| | VTEQDSKDSTYSLSSTLTLSKADYEKHKVYACEVTHQGLS | |
| | SPVTKSFNRGEC | |
| CEA VH- | QVQLVQSGAEVKKPGASVKVSCKASGYTFTEFGMNWVRQ | 45 |
| CH1-Fc(hole, | APGQGLEWMGWINTKTGEATYVEEFKGRVTFTTDTSTSTA | |
| PGLALA) | YMELRSLRSDDTAVYYCARWDFAYYVEAMDYWGQGTTV | |
| | TVSSASTKGPSVFPLAPSSKSTSGGTAALGCLVKDYFPEPVT | |
| | VSWNSGALTSGVHTFPAVLQSSGLYSLSSVVTVPSSSLGTQ | |
| | TYICNVNHKPSNTKVDKKVEPKSCDKTHTCPPCPAPEAAG | |
| | GPSVFLFPPKPKDTLMISRTPEVTCVVVDVSHEDPEVKFNW | |
| | YVDGVEVHNAKTKPREEQYNSTYRVVSVLTVLHQDWLNG | |
| | KEYKCKVSNKALGAPIEKTISKAKGQPREPQVCTLPPSRDE | |
| | LTKNQVSLSCAVKGFYPSDIAVEWESNGQPENNYKTTPPV | |
| | LDSDGSFFLVSKLTVDKSRWQQGNVFSCSVMHEALHNHY | |
| | TQKSLSLSPGK | |
| CD3 VL-CH1 | QAVVTQEPSLTVSPGGTVTLTCGSSTGAVTTSNYANWVQE | 46 |
| | KPGQAFRGLIGGTNKRAPGTPARFSGSLLGGKAALTLSGAQ | |
| | PEDEAEYYCALWYSNLWVFGGGTKLTVLSSASTKGPSVFP | |
| | LAPSSKSTSGGTAALGCLVKDYFPEPVTVSWNSGALTSGV | |
| | HTFPAVLQSSGLYSLSSVVTVPSSSLGTQTYICNVNHKPSNT | |
| | KVDKKVEPKSC | |
| CEA VH- | QVQLVQSGAEVKKPGASVKVSCKASGYTFTEFGMNWVRQ | 47 |
| CH1-CD3 | APGQGLEWMGWINTKTGEATYVEEFKGRVTFTTDTSTSTA | |
| VH-CL- | YMELRSLRSDDTAVYYCARWDFAYYVEAMDYWGQGTTV | |
| Fc(knob, | TVSSASTKGPSVFPLAPSSKSTSGGTAALGCLVKDYFPEPVT | |
| PGLALA) | VSWNSGALTSGVHTFPAVLQSSGLYSLSSVVTVPSSSLGTQ | |
| | TYICNVNHKPSNTKVDKKVEPKSCDGGGGSGGGGSEVQLL | |
| | ESGGGLVQPGGSLRLSCAASGFTFSTYAMNWVRQAPGKGL | |
| | EWVSRIRSKYNNYATYYADSVKGRFTISRDDSKNTLYLQM | |
| | NSLRAEDTAVYYCVRHGNFGNSYVSWFAYWGQGTLVTVS | |
| | SASVAAPSVFIFPPSDEQLKSGTASVVCLLNNFYPREAKVQ | |
| | WKVDNALQSGNSQESVTEQDSKDSTYSLSSTLTLSKADYE | |
| | KHKVYACEVTHQGLSSPVTKSFNRGECDKTHTCPPCPAPE | |
| | AAGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALGAPIEKTISKAKGQPREPQVYTLP | |
| | PCRDELTKNQVSLWCLVKGFYPSDIAVEWESNGQPENNYK | |
| | TTPPVLDSDGSFFLYSKLTVDKSRWQQGNVFSCSVMHEAL | |
| | HNHYTQKSLSLSPGK | |
| CD20 HCDR1 | YSWIN | 48 |
| CD20 HCDR2 | RIFPGDGDTDYNGKFKG | 49 |
| CD20 HCDR3 | NVFDGYWLVY | 50 |
| CD20 LCDR1 | RSSKSLLHSNGITYLY | 51 |
| CD20 LCDR2 | QMSNLVS | 52 |
| CD20 LCDR3 | AQNLELPYT | 53 |
| CD20 VH | QVQLVQSGAEVKKPGSSVKVSCKASGYAFSYSWINWVRQ | 54 |
| | APGQGLEWMGRIFPGDGDTDYNGKFKGRVTITADKSTSTA | |
| | YMELSSLRSEDTAVYYCARNVFDGYWLVYWGQGTLVTVS | |
| | S | |
| CD20 VL | DIVMTQTPLSLPVTPGEPASISCRSSKSLLHSNGITYLYWYL | 55 |
| | QKPGQSPQLLIYQMSNLVSGVPDRFSGSGSGTDFTLKISRVE | |
| | AEDVGVYYCAQNLELPYTFGGGTKVEIK | |
| CD20 VL- | DIVMTQTPLSLPVTPGEPASISCRSSKSLLHSNGITYLYWYL | 56 |
| CL(RK) | QKPGQSPQLLIYQMSNLVSGVPDRFSGSGSGTDFTLKISRVE | |
| | AEDVGVYYCAQNLELPYTFGGGTKVEIKRTVAAPSVFIFPP | |
| | SDRKLKSGTASVVCLLNNFYPREAKVQWKVDNALQSGNS | |
| | QESVTEQDSKDSTYSLSSTLTLSKADYEKHKVYACEVTHQ | |
| | GLSSPVTKSFNRGEC | |
| CD20 VH- | QVQLVQSGAEVKKPGSSVKVSCKASGYAFSYSWINWVRQ | 57 |
| CH1(EE)- | APGQGLEWMGRIFPGDGDTDYNGKFKGRVTITADKSTSTA | |
| Fc(hole, | YMELSSLRSEDTAVYYCARNVFDGYWLVYWGQGTLVTVS | |
| PGLALA) | SASTKGPSVFPLAPSSKSTSGGTAALGCLVEDYFPEPVTVS | |
| | WNSGALTSGVHTFPAVLQSSGLYSLSSVVTVPSSSLGTQTY | |
| | ICNVNHKPSNTKVDEKVEPKSCDKTHTCPPCPAPEAAGGPS | |
| | VFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVCTLPPSRDELT | |
| | KNQVSLSCAVKGFYPSDIAVEWESNGQPENNYKTTPPVLD | |
| | SDGSFFLVSKLTVDKSRWQQGNVFSCSVMHEALHNHYTQ | |
| | KSLSLSP | |
| CD3 VH-CL | EVQLLESGGGLVQPGGSLRLSCAASGFTFSTYAMNWVRQA | 58 |
| | PGKGLEWVSRIRSKYNNYATYYADSVKGRFTISRDDSKNT | |
| | LYLQMNSLRAEDTAVYYCVRHGNFGNSYVSWFAYWGQG | |
| | TLVTVSSASVAAPSVFIFPPSDEQLKSGTASVVCLLNNFYPR | |
| | EAKVQWKVDNALQSGNSQESVTEQDSKDSTYSLSSTLTLS | |
| | KADYEKHKVYACEVTHQGLSSPVTKSFNRGEC | |
| CD20 VH- | QVQLVQSGAEVKKPGSSVKVSCKASGYAFSYSWINWVRQ | 59 |
| CH1(EE)-CD3 | APGQGLEWMGRIFPGDGDTDYNGKFKGRVTITADKSTSTA | |
| VL-CH1- | YMELSSLRSEDTAVYYCARNVFDGYWLVYWGQGTLVTVS | |
| Fc(knob, | SASTKGPSVFPLAPSSKSTSGGTAALGCLVEDYFPEPVTVS | |
| PGLALA) | WNSGALTSGVHTFPAVLQSSGLYSLSSVVTVPSSSLGTQTY | |
| | ICNVNHKPSNTKVDEKVEPKSCDGGGGSGGGGSQAVVTQE | |
| | PSLTVSPGGTVTLTCGSSTGAVTTSNYANWVQEKPGQAFR | |
| | GLIGGTNKRAPGTPARFSGSLLGGKAALTLSGAQPEDEAEY | |
| | YCALWYSNLWVFGGGTKLTVLSSASTKGPSVFPLAPSSKST | |
| | SGGTAALGCLVKDYFPEPVTVSWNSGALTSGVHTFPAVLQ | |
| | SSGLYSLSSVVTVPSSSLGTQTYICNVNHKPSNTKVDKKVE | |
| | PKSCDKTHTCPPCPAPEAAGGPSVFLFPPKPKDTLMISRTPE | |
| | VTCVVVDVSHEDPEVKFNWYVDGVEVHNAKTKPREEQYN | |
| | STYRVVSVLTVLHQDWLNGKEYKCKVSNKALGAPIEKTIS | |
| | KAKGQPREPQVYTLPPCRDELTKNQVSLWCLVKGFYPSDI | |
| | AVEWESNGQPENNYKTTPPVLDSDGSFFLYSKLTVDKSRW | |
| | QQGNVFSCSVMHEALHNHYTQKSLSLSP | |
| Human CD20 | MTTPRNSVNGTFPAEPMKGPIAMQSGPKPLFRRMSSLVGPT | 60 |
| | QSFFMRESKTLGAVQIMNGLFHIALGGLLMIPAGIYAPICVT | |
| | VWYPLWGGIMYIISGSLLAATEKNSRKCLVKGKMIMNSLS | |
| | LFAAISGMILSIMDILNIKISHFLKMESLNFIRAHTPYINIYNC | |
| | EPANPSEKNSPSTQYCYSIQSLFLGILSVMLIFAFFQELVIAGI | |
| | VENEWKRTCSRPKSNIVLLSAEEKKEQTIEIKEEVVGLTETS | |
| | SQPKNEEDIEIIPIQEEEEEETETNFPEPPQDQESSPIENDSSP | |
| CD3 HCDR1 | SYAMN | 61 |
| CD3 HCDR3 | HTTFPSSYVSYYGY | 62 |
| CD3 VH | EVQLLESGGGLVQPGGSLRLSCAASGFQFSSYAMNWVRQA | 63 |
| | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSS | |
| CD3 HCDR1 | SYAMN | 64 |
| CD3 HCDR3 | ASNFPASYVSYFAY | 65 |
| CD3 VH | EVQLLESGGGLVQPGGSLRLSCAASGFTFSSYAMNWVRQA | 66 |
| | PGKGLEWVSRIRSKYNNYATYYADSVKGRFTISRDDSKNT | |
| | LYLQMNSLRAEDTAVYYCVRASNFPASYVSYFAYWGQGT | |
| | LVTVSS | |
| CD19 HCDR1 | DYIMH | 67 |
| CD19 HCDR2 | YINPYNDGSKYTEKFQG | 68 |
| CD19 HCDR3 | GTYYYGPQLFDY | 69 |
| CD19 LCDR1 | KSSQSLETSTGTTYLN | 70 |
| CD19 LCDR2 | RVSKRFS | 71 |
| CD19 LCDR3 | LQLLEDPYT | 72 |
| CD19 VH | QVQLVQSGAEVKKPGASVKVSCKASGYTFTDYIMHWVRQ | 73 |
| | APGQGLEWMGYINPYNDGSKYTEKFQGRVTMTSDTSISTA | |
| | YMELSRLRSDDTAVYYCARGTYYYGPQLFDYWGQGTTVT | |
| | VSS | |
| CD19 VL | DIVMTQTPLSLSVTPGQPASISCKSSQSLETSTGTTYLNWYL | 74 |
| | QKPGQSPQLLIYRVSKRFSGVPDRFSGSGSGTDFTLKISRVE | |
| | AEDVGVYYCLQLLEDPYTFGQGTKLEIK | |
| CD19 VH- | QVQLVQSGAEVKKPGASVKVSCKASGYTFTDYIMHWVRQ | 75 |
| CH1(EE)- | APGQGLEWMGYINPYNDGSKYTEKFQGRVTMTSDTSISTA | |
| CD3 VL-CH1- | YMELSRLRSDDTAVYYCARGTYYYGPQLFDYWGQGTTVT | |
| Fc (knob, | VSSASTKGPSVFPLAPSSKSTSGGTAALGCLVEDYFPEPVTV | |
| PGLALA) | SWNSGALTSGVHTFPAVLQSSGLYSLSSVVTVPSSSLGTQT | |
| | YICNVNHKPSNTKVDEKVEPKSCDGGGGSGGGGSQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALGAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |
| CD19 VH- | QVQLVQSGAEVKKPGASVKVSCKASGYTFTDYIMHWVRQ | 76 |
| CH1(EE)- | APGQGLEWMGYINPYNDGSKYTEKFQGRVTMTSDTSISTA | |
| CD3 VL-CH1- | YMELSRLRSDDTAVYYCARGTYYYGPQLFDYWGQGTTVT | |
| Fc(knob, | VSSASTKGPSVFPLAPSSKSTSGGTAALGCLVEDYFPEPVTV | |
| PGLALA) | SWNSGALTSGVHTFPAVLQSSGLYSLSSVVTVPSSSLGTQT | |
| | YICNVNHKPSNTKVDEKVEPKSCDGGGGSGGGGGQAVVT | |
| | QEPSLTVSPGGTVTLTCGSSTGAVTTSNYANWVQEKPGQA | |
| | FRGLIGGTNKRAPGTPARFSGSLLGGKAALTLSGAQPEDEA | |
| | EYYCALWYSNLWVFGGGTKLTVLSSASTKGPSVFPLAPSS | |
| | KSTSGGTAALGCLVKDYFPEPVTVSWNSGALTSGVHTFPA | |
| | VLQSSGLYSLSSVVTVPSSSLGTQTYICNVNHKPSNTKVDK | |
| | KVEPKSCDKTHTCPPCPAPEAAGGPSVFLFPPKPKDTLMISR | |
| | TPEVTCVVVDVSHEDPEVKFNWYVDGVEVHNAKTKPREE | |
| | QYNSTYRVVSVLTVLHQDWLNGKEYKCKVSNKALGAPIE | |
| | KTISKAKGQPREPQVYTLPPCRDELTKNQVSLWCLVKGFY | |
| | PSDIAVEWESNGQPENNYKTTPPVLDSDGSFFLYSKLTVDK | |
| | SRWQQGNVFSCSVMHEALHNHYTQKSLSLSP | |
| CD19 VH- | QVQLVQSGAEVKKPGASVKVSCKASGYTFTDYIMHWVRQ | 77 |
| CH1(EE)-Fc | APGQGLEWMGYINPYNDGSKYTEKFQGRVTMTSDTSISTA | |
| (hole, | YMELSRLRSDDTAVYYCARGTYYYGPQLFDYWGQGTTVT | |
| PGLALA) | VSSASTKGPSVFPLAPSSKSTSGGTAALGCLVEDYFPEPVTV | |
| | SWNSGALTSGVHTFPAVLQSSGLYSLSSVVTVPSSSLGTQT | |
| | YICNVNHKPSNTKVDEKVEPKSCDKTHTCPPCPAPEAAGGP | |
| | SVFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALGAPIEKTISKAKGQPREPQVCTLPPSRDELT | |
| | KNQVSLSCAVKGFYPSDIAVEWESNGQPENNYKTTPPVLD | |
| | SDGSFFLVSKLTVDKSRWQQGNVFSCSVMHEALHNHYTQ | |
| | KSLSLSP | |
| CD19 VL- | DIVMTQTPLSLSVTPGQPASISCKSSQSLETSTGTTYLNWYL | 78 |
| CL(RK) | QKPGQSPQLLIYRVSKRFSGVPDRFSGSGSGTDFTLKISRVE | |
| | AEDVGVYYCLQLLEDPYTFGQGTKLEIKRTVAAPSVFIFPPS | |
| | DRKLKSGTASVVCLLNNFYPREAKVQWKVDNALQSGNSQ | |
| | ESVTEQDSKDSTYSLSSTLTLSKADYEKHKVYACEVTHQG | |
| | LSSPVTKSFNRGEC | |
| CD3 VH-CL | EVQLLESGGGLVQPGGSLRLSCAASGFQFSSYAMNWVRQA | 79 |
| | PGKGLEWVSRIRSKYNNYATYYADSVKGRFTISRDDSKNT | |
| | LYLQMNSLRAEDTAVYYCVRHTTFPSSYVSYYGYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| CD3 VH-CL | EVQLLESGGGLVQPGGSLRLSCAASGFTFSSYAMNWVRQA | 80 |
| | PGKGLEWVSRIRSKYNNYATYYADSVKGRFTISRDDSKNT | |
| | LYLQMNSLRAEDTAVYYCVRASNFPASYVSYFAYWGQGT | |
| | LVTVSSASVAAPSVFIFPPSDEQLKSGTASVVCLLNNFYPRE | |
| | AKVQWKVDNALQSGNSQESVTEQDSKDSTYSLSSTLTLSK | |
| | ADYEKHKVYACEVTHQGLSSPVTKSFNRGEC | |
| Human CD19 | MPPPRLLFFLLFLTPMEVRPEEPLVVKVEEGDNAVLQCLKG | 81 |
| | TSDGPTQQLTWSRESPLKPFLKLSLGLPGLGIHMRPLAIWLF | |
| | IFNVSQQMGGFYLCQPGPPSEKAWQPGWTVNVEGSGELFR | |
| | WNVSDLGGLGCGLKNRSSEGPSSPSGKLMSPKLYVWAKD | |
| | RPEIWEGEPPCLPPRDSLNQSLSQDLTMAPGSTLWLSCGVP | |
| | PDSVSRGPLSWTHVHPKGPKSLLSLELKDDRPARDMWVM | |
| | ETGLLLPRATAQDAGKYYCHRGNLTMSFHLEITARPVLWH | |
| | WLLRTGGWKVSAVTLAYLIFCLCSLVGILHLQRALVLRRK | |
| | RKRMTDPTRRFFKVTPPPGSGPQNQYGNVLSLPTPTSGLGR | |
| | AQRWAAGLGGTAPSYGNPSSDVQADGALGSRSPPGVGPEE | |
| | EEGEGYEEPDSEEDSEFYENDSNLGQDQLSQDGSGYENPED | |
| | EPLGPEDEDSFSNAESYENEDEELTQPVARTMDFLSPHGSA | |
| | WDPSREATSLGSQSYEDMRGILYAAPQLRSIRGQPGPNHEE | |
| | DADSYENMDNPDGPDPAWGGGGRMGTWSTR | |

### BRIEF DESCRIPTION OF THE DRAWINGS

FIG. 1. Real time killing of red fluorescent A375 cells loaded with RMF peptides by 10 nM HLA-A2 WT-1-TCB (A) and of red fluorescent MKN45 cells by 1 nM CEA-TCB (B) in the presence of different dasatinib concentrations ranging from 100 nM to 0 nM. (A) A375 NucLight Red (NLR) target cells were pulsed with RMF peptides (2 hours before killing assay) and co-cultured with HLA-A2 WT1-TCB, dasatinib (dasa) and PBMCs, effector to target ratio (E:T)=10:1 (E:T=50 000 PBMCs:5000 target cells). Killing was followed by Incucyte® (1 scan every 3 hours, zoom 10×, phase and red 400 ms acquisition time). % Killing was measured by normalizing total red area with values at t=0 hour and target cells+PBMCs+dasatinib (without TCB) control wells for each time point. Means of technical replicates+/−SEM for 1 donor. (B) MKN45 NLR target cells were co-cultured with CEA TCB, dasatinib and PBMCs, E:T=10:1 (E:T=50 000 PBMCs:5000 target cells). Killing was followed by Incucyte (1 scan every 3 hours, zoom 10×, phase and red 400 ms acquisition time). % Killing was measured by normalizing total red area with values at t=0 hour and target cells+PBMCs+dasatinib control wells for each time point. Means of technical replicates+/−SEM for 1 donor.

FIG. 2. Cytokine release. Supernatant were collected at the endpoint (96 hours) of the assay in FIG. 1 and cytokines ((A) IFNγ, (B) IL-2, (C) TNF-α) measured by multiplex cytokine analysis (Luminex). 1 nM CEA-TCB, 1 donor.

FIG. 3. In vitro killing assay set-up and timelines. PBMCs were co-cultured with SKM-1 target cells (E:T=10:1) and 10 nM HLA-A2 WT-1-TCB. Dasatinib (100 nM) was added after 24 hours of activation. PBMCs were labelled with CellTrace™ Violet (CTV) to allow T cell proliferation assessment.

FIG. 4. T cell activation. CD25 and CD69 expression on CD4+ and CD8+ T cells was measured by FACS after 24h and 48h of activation in the presence and absence of dasatinib, according to the assay of FIG. 3. (A) CD25 expression on CD8+ T cells, (B) CD25 expression on CD4+ T-cells, (C) CD69 expression on CD8+ T cells, (D) CD69 expression on CD4+ T cells.

FIG. 5. Cytokine release. Supernatants were collected from the assay of FIG. 3 at 24h and 48h, and cytokines (IFN-γ (A), TNF-α (B) and IL-2 (C)) measured using a multiplex kit (Luminex). Mean+/−SD of 3 donors.

FIG. 6. T cell proliferation. In the assay of FIG. 3, proliferation of CD4+ (A) and CD8+ (B) T cells was measured by FACS 144h after stimulation with HLA-A2 WT-1 TCB by analysis of CTV dye dilution.

FIG. 7. Counts of CD4+ and CD8+ T cells. In the assay of FIG. 3, CD4+ (A) and CD8+(B) T cells counts were measured by FACS, mean of 3 donors+/−SD. paired t test, one tailed p value: 0.1234 (ns), 0.0332 (*), 0.0021 (**), 0.0002 (***), <0.0001 (****).

FIG. 8. In vitro killing assay set-up and timelines. PBMCs were co-cultured with carboxyfluorescein succinimidyl ester (CFSE) labelled SKM-1 target cells (E:T=5:1) and 10 nM HLA-A2 WT-1-TCB for 20h. Activated PBMCs were washed and restimulated on fresh, CTV labelled SKM-1 target cells (E:T=5:1) and 10 nM HLA-A2 WT-1-TCB in the presence or absence of 100 nM dasatinib.

FIG. 9. T cell activation at 20 hours, after stimulation with HLA-A2 WT-1 TCB, and before restimulation with HLA-A2 WT-1-TCB with and without dasatinib, according to the assay of FIG. 8. CD69 and CD25 expression on CD4+ and CD8+ T cell was measured by FACS. Data are shown as mean of 3 donors+/−SEM. (A) CD69 expression on CD8+ T cells, (B) CD25 expression on CD8+ T cells, (C) CD69 expression on CD4+ T cells, (D) CD25 expression on CD4+ T-cells.

FIG. 10. SKM-1 target cell viability upon first stimulation with 10 nM HLA-A2 WT-1-TCB in the absence of dasatinib (left) and upon second stimulation with 10 nM HLA-A2 WT-1-TCB in the presence of 100 nM dasatinib (right). Target cell viability was measured by FACS after 44h in the assay of FIG. 8. Dead CFSE labelled SKM-1 cells were gated on positive near infrared (NIR) cells (left side). Dead CTV labelled SKM-1 cells were gated on positive NIR cells (right side).

FIG. 11. HLA-A2 WT-1-TCB dose response for dead CFSE and CTV SKM-1 cells used for first and second stimulation in the presence and absence of dasatinib in the assay of FIG. 8. Mean+/−SEM of 3 donors, two-tailed paired t test (for each concentration), 2 tailed p values: 0.1234 (ns), 0.0332 (*), 0.0021 (**), 0.0002 (***), <0.0001 (****).

FIG. 12. Cytokine release after the second stimulation with HLA-A2 WT-1-TCB with and without dasatinib. Supernatants from the killing assay of FIG. 8 were collected 24 hours after the second stimulation and IFN-γ (A), TNF-α (B) and IL-2 (C) were measured with a multiplex cytokine kit (Luminex technology). n=3 donors.

FIG. 13. In vitro killing assay set-up and timelines to assess T cell degranulation. PBMCs were co-cultured with SKM-1 target cells (E:T=5:1) and 10 nM HLA-A2 WT-1-TCB in the presence and absence of 100 nM dasatinib. Golgistop and Golgiplug (both BD) and CD107a antibody were added 3 hours after activation with TCB to prevent cytokine externalization.

FIG. 14. CD107a+ populations among CD4+ and CD8+ T cells in the presence and absence of dasatinib for one representative donor, in the assay of FIG. 13. Expression of CD107a on CD4+ and CD8+ T cells was measured by FACS at 24h.

FIG. 15. Percentages of CD107a+ cells among CD4+ (A) and CD8+ (B) T cells in the presence and absence of dasatinib in the assay of FIG. 13. Expression of CD107a on CD4+ and CD8+ T cells was measured by FACS at 24h. Mean of 3 donors+/−SD.

FIG. 16. In vitro killing assay set-up and timelines. PBMCs were stimulated on MKN45 NLR (E:T=10:1) target cells with 1 nM CEA-TCB for 96 hours. Activated PBMCs were washed and restimulated on new MKN45 NLR target cells (E:T=10:1) with 1 nM CEA-TCB in the presence of 25 nM dasatinib for 72 hours. Activated PBMCs were washed to remove dasatinib and restimulated on new MKN45 NLR target cells (E:T=10:1) with 1 nM CEA-TCB (“ON/OFF/ON”, see upper row of table), or vice versa (“OFF/ON/OFF”, see lower row of table). Killing was followed by Incucyte®.

FIG. 17. Real time killing of MKN45 NLR target cells by CEA-TCB in the presence of 25 nM dasatinib (0-72 h) upon first stimulation and in the absence of 25 nM dasatinib (96-170 h) upon restimulation, in the assay of FIG. 16 (“OFF/ON”).

FIG. 18. Cytokine levels in the supernatants of the assay after first stimulation in the presence of 25 nM dasatinib and after second stimulation in the absence of dasatinib, in the assay of FIG. 16 (“OFF/ON”). Cytokines ((A) IFN-γ, (B) IL-2, (C) TNF-α) were measured with a multiplex cytokine kit (Luminex technology). 1 donor.

FIG. 19. Real time killing of MKN45 NLR target cells by CEA-TCB in the assay of FIG. 16 (“ON/OFF/ON”).

FIG. 20. Cytokine levels in the supernatants of the assay after first stimulation in the absence of 25 nM dasatinib and after second stimulation in the presence of 25 nM dasatinib, in the assay of FIG. 16 (“ON/OFF”). Cytokines ((A) IFN-γ, (B) IL-2, (C) TNF-α) were measured with a multiplex cytokine kit (Luminex technology). 1 donor.

FIG. 21. CEA-TCB mediated target cell killing. (A) PBMCs were stimulated on MKN45 NLR (E:T=10:1) target cells with 1 nM CEA-TCB for 96 hours in the presence and absence of dasatinib. (B) Activated PBMCs were washed to remove dasatinib and restimulated on new MKN45 NLR target cells (E:T=10:1) with 1 nM CEA-TCB. Killing was followed by Incucyte®.

FIG. 22. Cytokine release. Supernatants were collected after first and second stimulation in the assay of FIG. 16 and cytokines ((A) TNF-α, (B) IFN-γ, (C) IL-2) measured using a multiplex cytokine kit (Luminex).

FIG. 23. In vitro killing assay set-up and timelines. PBMCs were co-cultured with carboxyfluorescein succinimidyl ester (CFSE) labelled Z138 target cells (E:T=5:1) and 10 nM 1 nM CD20-TCB for 20h. Activated PBMCs were washed and restimulated on fresh, CTV labelled Z138 target cells (E:T=5:1) and 1 nM CD20-TCB in the presence or absence of 100 nM dasatinib.

FIG. 24. Dead Z138 cells upon first and second stimulation with 1 nM CD20-TCB in the presence and absence of 100 nM dasatinib in the assay of FIG. 23, as measured by flow cytometry. Cells were collected 20h after the first and 24 h after the second stimulation and stained with live dead NIR dye. Mean of technical replicates+/−SD. N=3 donors.

FIG. 25. Dead Z138 cells upon second stimulation with 1 nM CD20-TCB in the presence and absence of 100 nM dasatinib in the assay of FIG. 23, as measured by flow cytometry. Cells were collected 24 h after second stimulation and stained with live dead NIR dye. Mean of technical replicates+/−SD. Three donors D1-D3 (A-C).

FIG. 26. In vitro killing assay set-up. PBMCs were co-cultured with CellTrace™ Violet (CTV)-labelled SUDLH-8 tumor cells (E:T=10:1) and escalating concentrations of CD19-TCB in the presence and absence of 100 nM dasatinib.

FIG. 27. 100 nM dasatinib prevents killing of CTV labelled SUDLH-8 cells by CD19-TCB. The killing of SUDLH-8 tumor cells was measured by flow cytometry using a Live Dead Near InfraRed (NIR) dye allowing for exclusion of dead cells (24 hrs) in the assay described in FIG. 26. Mean of n=3 donors+SD. One way ANOVA, Friedman test, p-value: 0.1234 (ns), 0.0332 (*), 0.0021 (**), 0.0001 (***), <0.0001 (****).

FIG. 28. 100 nM dasatinib prevents CD19-TCB-induced CD4+ T cell activation. The expression of CD69 (A) and CD25 (B) on CD4+ T cells was measured by flow cytometry at 24 hrs in the assay described in FIG. 26. Mean of n=3 donors+SD. One way ANOVA, Friedman test, p-value: 0.1234 (ns), 0.0332 (*), 0.0021 (**), 0.0001 (***), <0.0001 (****).

FIG. 29. 100 nM dasatinib prevents CD19-TCB-induced CD8+ T cell activation. The expression of CD69 (A) and CD25 (B) on CD8+ T cells was measured by flow cytometry at 24 hrs in the assay described in FIG. 26. Mean of n=3 donors+SD. One way ANOVA, Friedman test, p-value: 0.1234 (ns), 0.0332 (*), 0.0021 (**), 0.0001 (***), <0.0001 (****).

FIG. 30. 100 nM dasatinib prevents CD19-TCB-induced cytokine release. The levels of IL-2 (A), IFN-γ (B), TNF-α (C), IL-6 (D), GM-CSF (E) and IL-8 (F) were measured by Luminex in the supernatant of the assay described in FIG. 26. 1 representative donor out of 3.

FIG. 31. 100 nM dasatinib prevents cytokine release induced by 1 nM CD19-TCB. The levels of IFN-γ (A), TNF-α (B), IL-2 (C), IL-6 (D), GM-CSF (E) and IL-8 (F) were measured by Luminex in the supernatant of the assay described in FIG. 26. Mean of n=3 donors+/−SEM.

FIG. 32. Timelines and dosing schedule of in vivo experiment assessing effect of dasatinib (50 mg/kg) on CD19-TCB induced cytokine release and B cell depletion in humanized NSG mice. Humanized NSG mice were co-treated with 0.5 mg/kg CD19-TCB (i.v.) and 50 mg/kg dasatinib (p.o.) twice per day. Blood was collected by tail vein bleeding at 1.5 hrs, 6 hrs and 48 hrs after treatment with CD19-TCB. At 72 hrs, blood was collected retro-orbitally, before the termination of the experiment. N=4 mice per group.

FIG. 33. Dasatinib prevents CD19-TCB dependent B cell depletion in the blood of mice from the experiment described in FIG. 32. Representative flow cytometry dot plots of CD20+ B cells gated among human CD45+ cells in the blood (48 hrs) of animals treated with vehicle (A), 0.5 mg/kg CD19-TCB (B), or 0.5 mg/kg CD19-TCB and 50 mg/kg dasatinib (C).

FIG. 34. Dasatinib prevents CD19-TCB dependent B cell depletion until 48 hrs post treatment in vivo. CD20+ B cell count was measured by flow cytometry in the blood of the animals from the experiment described in FIG. 32 at 48 hrs (A) and 72 hrs (B). Mean of n=4 mice+/−SEM. One way ANOVA, Kruskal Wallis test, p-value: 0.1234 (ns), 0.0332 (*), 0.0021 (**), 0.0001 (***), <0.0001 (****).

FIG. 35. Dasatinib prevents CD19-TCB induced cytokine release in vivo. Serum was collected from blood samples collected 1.5 hrs post treatment with CD19-TCB in the experiment described in FIG. 32. The levels of IL-2 (A), TNF-α (B), IFN-γ (C) and IL-6 (D) are measured by Luminex. Mean of n=4 mice+/−SEM. One way ANOVA, Kruskal Wallis test, p-value: 0.1234 (ns), 0.0332 (*), 0.0021 (**), 0.0001 (***), <0.0001 (****).

FIG. 36. Dasatinib prevents CD19-TCB induced cytokine release in vivo. Serum is collected from blood samples collected 6 hrs post treatment with CD19-TCB in the experiment described in FIG. 32. The levels of IL-2 (A), TNF-α (B), IFN-γ (C) and IL-6 (D) are measured by Luminex. Mean of n=4 mice+/−SEM. One way ANOVA, Kruskal Wallis test, p-value: 0.1234 (ns), 0.0332 (*), 0.0021 (**), 0.0001 (***), <0.0001 (****).

FIG. 37. Humanized NSG mice were engrafted with a lymphoma PDX (5 million cells, s.c.). When tumors reached 200 mm³, mice were randomized in groups of 8 or 7 based on their tumor size. They were treated with vehicle (i.v.), 0.5 mg/kg CD19-TCB (i.v.) as a monotherapy, 20 mg/kg dasatinib (p.o.) alone or in combination with 0.5 mg/kg CD19-TCB (i.v.). The serum of each mouse in the vehicle, CD19-TCB, and CD19-TCB+dasatinib groups and of n=4 mice in the dasatinib group was collected by tail-vein bleeding 6 hrs after the first treatment with CD19-TCB.

FIG. 38. Cytokine levels in each individual mouse from the experiment described in FIG. 37. The levels of IFN-γ (A), TNF-α (B), IL-2 (C) and IL-6 (D) were measured in the serum by multiplex cytokine analysis using Luminex. Mean of n=6-8 mice+/−SEM with *p≤0.05, **p≤0.01, ***p≤0.001 by 1 way ANOVA (Kruskal Wallis test).

FIG. 39. Body weight loss for each individual mouse from the experiment described in FIG. 37. The change in body weight [%] is measured as a percentage of the body weight before first treatment with CD19-TCB for each mouse. Box and whiskers showing minimum to maximum values of n=6-8 mice per group.

FIG. 40. Tumor growth curves of the experiment described in FIG. 37. Tumor growth curves were plotted from tumor volumes measured using a Caliper, mean of n=6-8 mice+SD with **p≤0.01 by 1 way ANOVA (Kruskal Wallis test).

### EXAMPLES

The following are examples of methods and compositions of the invention. It is understood that various other aspects may be practiced, given the general description provided above.

### Example 1. Dasatinib is a Potent Inhibitor of TCB-Mediated Target Cell Killing at Pharmacologically Relevant Dose

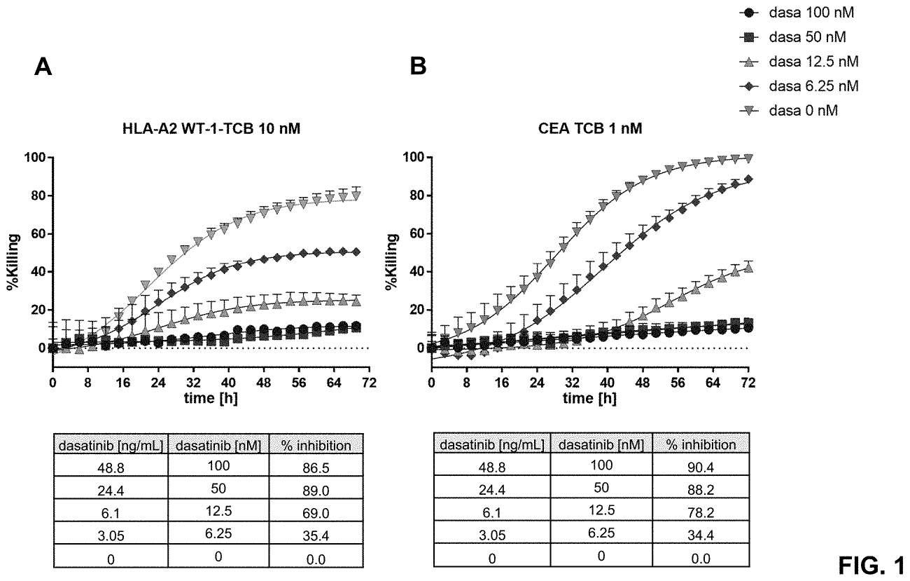

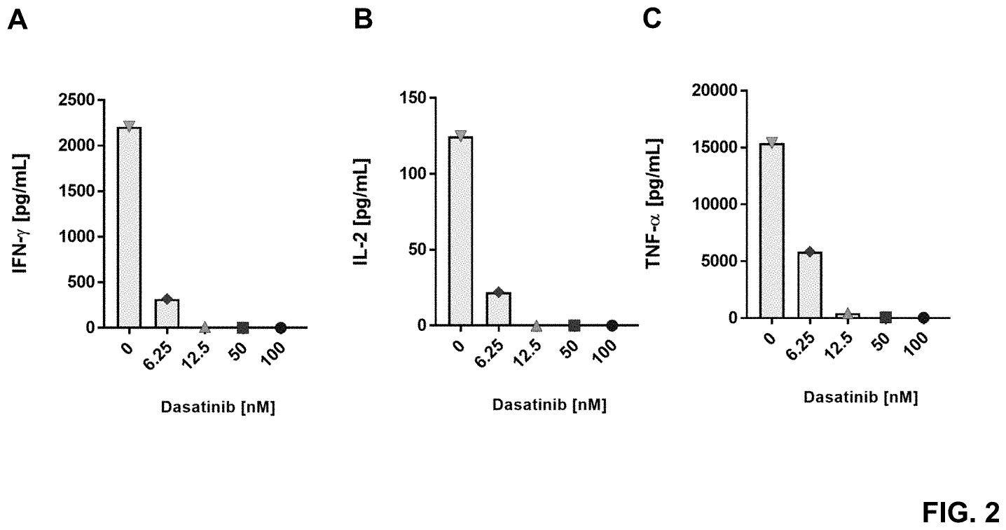

To assess the inhibitory effect of dasatinib on TCB-mediated target-cell killing, we conducted killing assays using peripheral blood mononuclear cells (PBMCs), NucLight Red (NLR) target-cells and respective TCB in media supplemented with escalating concentrations of dasatinib. The Incucyte® system (Essen Bioscience) was used to capture the loss of red fluorescent protein signal over time as a readout of target-cell killing. A concentration of 100 nM (48.8 ng/mL) and 50 nM (24.4 ng/mL) dasatinib resulted in 90.4% and 88.2% inhibition of target-cell killing, respectively, for 1 nM CEA-TCB (SEQ ID NOs 28-47) and 86.5% and 89.0% inhibition of target-cell killing, respectively, for 10 nM HLA-A2 WT1-TCB (SEQ ID NOs 1-20) (FIG. 1). A concentration of 12.5 nM dasatinib resulted in 69% inhibition of target-cell killing for 1 nM CEA-TCB and 78.2% inhibition of target-cell killing for 10 nM HLA-A2 WT-1-TCB. For concentrations below 12.5 nM, dasatinib combined with 1 nM CEA-TCB and 10 nM HLA-A2 WT-1-TCB only partially inhibited killing (FIG. 1). Moreover, at 1 nM CEA-TCB, treatment with a concentration of dasatinib above 12.5 nM prevented the release of IFN-γ, IL-2 and TNF-α as opposed to a lower concentrations of dasatinib and the positive control where dasatinib was not added (FIG. 2). Overall, this data suggest that dasatinib can efficiently prevent T cell mediated target-cell lysis triggered by PBMCs stimulated with both TCB above the threshold in vitro concentration of 12.5 nM.

We then verified if the in vitro dose of dasatinib resulting in inhibition of target-cell killing would translate into one of the pharmacologically active doses as obtained using the approved dose schedule for dasatinib. Therefore, we compared the in vitro dose to the PK parameters Cₘᵢₙ, Cₘₐₓ and steady state concentrations in patients exposed to the different label pharmacological doses for dasatinib. Wang et al. reported that the PK parameters derived from 146 patients treated with 100 mg dasatinib QD is associated with the Cₘᵢₙ value of 2.61 ng/mL and Cₘₐₓ value of 54.6 ng/mL (Wang et al., Clinical Pharmacology: advances and applications (2013) 5, 85-97). Hence, the in vitro dose of 12.5 nM (6 ng/mL) appears translatable into the dasatinib dosing regimen of 100 mg once daily (QD) in patients so that dasatinib is effective at pharmacological label dose to prevent undesired TCB-mediated target-cell killing.

### Example 2. Dasatinib Rapidly Switches Off TCB-Induced T Cell Functionality

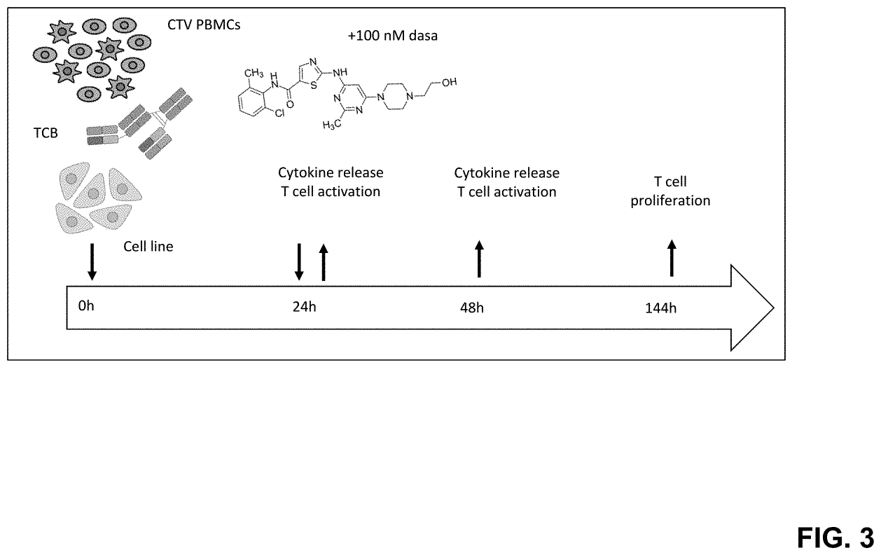

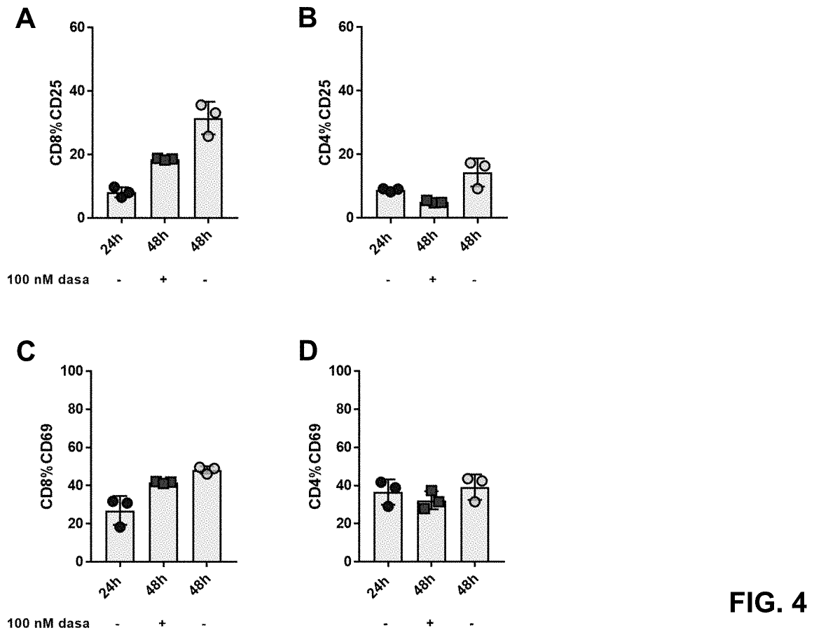

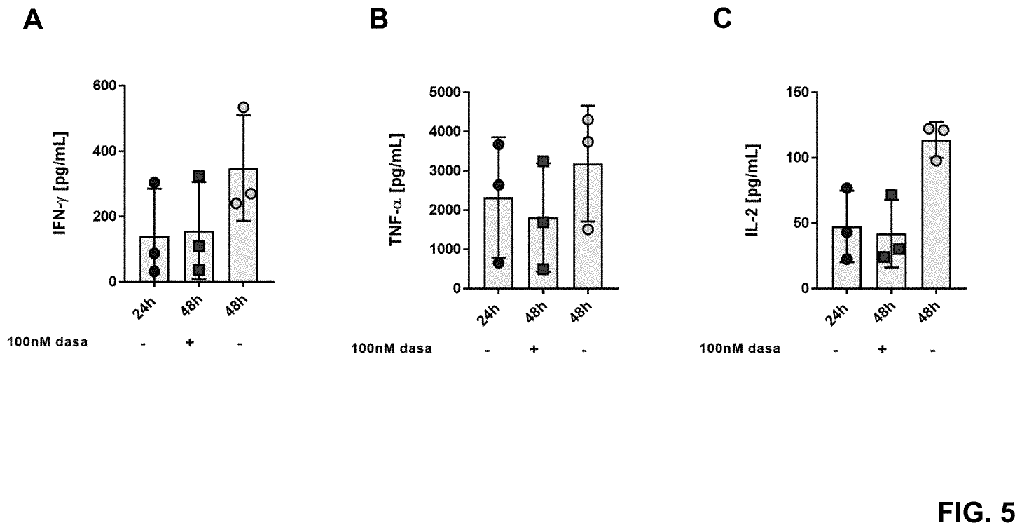

To evaluate if dasatinib could act as a rapid and potent inhibitor of activated T cells, we first stimulated PBMCs on SKM-1 tumor cells with HLA-A2 WT-1-TCB for 24 hours. We then supplemented these activated effector cells with 100 nM dasatinib (FIG. 3). Expression of CD69 and CD25 on CD8+ and CD4+ T cells at 24 hours showed a partially activated phenotype for T cells stimulated with 10 nM HLA-A2 WT-1 TCB (FIG. 4). IFN-γ, TNF-α and IL-2 were also found in the supernatants of these killing assays after 24 hours of activation with 10 nM HLA-A2 WT-1-TCB, revealing T cell activation (FIG. 5).

Upon addition of 100 nM dasatinib at 24h, expression of early activation marker CD69 and late activation marker CD25 on CD4+ and CD8+ T cells at 48 hours was found at an intermediate level between expression measured at 24 hours and expression measured at 48 hours in samples with no dasatinib treatment (FIG. 4). CD25 and CD69 expression on CD4+ and CD8+ T cells measured at 48 hours in comparison to samples not treated with dasatinib highlight that the addition of 100 nM dasatinib rapidly blocked the expression of phenotypic activation markers.

We also looked at the cytokine levels found in the supernatants of killing assays at 48 hours to assess the impact of dasatinib on T cell-mediated cytokine release. Interestingly, no difference was observed for IFN-γ, TNF-α and IL-2 levels measured at 24 hours (0 nM dasatinib) and 48 hours (100 nM dasatinib), as opposed to the positive controls that did not receive dasatinib at 48 hours (FIG. 5). This indicates that the addition of 100 nM dasatinib at 24 hours rapidly prevented the release of cytokines from activated T cells.

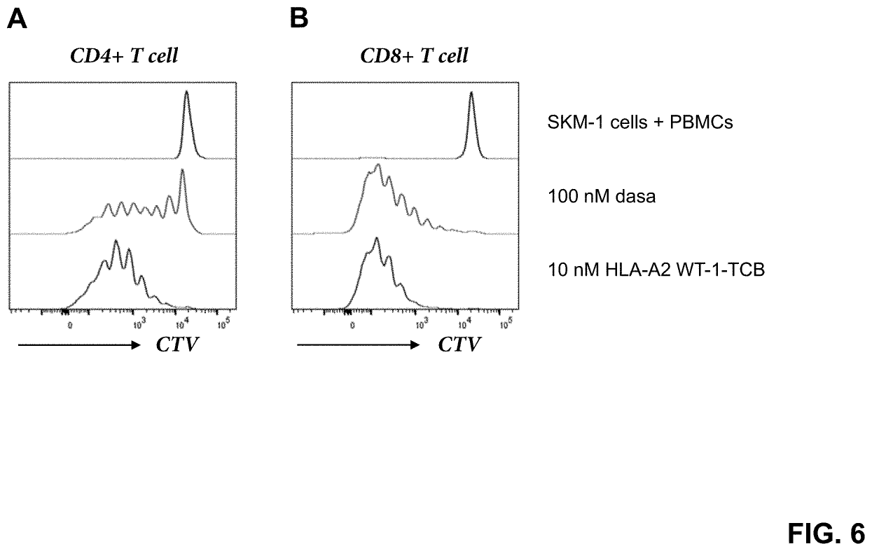

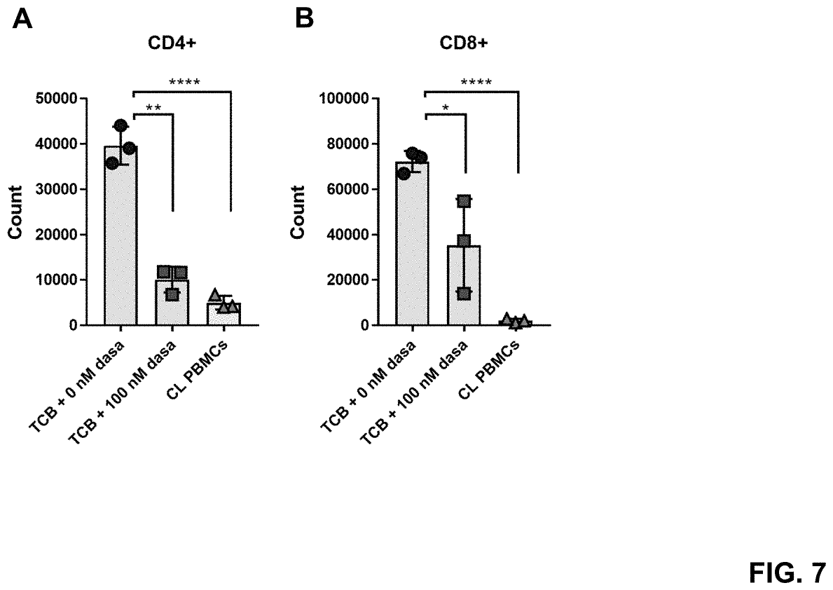

Furthermore, we assessed T cell proliferation 120 hours after the addition of 100 nM dasatinib in the killing assay by measuring the dilution peaks of the CellTrace™ violet (CTV) dye by flow cytometry. As shown in FIG. 6, treatment with 10 nM HLA-A2 WT-1-TCB and 100 nM dasatinib delayed the proliferation peaks of CD4+ and CD8+ with a stronger effect on CD4+ T cells, in comparison to the positive control, which was only treated with 10 nM HLA-A2 WT-1-TCB. The addition of dasatinib to the system at 24 hours resulted in the partial proliferation of T cells when compared to the negative control SKM-1 target cells and PBMCs (upper trace in FIG. 6) where no proliferation peaks were observed. These early proliferation peaks were induced over the first 24 hours of activation in absence of 100 nM dasatinib. Additionally, CD4+ and CD8+ T cell counts were significantly higher in positive control samples which were not treated with 100 nM dasatinib, than in samples treated with 100 nM dasatinib (FIG. 7). The CD8+ T cell count was higher in samples treated with 100 nM dasatinib than in the SKM-1 cells and PBMCs negative control sample (FIG. 7B). The CD4+ T cell count was not higher in samples treated with 100 nM dasatinib than in the SKM-1 cells and PBMCs negative control samples (FIG. 7A). Overall, this indicates that 100 nM dasatinib inhibited TCB-induced T cell proliferation with a stronger impact on CD4+ T cells than CD8+ T cells.

Altogether, dasatinib treatment rapidly resulted in the downregulation of T cell activation, cytokine release and proliferation suggesting that it induced a loss of T cell functionality. However, this assay did not allow the evaluation of the effects of dasatinib on TCB mediated target cell killing since most of the SKM-1 tumors cell were dead after 24 h and prior to the addition of dasatinib.

### Example 3. Dasatinib Prevents TCB-Induced Cytotoxicity of Activated T Cells

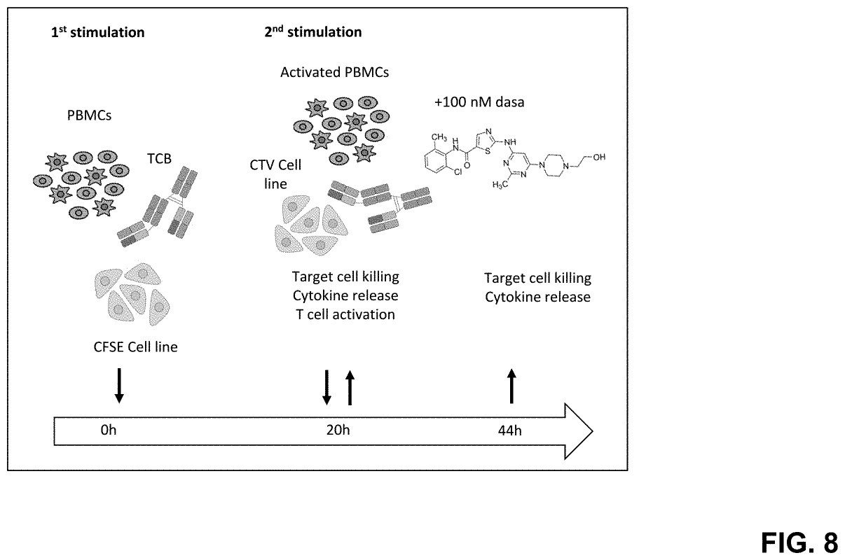

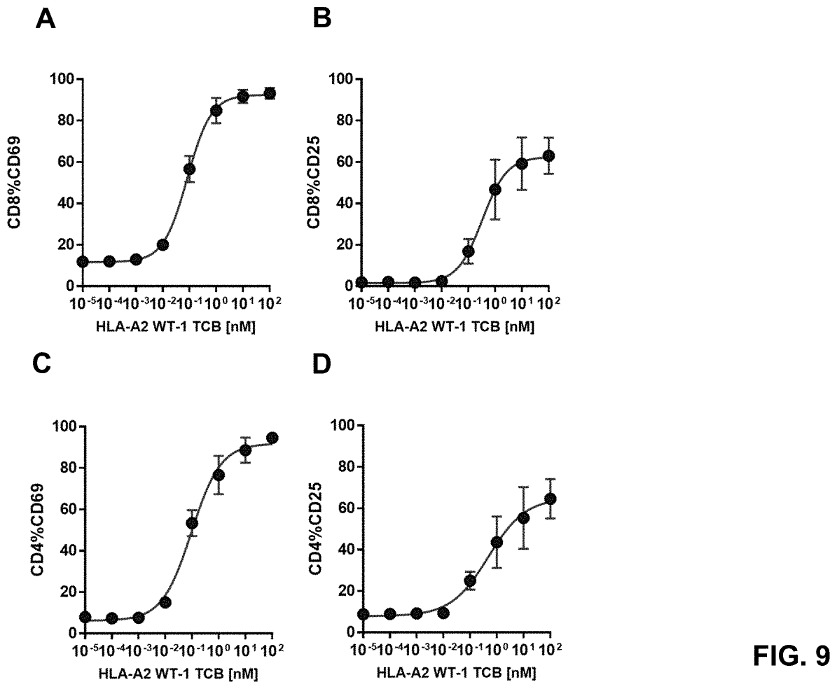

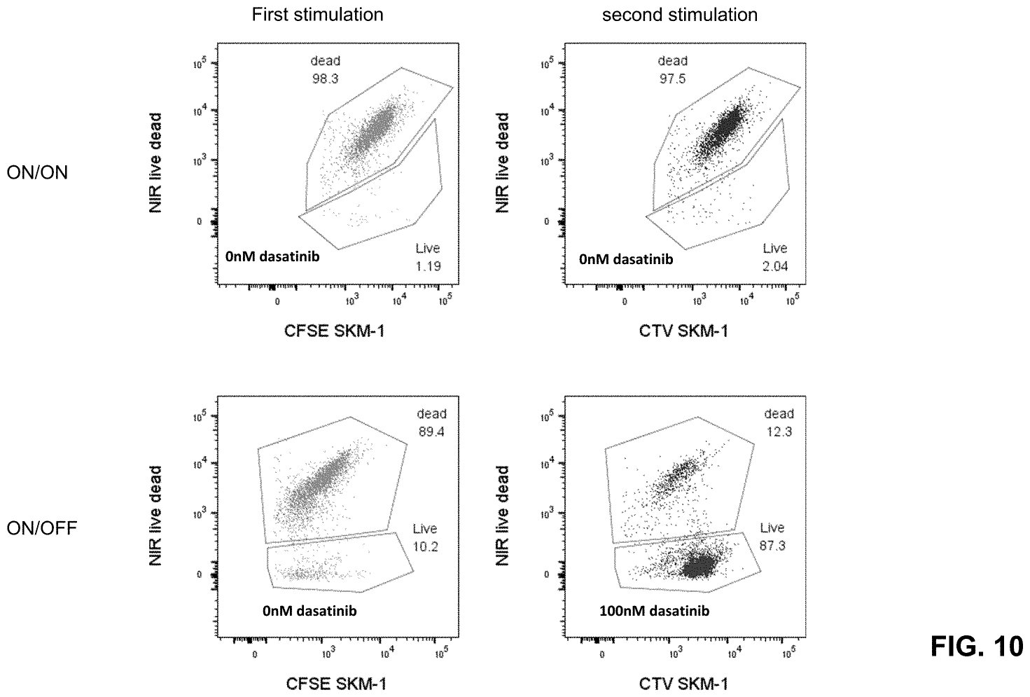

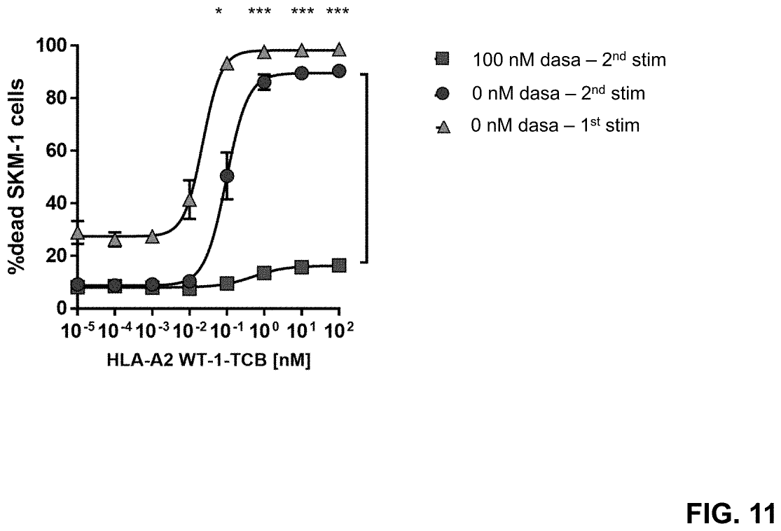

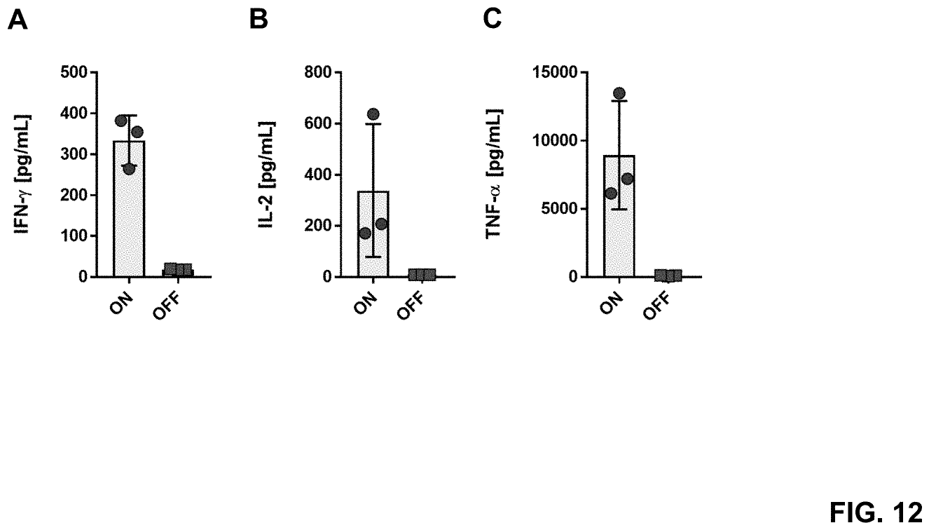

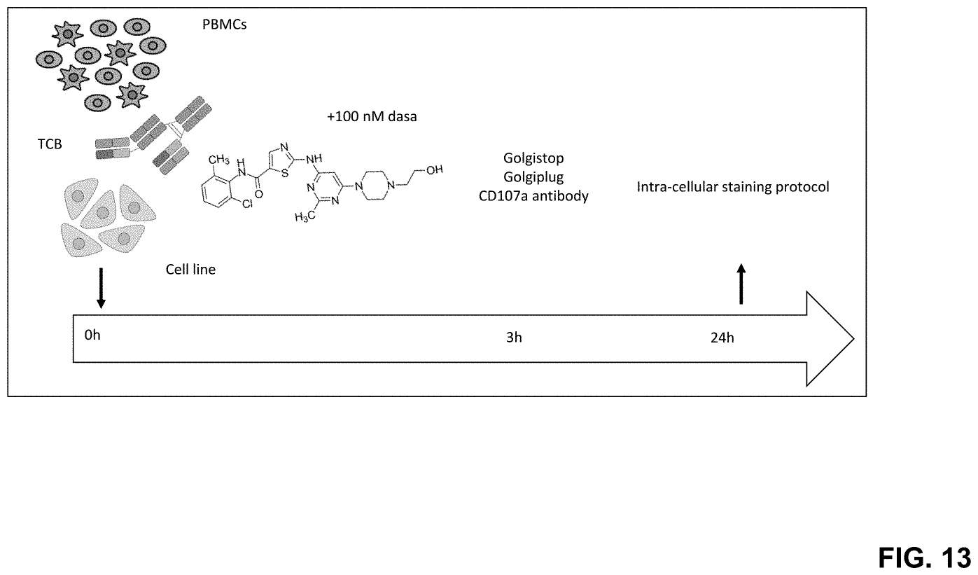

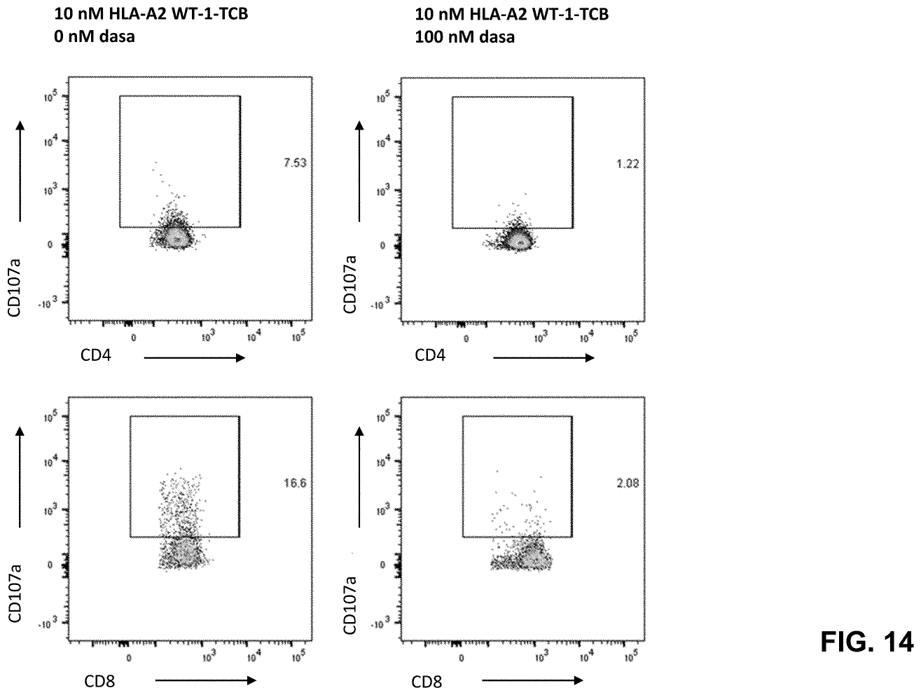

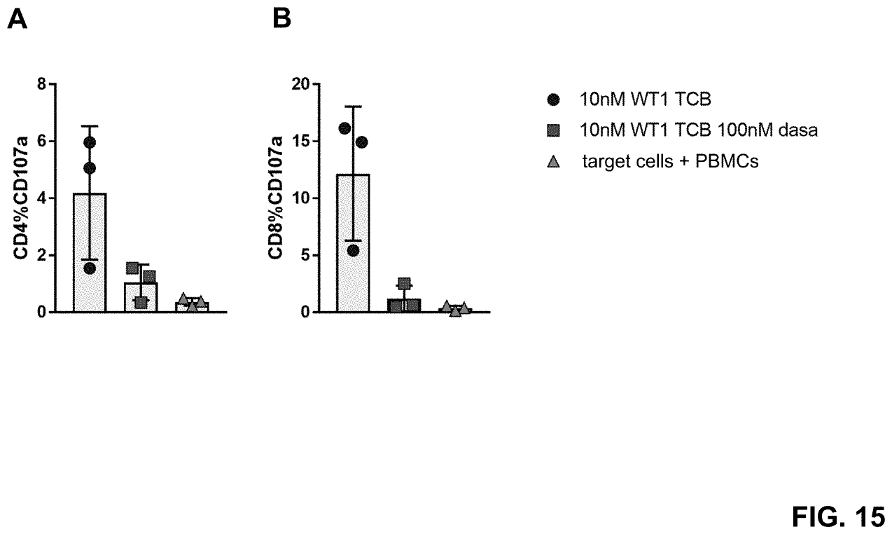

To assess whether dasatinib can efficiently prevent TCB-mediated target cell killing by activated T cells, we set up an in vitro killing assay with two stimulation steps, mimicking an ON/OFF switch. During the first stimulation, PBMCs were activated on carboxyfluorescein succinimidyl ester (CFSE)-labelled SKM-1 tumors cells with HLA-A2 WT-1-TCB in the absence of dasatinib (ON). During the second stimulation, activated PBMCs together with dead CFSE labelled SKM-1 tumors cells were washed and re-stimulated by HLA-A2 WT-1-TCB on fresh CTV labelled SKM-1 tumor cells in the presence of 100 nM dasatinib (OFF). The use of CFSE and CTV labelled SKM-1 tumors allowed to differentiate tumor cells used for first and second stimulation by flow cytometry (FIG. 8). Treatment with HLA-A2-WT-1-TCB during the first stimulation induced upregulation of early and late T cell activation markers CD69 and CD25 on CD8+ and CD4+ T cells (FIG. 9) as well as the killing of CFSE labelled SKM-1 target cells (FIG. 10 and FIG. 11). Consistently, T cells were activated and functional before the addition of dasatinib in the system. 87.6% of CTV labelled SKM-1 cells were alive upon second stimulation with 10 nM HLA-A2 WT-1 TCB in the presence of 100 nM dasatinib, but only 2.04% of CTV labelled SKM-1 tumor cells were alive upon restimulation with 10 nM HLA-A2 WT-1-TCB in the absence of dasatinib (FIG. 10). The addition of 100 nM dasatinib upon re-stimulation of activated T cells and dead CFSE labelled SKM-1 tumor cells on fresh CTV labelled SKM-1 tumor cells with HLA-A2 WT-1-TCB effectively prevented the killing of CTV labelled SKM-1 cells (ON/OFF) as opposed to the positive control (ON/ON) (FIG. 10 and FIG. 11). Additionally, T cell-derived IFN-γ and IL-2 and T cell and monocyte-derived TNF-α release were fully inhibited upon re-stimulation in the presence of 100 nM dasatinib in comparison to the positive control ON/ON (FIG. 12). This result emphasizes that dasatinib acts as a pharmacological ON/OFF switch on activated T cells, switching off T cell functionality as well as T-cell mediated target cell killing rapidly. To investigate how dasatinib could prevent T cell-cytotoxicity, we measured the expression of the degranulation marker CD107a by intracellular staining as a readout for T cell degranulation (FIG. 13) after stimulation with 10 nM HLA-A2 WT-1-TCB in the presence and absence of 100 nM dasatinib. Among CD4+ and CD8+ T cells, 16.6% and 7.53%, respectively, of the cells were positive for CD107a and only 1.22% and 2.08%, respectively, were positive for CD107a when the medium was supplemented with 100 nM dasatinib (FIG. 14). Treatment with 10 nM HLA-A2 WT-1-TCB induced CD107a expression on CD4+ and CD8+ T cells, which was prevented with the addition of 100 nM dasatinib in the assay (FIG. 15). By preventing T cell degranulation, dasatinib can restrain the release of cytotoxic granules like perforin and granzyme B responsible for the killing of tumor cells. Overall, the addition of 100 nM dasatinib upon second stimulation blocked TCB-induced T cell cytoxicity.

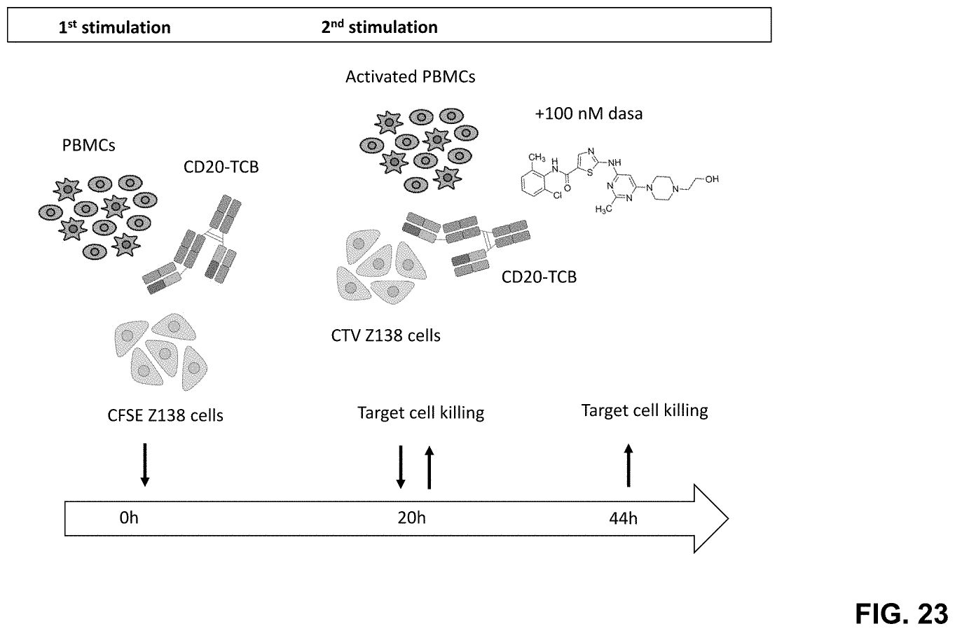

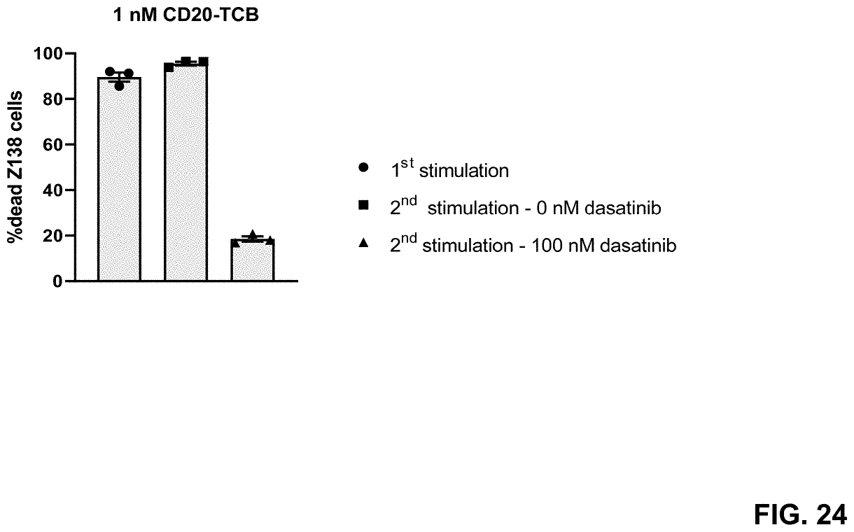

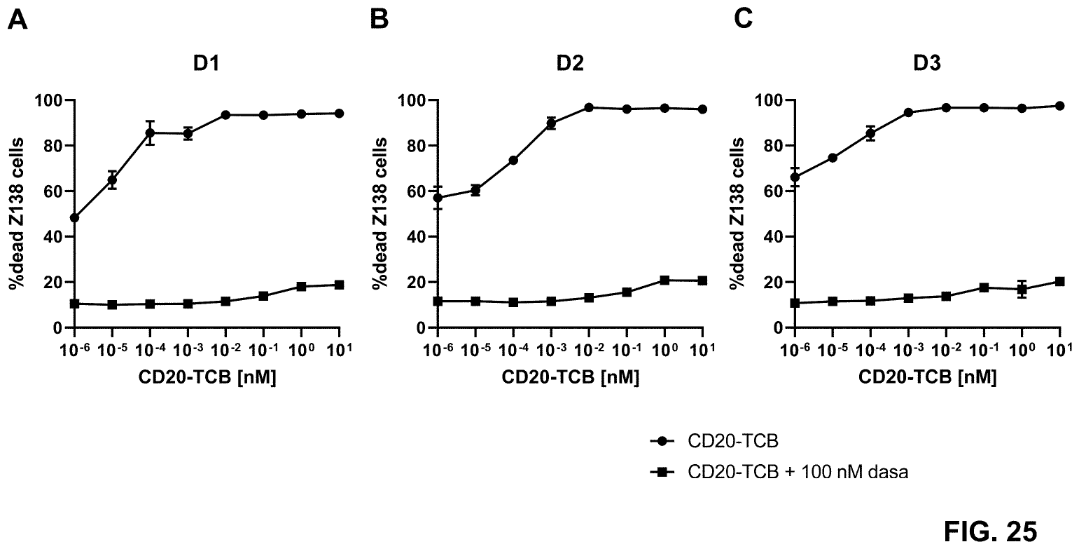

A similar re-stimulation experiment was performed with a different TCB, targeting CD20 (CD20-TCB (SEQ ID NOs 28-35, 48-59), 1 nM), and the killing of Z138 target cells measured after the first and second stimulation (FIG. 23). As seen with HLA-A2 WT-1 TCB, in this experiment the addition of 100 nM dasatinib upon re-stimulation of activated T cells and dead CFSE labelled target cells on fresh CTV labelled target cells with CD20-TCB effectively prevented the killing of CTV labelled target cells (ON/OFF) as opposed to the positive control (ON/ON) (FIG. 24 and FIG. 25).

### Example 4. Dasatinib Reversibly Stops TCB-Induced T Cell Activation

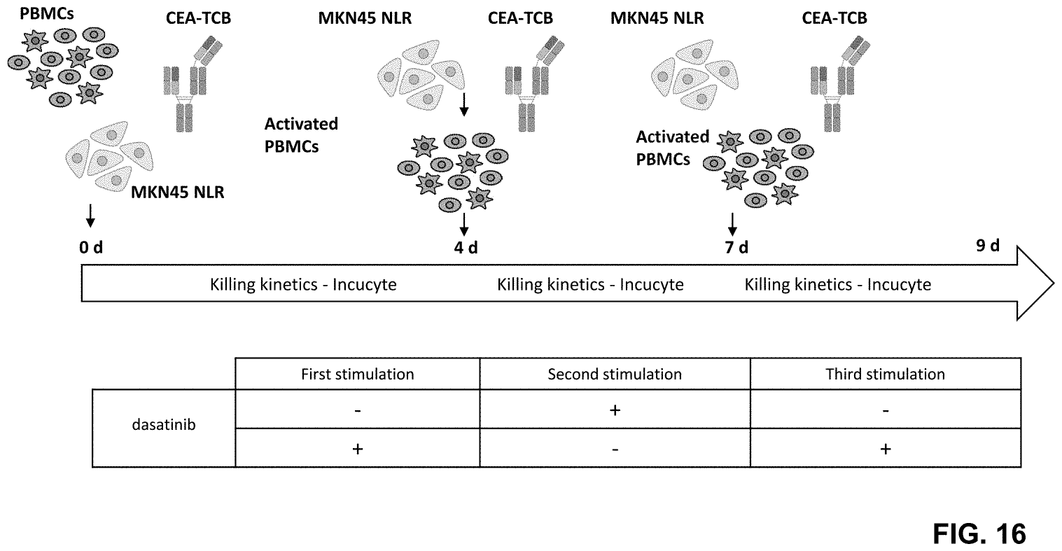

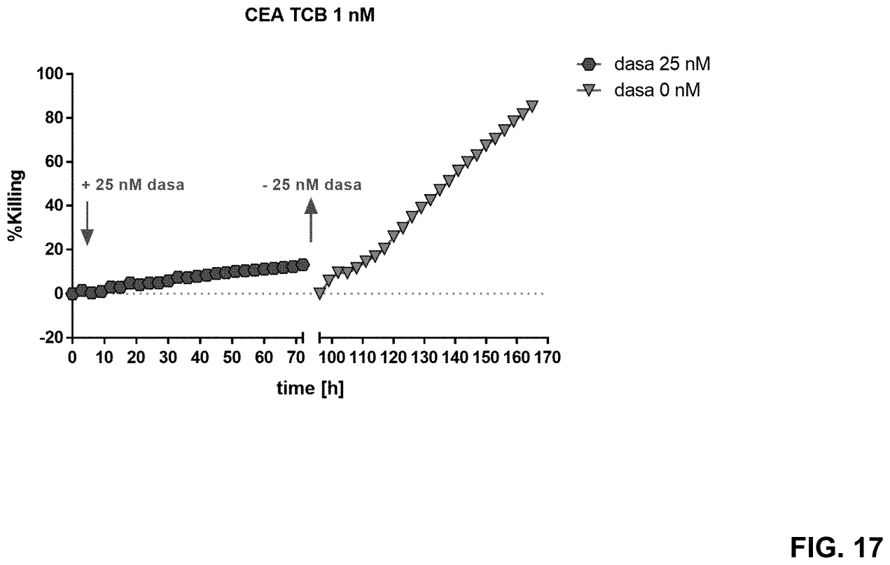

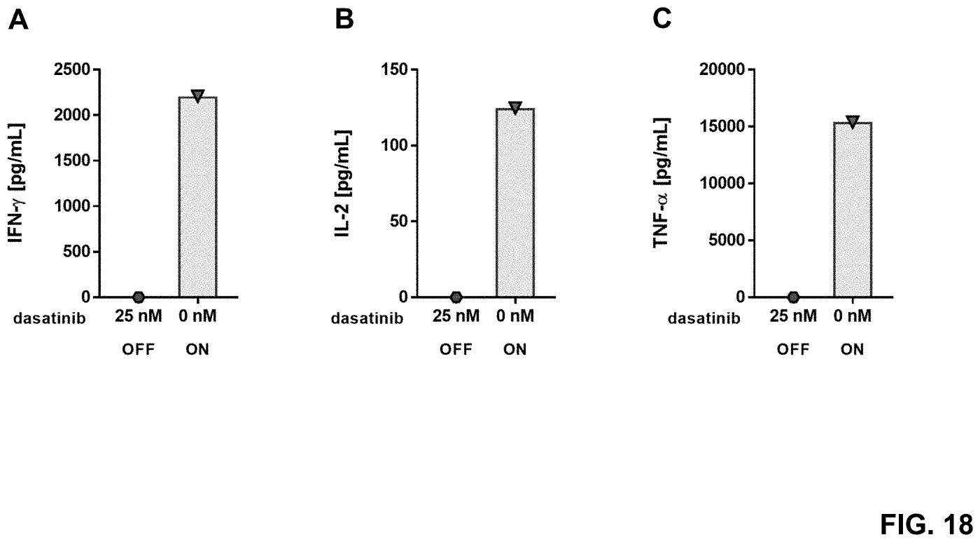

We then verified if dasatinib's effect is reversible upon its removal. Therefore we set-up a killing assay with two and three stimulations in the presence and absence of dasatinib and followed the killing kinetics using an Incucyte® system (FIG. 16). After each stimulation, effector cells were washed and re-stimulated on fresh MKN45 NLR target cells with 1 nM CEA TCB in the presence and absence of dasatinib allowing to mimic OFF/ON switch and ON/OFF/ON switch. When 25 nM dasatinib was added during the first stimulation, it resulted in killing inhibition which was then reversed upon dasatinib removal for the second stimulation (OFF/ON) (FIG. 17). IFN-γ, IL-2 and TNF-α were not found in the supernatant after the first stimulation with 1 nM CEA-TCB in the presence of 25 nM dasatinib indicating full inhibition of T-cell derived cytokine release in the presence of dasatinib. However, removal of 25 nM dasatinib and restimulation with 1 nM CEA-TCB resulted in the release of IFN-γ, IL-2 and TNF-α, indicating that T cell functionality was restored upon dasatinib removal (FIG. 18).

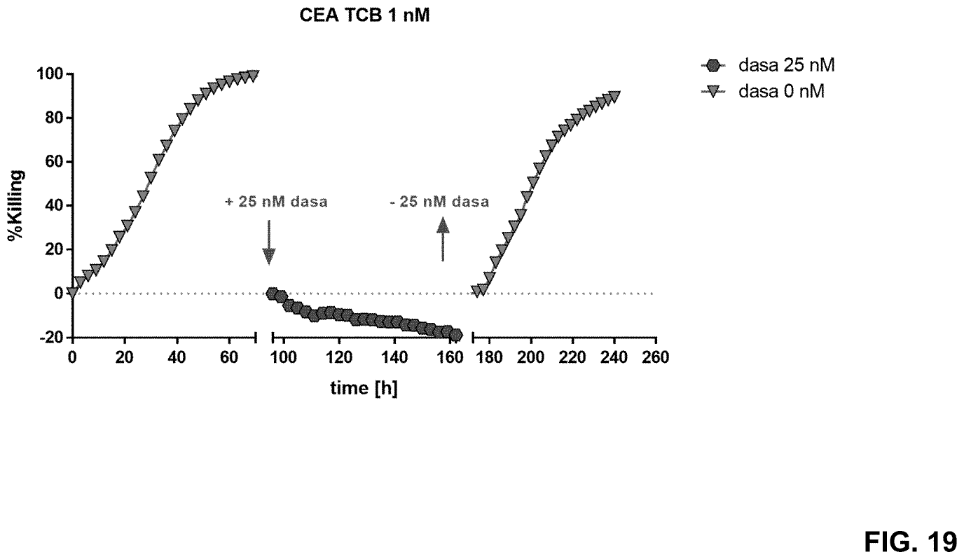

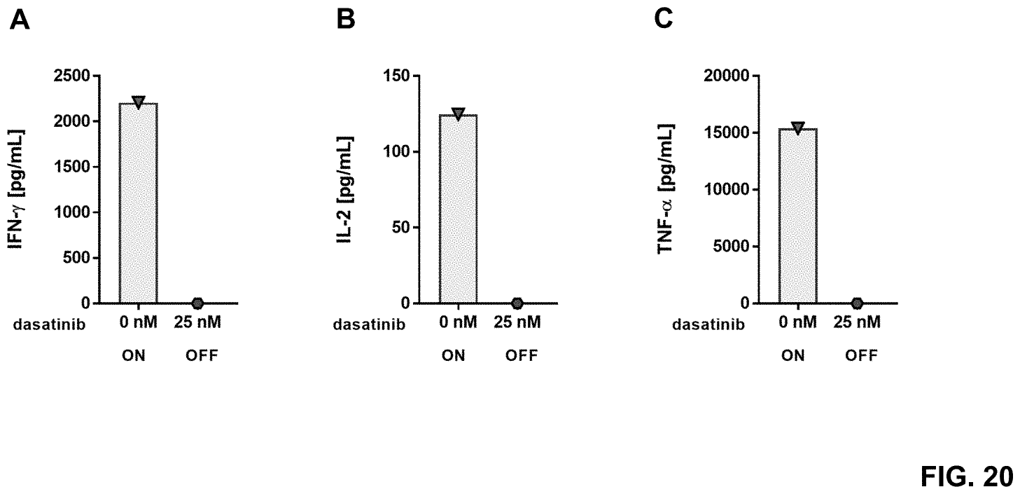

Lastly, we evaluated if the effect of dasatinib was reversible on activated T cells. Consequently, we supplemented the media with 25 nM dasatinib upon the second stimulation with 1 nM CEA-TCB to prevent killing from activated T cells and then removed the dasatinib upon third stimulation with 1 nM CEA-TCB to verify if killing would be restored. After the first stimulation, 1 nM CEA-TCB triggered the killing of MKN45 cells, which was then inhibited with the addition of 25 nM upon second stimulation and restored upon third stimulation with the removal of dasatinib (FIG. 19). Addition of 25 nM dasatinib in the killing assay also prevented the release of IFN-γ, IL-2 and TNF-α (FIG. 20). We concluded that the effect of dasatinib in the prevention of T cell activation and cytotoxicity is reversible.

### Example 5. Low Doses of Dasatinib Equilibrate Cytokine Release Upon First and Second Stimulation with TCBs

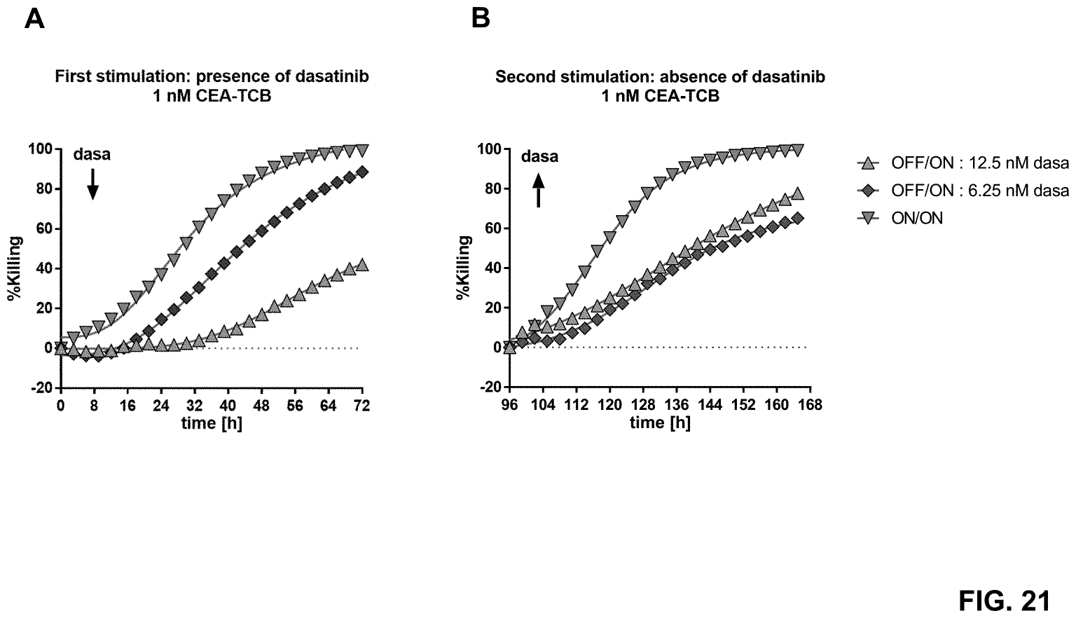

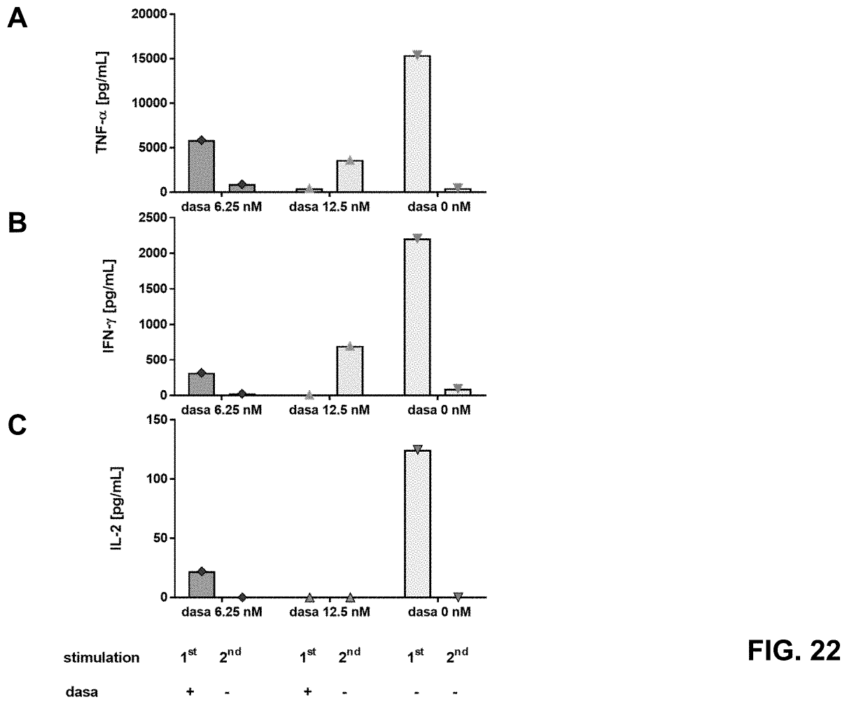

As shown in FIG. 21, a dasatinib concentration of 12.5 nM and 6.25 nM did not result in full killing inhibition but decreased TCB-induced cytokine release. For T-cell engaging antibodies, cytokine release peaks are higher upon the first treatment than after subsequent treatments. We were curious to see if low doses of dasatinib could prevent cytokine release while minimally affecting TCB-efficacy upon first stimulation. In killing assays as described in FIG. 16, the cytokine levels upon first treatment with 1 nM CEA-TCB in the presence of 6.25 nM and 12.5 nM dasatinib and upon second treatment with 1 nM CEA-TCB in the absence of dasatinib were measured in the supernatants. The presence of 6.25 nM and 12.5 nM dasatinib during the first stimulation lowered the release of IFN-γ, IL-2 and TNF-α (FIG. 22) while it only partially inhibited the killing induced by 1 nM CEA-TCB (FIG. 21A). In agreement with the reversibility properties of dasatinib, killing was restored upon the removal of dasatinib during second stimulation (FIG. 21B), while levels of TNF-α, IL-2 and IFN-γ remained low (FIG. 22). This data suggests that low dose of dasatinib may prevent TCB-induced cytokine release triggered upon first treatment with TCB. Its removal upon second stimulation may also balance the cytokine release and restore TCB-induced cytotoxicity due to the reversibility properties of dasatinib.

### Example 6. Dasatinib Prevents CD19-TCB-Induced T Cell Cytotoxicity, T Cell Activation and Cytokine Release In Vitro

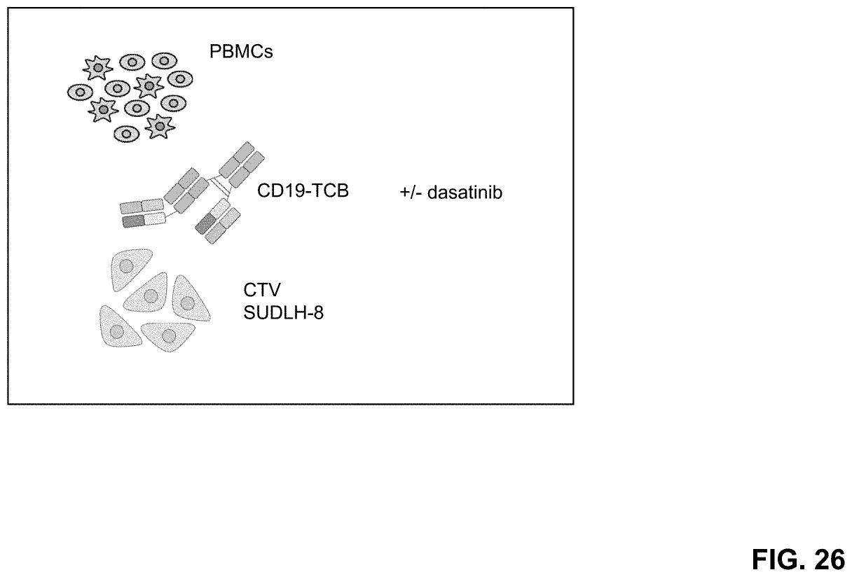

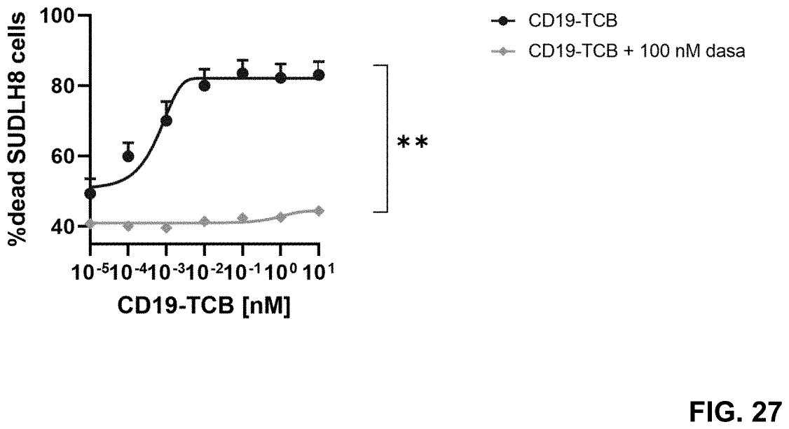

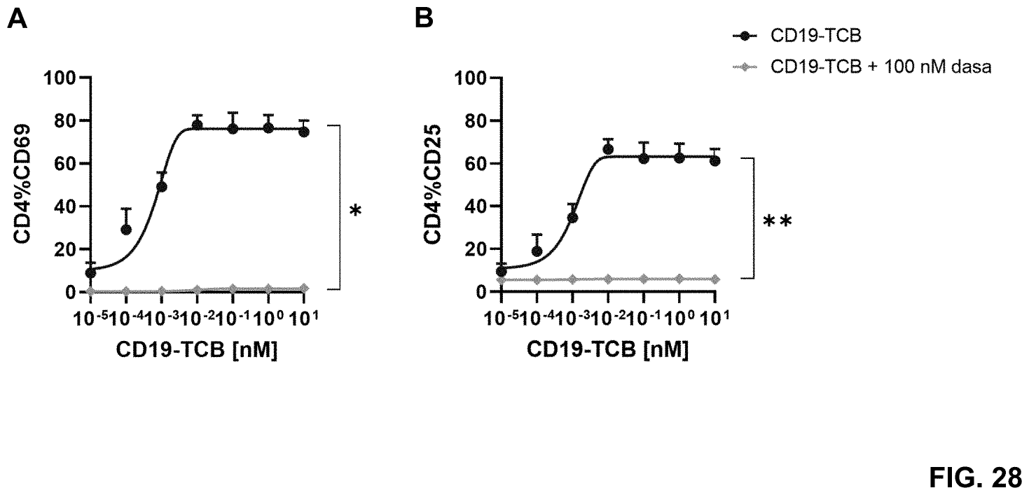

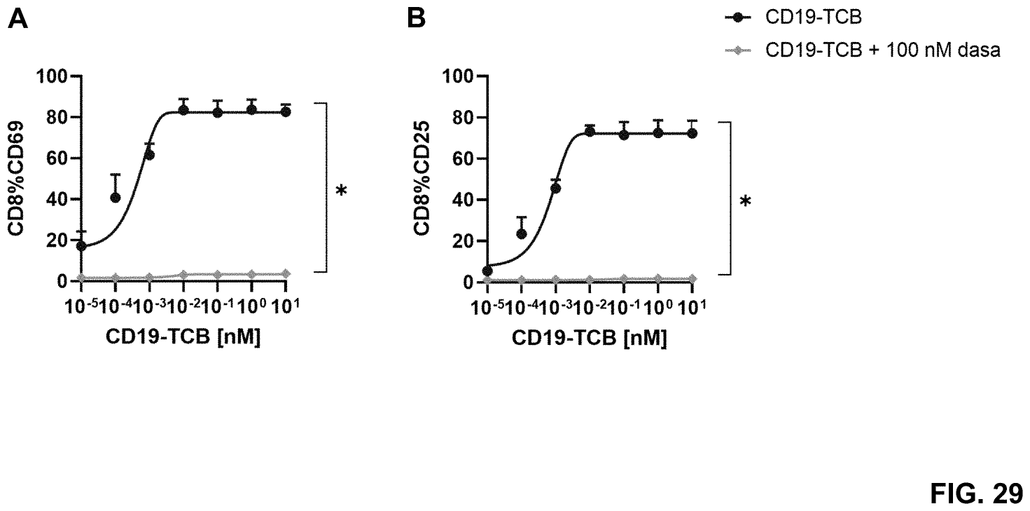

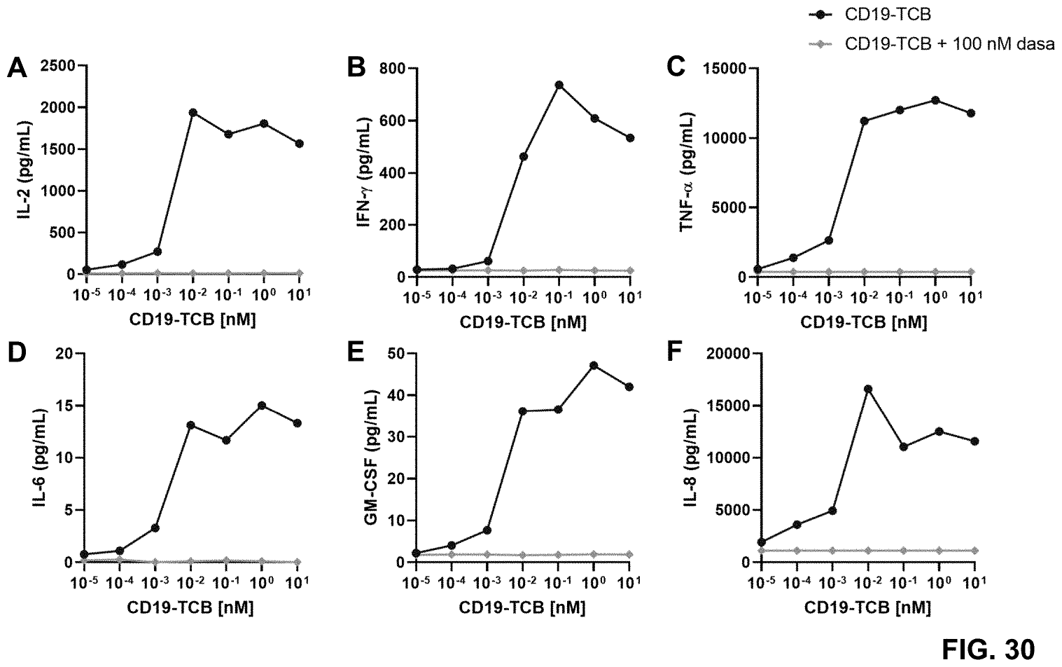

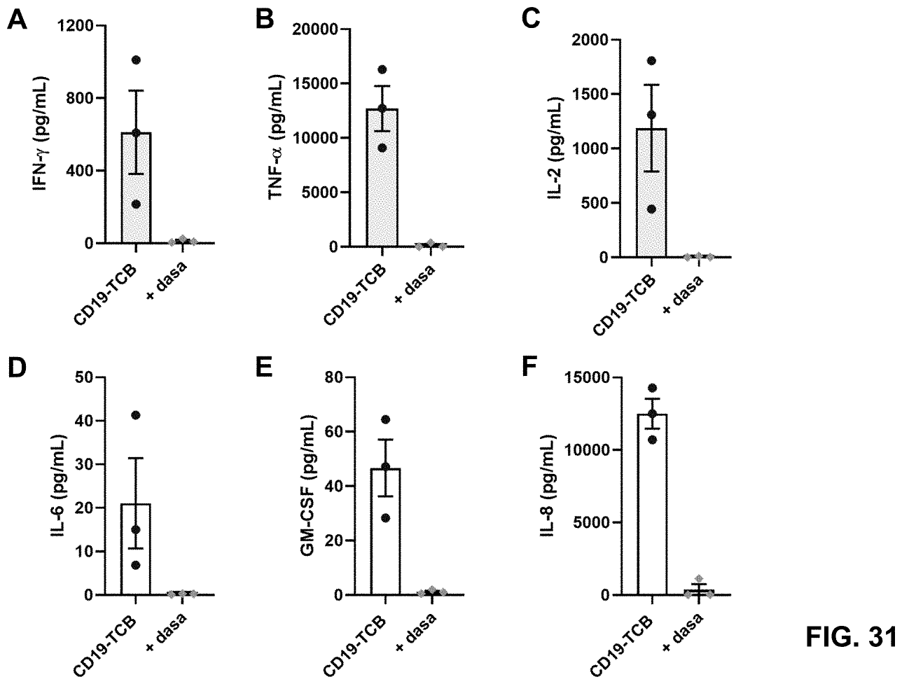

To assess whether dasatinib can prevent T cell cytotoxicity, T cell activation and cytokine release induced by another TCB, CD19-TCB (SEQ ID NOs 29, 31-33, 35, 64-74, 76-78, 80), PBMCs were co-cultured together with CellTraceViolet® (CTV) labelled SUDLH-8 cells and escalating doses of CD19-TCB in the absence and presence of 100 nM dasatinib (FIG. 26). The killing of CTV labelled SUDLH-8 cells was measured by flow cytometry using a Live/Dead Near Infra Red (NIR) dye. As a result, the addition of 100 nM dasatinib prevented killing of SUDLH-8 tumor cells by CD19-TCB (FIG. 27). The expression of CD69 and CD25 were measured on CD4+ (FIG. 28) and CD8+(FIG. 29) T cells by flow cytometry as a readout for T cell activation. The addition of 100 nM dasatinib prevented CD4+ and CD8+ T cell activation. Lastly, the cytokine levels were analyzed in the supernatants of the assay by Luminex to evaluate the effect of dasatinib on CD19-TCB-induced cytokine release (FIGS. 30 and 31). In line with killing and T cell activation data, dasatinib prevented the release of IL-2 (FIG. 30A, 31C), IFN-γ (FIG. 30B, 31A), TNF-α (FIG. 30C, 31B), IL-6 (FIG. 30D, 31D), GM-CSF (FIG. 30E, 31E) and IL-8 (FIG. 30F, 31F).

Altogether, these in vitro data suggest that dasatinib efficiently prevents CD19-TCB-induced T cell cytotoxicity, T cell activation and cytokine release.

### Example 7. Dasatinib Prevents CD19-TCB-Induced T Cell Cytotoxicity, T Cell Activation and Cytokine Release In Vivo

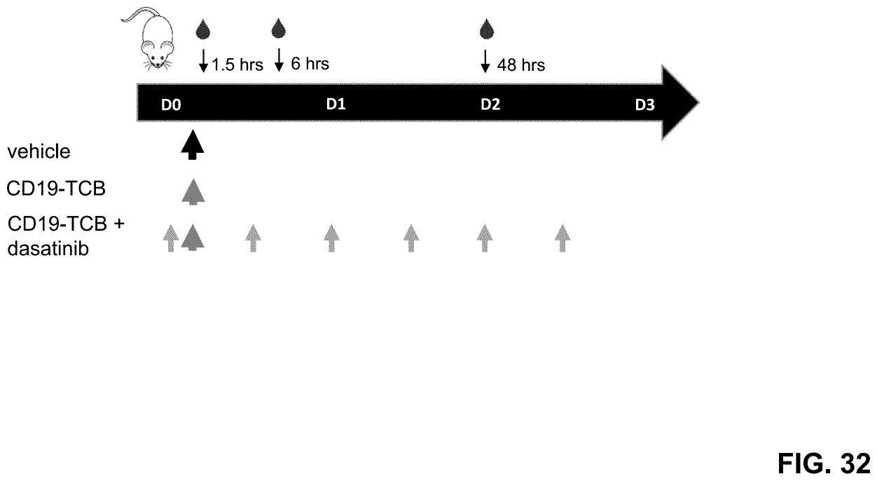

To verify whether dasatinib can prevent CD19-TCB-induced B cell depletion and cytokine release in vivo, humanized NSG mice were either treated with 0.5 mg/kg CD19-TCB or co-treated with 0.5 mg/kg CD19-TCB and 50 mg/kg dasatinib as illustrated in FIG. 32. To best mimick the pharmacodynamic profile of dasatinib in the clinic and to verify whether the resulting exposure would be sufficient to prevent CD19-TCB-induced T cell cytotoxicity and cytokine release, dasatinib was given per os twice per day.

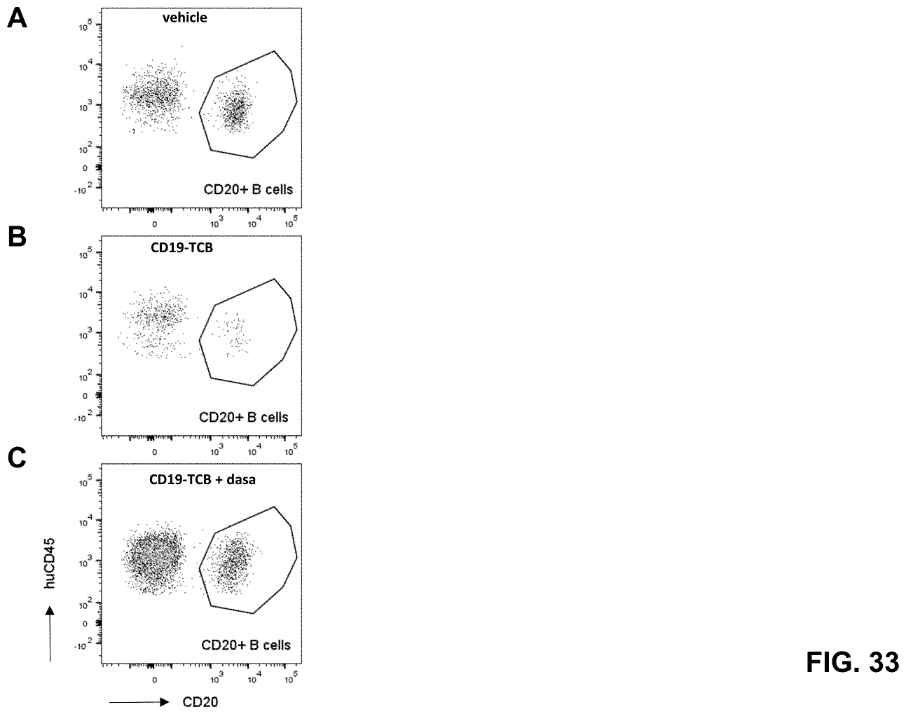

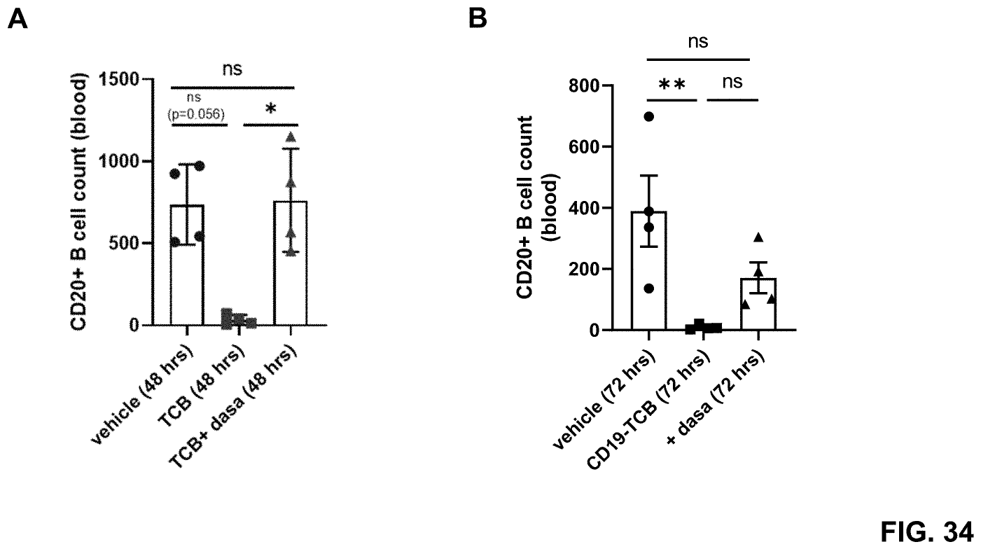

At 48 hrs and 72 hrs, mice were bled and the CD20+ B cell count was measured by flow cytometry (FIG. 33). As a result, dasatinib prevented killing of CD20+ B cells by CD19-TCB at 48 hrs (FIG. 34), however the killing was partially restored at 72 hrs (FIG. 34). The half-life of dasatinib in blood of mice being around 6 hrs, the exposure of dasatinib was probably not sufficient to durably prevent CD19-TCB-induced T cell cytotoxicity, leading to partial activity of CD19-TCB. In line with the in vitro observations, these data suggest that the inhibitory effect of dasatinib in vivo is reversible.

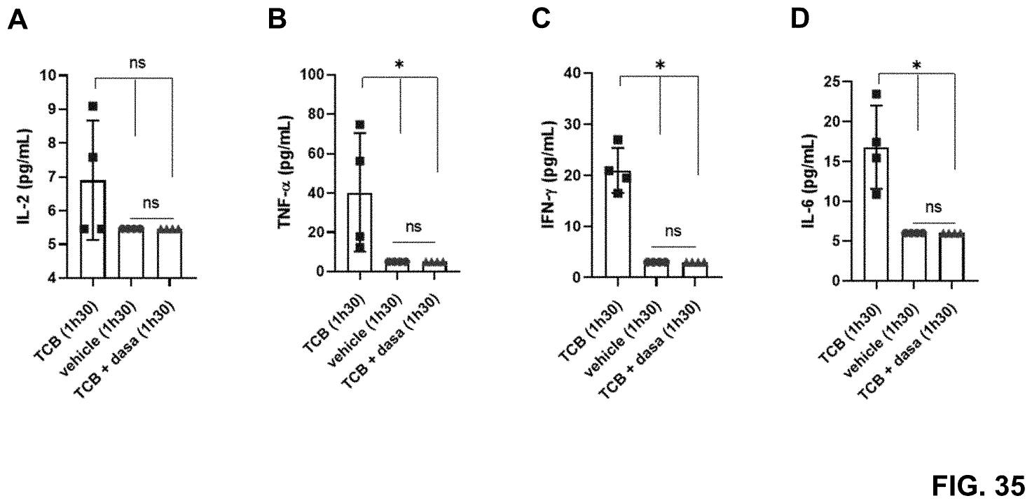

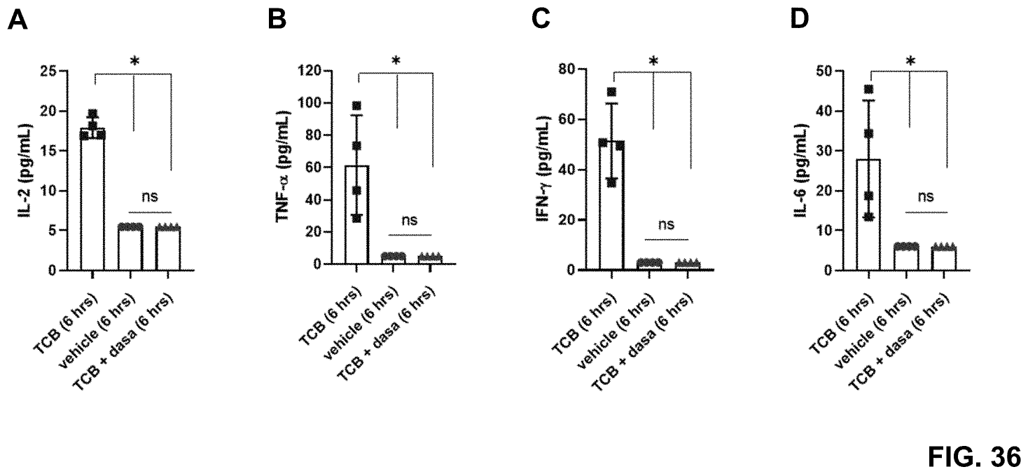

Lastly, mice were bled 1.5 and 6 hrs post treatment with CD19-TCB and dasatinib to collect serum for cytokine measurements by Luminex (FIGS. 35 and 36). At both timepoints, dasatinib inhibited CD19-TCB-induced IL-2 (FIG. 35A, 36A), TNF-α (FIG. 35B, 36B), IFN-γ (FIG. 35C, 36C) and IL-6 (FIG. 35D, 36D), indicating that dasatinib rapidly switched off T cell-derived cytokine release by CD19-TCB.

In line with the in vitro findings, the rapid onset of the activity of dasatinib allows to prevent B cell depletion and cytokine release induced by the first infusion of CD19-TCB in humanized NSG mice. Collectively, these data demonstrate the favorable pharmacodynamic profile of dasatinib to prevent CD19-TCB induced T cell cytotoxicity and cytokine release for up to 48 hrs when given twice per day, as well as the reversibility of the inhibitory effect of dasatinib.

### Example 8. Prophylactic Use of Dasatinib Strongly Prevents TCB-Mediated Cytokine Release while Retaining Anti-Tumor Efficacy

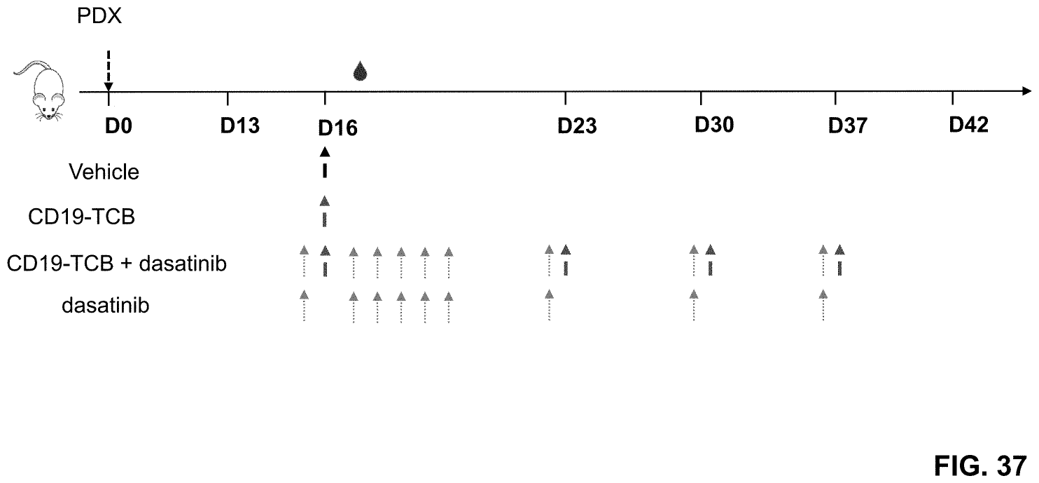

We evaluated the impact of transient interventions with dasatinib on CD19-TCB anti-tumor activity in humanized NSG mice engrafted with a lymphoma patient derived xenograft (PDX). Therefore, mice were treated with either vehicle, 0.5 mg/kg CD19-TCB as a monotherapy, or 20 mg/kg dasatinib, alone or in combination with 0.5 mg/kg CD19-TCB (FIG. 37). Dasatinib was given one hour prior and 6 hrs after the first treatment with CD19-TCB and then twice per day for the next 2 days to prevent cytokine release, predominantly occurring upon the first infusion. Moreover, dasatinib was also administered one hour before each subsequent treatment to prevent eventual residual cytokine secretion (FIG. 37).

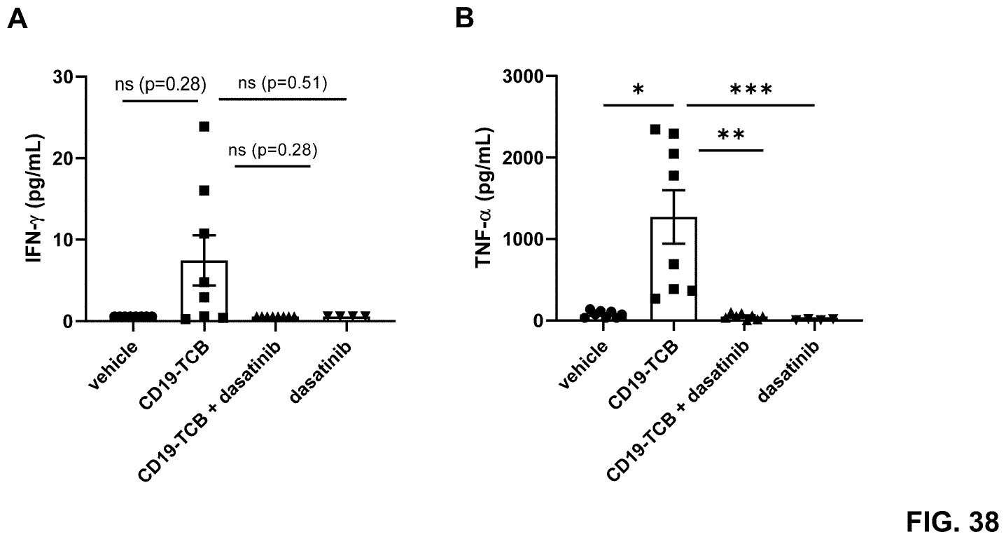

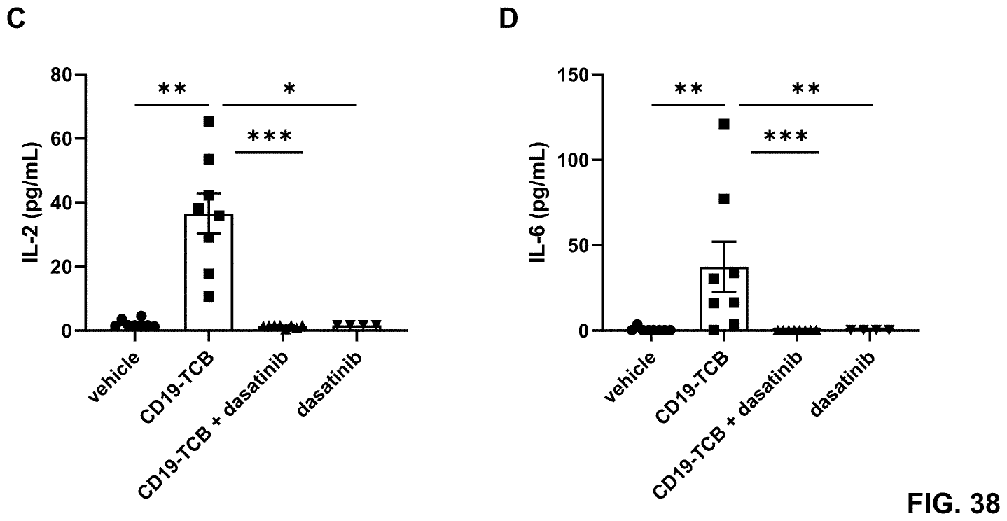

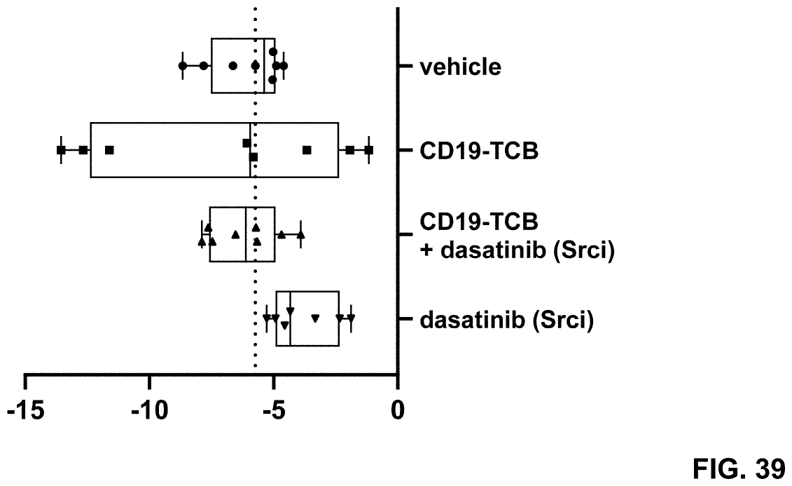

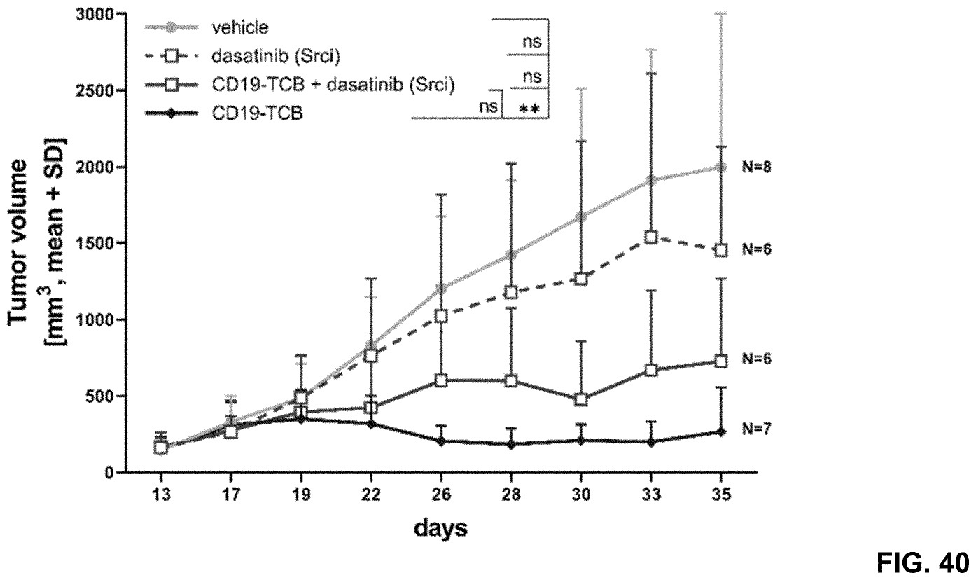

As indicated by the levels of IFN-γ, TNF-α, IL-2 and IL-6 in FIG. 38, dasatinib strongly reduced CD19-TCB-mediated cytokine release upon the first infusion. The reduction of cytokine levels was associated with a milder body weight change 24 hrs after the first CD19-TCB treatment (FIG. 39), suggesting that dasatinib may efficiently prevent CRS symptoms. Besides, the transient use of dasatinib minimally, yet not significantly, interfered with anti-tumor efficacy as shown by the tumor growth curves in FIG. 40.

Due to the combination of the short PK/PD properties of dasatinib with longer PK/PD properties of CD19-TCB, and in agreement with its reversible inhibitory properties, dasatinib strongly reduced cytokine release after the first infusion while retaining CD19-TCB anti-tumor efficacy. As a result, CD19-TCB was better tolerated and remained efficacious, suggesting that transient prophylactic use of dasatinib in the clinic may prevent incidence of CRS upon the first infusion with TCB.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1.-2. (canceled)

3. A method for treatment of a disease in an individual, wherein said method comprises
(a) the administration of a T cell bispecific antibody to the individual, and
(b) the administration of a tyrosine kinase inhibitor (TKI) to the individual for the prevention or mitigation of an adverse effect related to the administration of the T cell bispecific antibody.

4.-5. (canceled)

6. A method for preventing or mitigating an adverse effect related to the administration of a T cell bispecific antibody to an individual, comprising the administration of a tyrosine kinase inhibitor (TKI) to the individual.

7.-60. (canceled)

61. The method of claim 6, wherein the TKI is a Lck or Src kinase inhibitor.

62. The method of claim 61, wherein the TKI is dasatinib.

63. The method of claim 6, wherein administration of the TKI inhibits at least one activity induced by the T cell bispecific antibody.

64. The method of claim 63, wherein the TKI inhibits at least one activity induced by the T cell bispecific antibody selected from:
(i) T cell activation,
(ii) T cell proliferation,
(iii) T cell cytotoxicity,
(iv) T cell receptor signaling, and
(v) T cell cytokine secretion.

65. The method of claim 64, wherein the TKI inhibits T cell cytokine secretion induced by the T cell bispecific antibody, and wherein said cytokine is at least one of IL-2, TNF-α, IFN-γ, IL-6, or IL-1β.

66. The method of claim 64, wherein the T cells are CD8+ T cells or CD4+ cells.

67. The method of claim 63, wherein the inhibition by the TKI is reversible.

68. The method of claim 6, wherein administration of the TKI reduces a serum level of one or more cytokines in the individual.

69. The method of claim 68, wherein the one or more cytokine having a reduced serum level is at least one of IL-2, TNF-α, IFN-γ, IL-6, or IL-1β.

70. The method of claim 6, wherein the adverse effect is at least one of (i) cytokine release syndrome (CRS), (ii) fever, hypotension or hypoxia, or (iii) an elevated serum level of one or more cytokines.

71. The method of claim 70, wherein the one or more cytokine having an elevated serum level is at least one of IL-2, TNF-α, IFN-γ, IL-6, or IL-1β.

72. The method of claim 6, wherein administration of the TKI is upon manifestation of the adverse effect in the individual.

73. The method of claim 6, wherein administration of the TKI is
(i)
(a) before,
(b) concurrent to, or
(c) after
the administration of the T cell bispecific antibody;
(ii) intermittently or continuously, or
(iii) orally.

74. The method of claim 6, wherein administration of the TKI is at a dose sufficient to reduce the serum level of one of more cytokines in the individual.

75. The method of claim 74, wherein administration of the TKI is at a dose insufficient to inhibit T cell activation or T cell cytotoxicity.

76. The method of claim 6, wherein administration of the TKI is at an effective dose.

77. The method of claim 76, wherein the effective dose of the TKI is about 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg.

78. The method of claim 77, wherein the effective dose of the TKI is about 10 mg to about 100 mg.

79. The method of claim 6, wherein the TKI is administered until the adverse effect is prevented or mitigated.

80. The method of claim 79, wherein the TKI is administered prior to, concurrent with, or subsequent to a first administration of the T cell bispecific antibody.

81. The method of claim 6, wherein the T cell bispecific antibody is administered
(i) at an effective dose,
(ii) parenterally, or
(iii) as the first administration of the T cell bispecific antibody to the individual.

82. The method of claim 6, wherein administering the T cell bispecific antibody induces
(i) T cell activation,
(ii) T cell proliferation,
(iii) T cell cytotoxicity,
(iv) T cell receptor signaling, or
(v) T cell cytokine secretion.

83. The method of claim 82 wherein administering the T cell bispecific antibody induces T cell cytokine secretion, and wherein said cytokine is at least one of IL-2, TNF-α, IFN-γ, IL-6, or IL-1β.

84. The method of claim 82, wherein the T cells are CD8+ T cells or CD4+ cells.

85. The method of claim 6, wherein the T cell bispecific antibody comprises an antigen binding moiety that binds to CD3 and an antigen binding moiety that binds to a target cell antigen.

86. The method of claim 85, wherein the target cell antigen is carcinoembryonic antigen (CEA).

87. The method of claim 86, wherein the T cell bispecific antibody comprises
(i) a first antigen binding moiety that binds to CD3 comprising
(a) a heavy chain variable region comprising a heavy chain CDR (HCDR) 1 of SEQ ID NO: 28, a HCDR2 of SEQ ID NO: 29, and a HCDR3 of SEQ ID NO: 30, and
(b) light chain variable region comprising a light chain CDR (LCDR) 1 of SEQ ID NO: 31, a LCDR2 of SEQ ID NO: 32 and a LCDR3 of SEQ ID NO: 33, and
(ii) a second antigen binding moiety that binds to CEA comprising
(a) a heavy chain variable region comprising a heavy chain CDR (HCDR) 1 of SEQ ID NO: 36, a HCDR2 of SEQ ID NO: 37, and a HCDR3 of SEQ ID NO: 38, and
(b) a light chain variable region comprising a light chain CDR (LCDR) 1 of SEQ ID NO: 39, a LCDR2 of SEQ ID NO: 40 and a LCDR3 of SEQ ID NO: 41.

88. The method of claim 87, wherein the T cell bispecific antibody further comprises (i) a third antigen binding moiety that binds to CEA, (ii) an Fc domain composed of a first and a second subunit, or (iii) both (i) and (ii).

89. The method of claim 88, wherein the T cell bispecific antibody comprises
(i) a first antigen binding moiety that binds to CD3, comprising a heavy chain variable region comprising a heavy chain CDR (HCDR) 1 of SEQ ID NO: 28, a HCDR2 of SEQ ID NO: 29, and a HCDR3 of SEQ ID NO: 30; and a light chain variable region comprising a light chain CDR (LCDR) 1 of SEQ ID NO: 31, a LCDR2 of SEQ ID NO: 32 and a LCDR3 of SEQ ID NO: 33, wherein the first antigen binding moiety is a crossover Fab molecule wherein either the variable or the constant regions of the Fab light chain and the Fab heavy chain are exchanged;
(ii) a second and a third antigen binding moiety that bind to CEA, comprising a heavy chain variable region comprising a heavy chain CDR (HCDR) 1 of SEQ ID NO: 36, a HCDR2 of SEQ ID NO: 37, and a HCDR3 of SEQ ID NO: 38; and a light chain variable region comprising a light chain CDR (LCDR) 1 of SEQ ID NO: 39, a LCDR2 of SEQ ID NO: 40 and a LCDR3 of SEQ ID NO: 41; wherein the second and third antigen binding moiety are each a Fab molecule; and
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

90. The method of claim 89, wherein (a) the first antigen binding moiety of the T cell bispecific antibody comprises a heavy chain variable region sequence that is at least about 95%, 96%, 97%, 98%, 99% or 100% identical to the amino acid sequence of SEQ ID NO: 34 and a light chain variable region sequence that is at least about 95%, 96%, 97%, 98%, 99% or 100% identical to the amino acid sequence of SEQ ID NO: 35, or the (b) second or (c) third antigen binding moieties of the T cell bispecific antibody comprise a heavy chain variable region sequence that is at least about 95%, 96%, 97%, 98%, 99% or 100% identical to the amino acid sequence of SEQ ID NO: 42 and a light chain variable region sequence that is at least about 95%, 96%, 97%, 98%, 99% or 100% identical to the amino acid sequence of SEQ ID NO: 43, or any combination of (a), (b), and (c).

91. The method of claim 89, wherein the Fc domain of the T cell bispecific antibody comprises (i) a modification promoting the association of the first and the second subunit of the Fc domain, (ii) one or more amino acid substitution that reduces binding to an Fc receptor and/or effector function, or (iii) both (i) and (ii).

92. The method of claim 6, wherein the T cell bispecific antibody is cibisatamab.

93. The method of claim 85, wherein the target cell antigen is HLA-2/WT1.

94. The method of claim 93, wherein the T cell bispecific antibody comprises
(i) a first antigen binding moiety that binds to CD3 comprising
(a) a heavy chain variable region comprising a heavy chain CDR (HCDR) 1 of SEQ ID NO: 1, a HCDR2 of SEQ ID NO: 2, and a HCDR3 of SEQ ID NO: 3, and
(b) a light chain variable region comprising a light chain CDR (LCDR) 1 of SEQ ID NO: 4, a LCDR2 of SEQ ID NO: 5 and a LCDR3 of SEQ ID NO: 6, and
(ii) a second antigen binding moiety that binds to HLA-A2/WT1 comprising
(a) a heavy chain variable region comprising a heavy chain CDR (HCDR) 1 of SEQ ID NO: 9, a HCDR2 of SEQ ID NO: 10, and a HCDR3 of SEQ ID NO: 11, and
(b) a light chain variable region comprising a light chain CDR (LCDR) 1 of SEQ ID NO: 12, a LCDR2 of SEQ ID NO: 13 and a LCDR3 of SEQ ID NO: 14.

95. The method of claim 94, wherein the T cell bispecific antibody comprises (i) a third antigen binding moiety that binds to HLA-A2/WT1, (ii) an Fc domain composed of a first and a second subunit, or (iii) both (i) and (ii).

96. The method of claim 95, wherein the T cell bispecific antibody comprises
(i) a first antigen binding moiety that binds to CD3, comprising a heavy chain variable region comprising a heavy chain CDR (HCDR) 1 of SEQ ID NO: 1, a HCDR2 of SEQ ID NO: 2, and a HCDR3 of SEQ ID NO: 3; and a light chain variable region comprising a light chain CDR (LCDR) 1 of SEQ ID NO: 4, a LCDR2 of SEQ ID NO: 5 and a LCDR3 of SEQ ID NO: 6, wherein the first antigen binding moiety is a crossover Fab molecule wherein either the variable or the constant regions of the Fab light chain and the Fab heavy chain are exchanged;
(ii) a second and a third antigen binding moiety that bind to HLA-A2/WT1, comprising a heavy chain variable region comprising a heavy chain CDR (HCDR) 1 of SEQ ID NO: 9, a HCDR2 of SEQ ID NO: 10, and a HCDR3 of SEQ ID NO: 11; and a light chain variable region comprising a light chain CDR (LCDR) 1 of SEQ ID NO: 12, a LCDR2 of SEQ ID NO: 13 and a LCDR3 of SEQ ID NO: 14, wherein the second and third antigen binding moiety are each a Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

97. The method of claim 96, wherein the (a) first antigen binding moiety of the T cell bispecific antibody comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8, or the (b) second or (c) third antigen binding moieties of the T cell bispecific antibody comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16, or any combination of (a), (b), and (c).

98. The method of claim 96, wherein the first antigen binding moiety of the T cell bispecific antibody is a crossover Fab molecule wherein the variable regions of the Fab light chain and the Fab heavy chain are exchanged, and wherein the second and third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

99. The method of claim 96, wherein the Fc domain of the T cell bispecific antibody comprises (i) a modification promoting the association of the first and the second subunit of the Fc domain, (ii) one or more amino acid substitution that reduces binding to an Fc receptor or effector function, or (iii) both (i) and (ii).

100. The method of claim 85, wherein the target cell antigen is CD20.

101. The method of claim 100, wherein the T cell bispecific antibody comprises
(i) a first antigen binding moiety that binds to CD3 and comprises
(a) a heavy chain variable region comprising a heavy chain CDR (HCDR) 1 of SEQ ID NO: 28, a HCDR2 of SEQ ID NO: 29, and a HCDR3 of SEQ ID NO: 30, and
(b) a light chain variable region comprising a light chain CDR (LCDR) 1 of SEQ ID NO: 31, a LCDR2 of SEQ ID NO: 32 and a LCDR3 of SEQ ID NO: 33, and
(ii) a second antigen binding moiety that binds to CD20 and comprises
(a) a heavy chain variable region comprising a heavy chain CDR (HCDR) 1 of SEQ ID NO: 48, a HCDR2 of SEQ ID NO: 49, and a HCDR3 of SEQ ID NO: 50, and
(b) a light chain variable region comprising a light chain CDR (LCDR) 1 of SEQ ID NO: 51, a LCDR2 of SEQ ID NO: 52 and a LCDR3 of SEQ ID NO: 53.

102. The method of claim 101, wherein the T cell bispecific antibody further comprises (i) a third antigen binding moiety that binds to CD20, (ii) an Fc domain composed of a first and a second subunit, or (iii) both (i) and (ii).

103. The method of claim 102, wherein the T cell bispecific antibody comprises
(i) a first antigen binding moiety that binds to CD3, comprising
(a) a heavy chain variable region comprising a heavy chain CDR (HCDR) 1 of SEQ ID NO: 28, a HCDR2 of SEQ ID NO: 29, and a HCDR3 of SEQ ID NO: 30;
(b) a light chain variable region comprising a light chain CDR (LCDR) 1 of SEQ ID NO: 31, a LCDR2 of SEQ ID NO: 32 and a LCDR3 of SEQ ID NO: 33,
wherein the first antigen binding moiety is a crossover Fab molecule wherein either the variable or the constant regions of the Fab light chain and the Fab heavy chain are exchanged;
(ii) a second and a third antigen binding moiety that bind to CD20, comprising
(a) a heavy chain variable region comprising a heavy chain CDR (HCDR) 1 of SEQ ID NO: 48, a HCDR2 of SEQ ID NO: 49, and a HCDR3 of SEQ ID NO: 50; and
(b) a light chain variable region comprising a light chain CDR (LCDR) 1 of SEQ ID NO: 51, a LCDR2 of SEQ ID NO: 52 and a LCDR3 of SEQ ID NO: 53,
wherein the second and third antigen binding moiety are each a Fab molecule; and
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

104. The method of claim 103, wherein the (a) first antigen binding moiety of the T cell bispecific antibody comprises a heavy chain variable region sequence that is at least about 95%, 96%, 97%, 98%, 99% or 100% identical to the amino acid sequence of SEQ ID NO: 34 and a light chain variable region sequence that is at least about 95%, 96%, 97%, 98%, 99% or 100% identical to the amino acid sequence of SEQ ID NO: 35, or the (b) second or (c) third antigen binding moieties of the T cell bispecific antibody comprise a heavy chain variable region sequence that is at least about 95%, 96%, 97%, 98%, 99% or 100% identical to the amino acid sequence of SEQ ID NO: 54 and a light chain variable region sequence that is at least about 95%, 96%, 97%, 98%, 99% or 100% identical to the amino acid sequence of SEQ ID NO: 55, or any combination of (a), (b), and (c).

105. The method of claim 103, wherein the first antigen binding moiety of the T cell bispecific antibody is a crossover Fab molecule wherein the variable regions of the Fab light chain and the Fab heavy chain are exchanged, and wherein the second and third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

106. The method of claim 103, wherein the Fc domain of the T cell bispecific antibody comprises (i) a modification promoting the association of the first and the second subunit of the Fc domain, (ii) one or more amino acid substitution that reduces binding to an Fc receptor or effector function, or (iii) both (i) and (ii).

107. The method of claim 85, wherein the target cell antigen is CD19.

108. The method of claim 107, wherein the T cell bispecific antibody comprises
(i) a first antigen binding moiety that binds to CD3 and comprises
(a) a heavy chain variable region comprising a heavy chain CDR (HCDR) 1 of SEQ ID NO: 61, a HCDR2 of SEQ ID NO: 29, and a HCDR3 of SEQ ID NO: 62, or a heavy chain variable region comprising a HCDR1 of SEQ ID NO: 64, a HCDR2 of SEQ ID NO: 29 and a HCDR3 of SEQ ID NO: 65, and
(b) a light chain variable region comprising a light chain CDR (LCDR) 1 of SEQ ID NO: 31, a LCDR2 of SEQ ID NO: 32 and a LCDR3 of SEQ ID NO: 33, and
(ii) a second antigen binding moiety that binds to CD19 and comprises
(a) a heavy chain variable region comprising a heavy chain CDR (HCDR) 1 of SEQ ID NO: 67, a HCDR2 of SEQ ID NO: 68, and a HCDR3 of SEQ ID NO: 69, and
(b) a light chain variable region comprising a light chain CDR (LCDR) 1 of SEQ ID NO: 70, a LCDR2 of SEQ ID NO: 71 and a LCDR3 of SEQ ID NO: 72.

109. The method of claim 108, wherein the T cell bispecific antibody further comprises (i) a third antigen binding moiety that binds to CD19, (ii) an Fc domain composed of a first and a second subunit, or (iii) both (i) and (ii).

110. The method of claim 109, wherein the T cell bispecific antibody comprises
(i) a first antigen binding moiety that binds to CD3, comprising
(a) a heavy chain variable region comprising a heavy chain CDR (HCDR) 1 of SEQ ID NO: 61, a HCDR2 of SEQ ID NO: 29, and a HCDR3 of SEQ ID NO: 62, or a heavy chain variable region comprising a HCDR1 of SEQ ID NO: 64, a HCDR2 of SEQ ID NO: 29 and a HCDR3 of SEQ ID NO: 65, and
(b) a light chain variable region comprising a light chain CDR (LCDR) 1 of SEQ ID NO: 31, a LCDR2 of SEQ ID NO: 32 and a LCDR3 of SEQ ID NO: 33,
wherein the first antigen binding moiety is a crossover Fab molecule wherein either the variable or the constant regions of the Fab light chain and the Fab heavy chain are exchanged;
(ii) a second and a third antigen binding moiety that bind to CD19, comprising
(a) a heavy chain variable region comprising a heavy chain CDR (HCDR) 1 of SEQ ID NO: 67, a HCDR2 of SEQ ID NO: 68, and a HCDR3 of SEQ ID NO: 69; and
(b) a light chain variable region comprising a light chain CDR (LCDR) 1 of SEQ ID NO: 70, a LCDR2 of SEQ ID NO: 71 and a LCDR3 of SEQ ID NO: 72,
wherein the second and third antigen binding moiety are each a Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

111. The method of claim 110, wherein the (a) first antigen binding moiety of the T cell bispecific antibody comprises a heavy chain variable region sequence that is at least about 95%, 96%, 97%, 98%, 99% or 100% identical to the amino acid sequence of SEQ ID NO: 63 or a heavy chain variable region sequence that is at least about 95%, 96%, 97%, 98%, 99% or 100% identical to the amino acid sequence of SEQ ID NO: 66, and a light chain variable region sequence that is at least about 95%, 96%, 97%, 98%, 99% or 100% identical to the amino acid sequence of SEQ ID NO: 35; or the (b) second or (c) third antigen binding moieties of the T cell bispecific antibody comprise a heavy chain variable region sequence that is at least about 95%, 96%, 97%, 98%, 99% or 100% identical to the amino acid sequence of SEQ ID NO: 73, and a light chain variable region sequence that is at least about 95%, 96%, 97%, 98%, 99% or 100% identical to the amino acid sequence of SEQ ID NO: 74, or any combination of (a), (b), and (c).

112. The method of claim 110, wherein the first antigen binding moiety of the T cell bispecific antibody is a crossover Fab molecule wherein the variable regions of the Fab light chain and the Fab heavy chain are exchanged, and wherein the second and third antigen binding moiety of the T cell bispecific antibody is a conventional Fab molecule wherein in the constant domain CL the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat) and in the constant domain CH1 the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

113. The method of claim 110, wherein the Fc domain of the T cell bispecific antibody comprises (i) a modification promoting the association of the first and the second subunit of the Fc domain, (ii) one or more amino acid substitution that reduces binding to an Fc receptor or effector function, or (iii) both (i) and (ii).

114. The method of claim 6, wherein the individual is being treated for a disease with the T cell bispecific antibody.

115. The method of claim 114, wherein the disease is cancer.

116. The method of claim 115, wherein the cancer expresses the target cell antigen of the T cell bispecific antibody.

117. The method of claim 116, wherein the target cell antigen is carcinoembryonic antigen (CEA).

118. The method of claim 117, wherein the cancer is colorectal cancer, lung cancer, pancreatic cancer, breast cancer, or gastric cancer.

119. The method of claim 116, wherein the target cell antigen is Wilms tumor protein (WT1).

120. the method of claim 119, wherein the cancer is a hematological cancer.

121. The method of claim 120, wherein the hematological cancer is leukemia.

122. The method of claim 121, wherein the leukemia is acute lymphocytic leukemia (ALL) or acute myeloid leukemia (AML).

123. The method of claim 116, wherein the target cell antigen is CD20.

124. The method of claim 123, wherein the cancer is a B-cell cancer

125. The method of claim 124, wherein the B-cell cancer is a Non-Hodgkin lymphoma (NHL), acute lymphocytic leukemia (ALL), chronic lymphocytic leukemia (CLL), diffuse large B-cell lymphoma (DLBCL), follicular lymphoma (FL), mantle-cell lymphoma (MCL), or marginal zone lymphoma (MZL).

126. The method of claim 116, wherein the target cell antigen is CD19.

127. The method of claim 126, wherein the cancer is a B-cell cancer.

128. The method of claim 127, wherein the B-cell cancer is a Non-Hodgkin lymphoma (NHL), acute lymphocytic leukemia (ALL), or chronic lymphocytic leukemia (CLL).

129. The method of claim 114, wherein the disease is an autoimmune disease.

130. The method of claim 129, wherein the autoimmune disease is lupus.

131. The method of claim 130, wherein the lupus is systemic lupus erythematosus (SLE) or lupus nephritis (LN).
